(19) United States
(12) Patent Application Publication
(10) Pub. No.: US 2020/0262864 A1
(43) Pub. Date: Aug. 20, 2020

(54) DEUTERATED FORMS OF AMINOSTEROLS AND METHODS OF USING THE SAME

(71) Applicant: Enterin, Inc., Philadelphia, PA (US)

(72) Inventors: Denise Barbut, Philadelphia, PA (US); Michael Zasloff, Philadelphia, PA (US); William A. Kinney, Newtown, PA (US); Stephen Jones, King of Prussia, PA (US); Scott Ashford, New York, NY (US); Anton Bieliauskas, New York, NY (US); Edward Hessler, Vicksburg, MI (US); Tom Fevig, Vicksburg, MI (US); Harold Karnes, Vicksburg, MI (US)

(73) Assignee: Enterin, Inc., Philadelphia (US)

(21) Appl. No.: 16/795,356

(22) Filed: Feb. 19, 2020

Related U.S. Application Data
(60) Provisional application No. 62/808,067, filed on Feb. 20, 2019.

(51) Int. Cl. C07J 41/00; C07J 21/00; C07C 269/06; C07C 271/20

(52) U.S. Cl. CPC C07J 41/0005; C07J 21/006; A61K 9/0053; C07C 271/20; C07B 2200/05; C07C 269/06

(57) ABSTRACT

Described are deuterated forms of aminosterols, or a pharmaceutically acceptable salt thereof, wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium.

### CROSS-REFERENCE TO RELATED APPLICATION

This application claims priority to U.S. Provisional Application No. 62/808,067, filed Feb. 20, 2019, the disclosure of which is specifically incorporated by reference in its entirety.

### BACKGROUND

Amino sterols are amino derivatives of a sterol. Exemplary aminosterols include squalamine and aminosterol 1436. The discovery of squalamine, a water-soluble compound, the structure of which is shown below, was reported by Michael Zasloff in 1993 (U.S. Pat. No. 5,192,756).

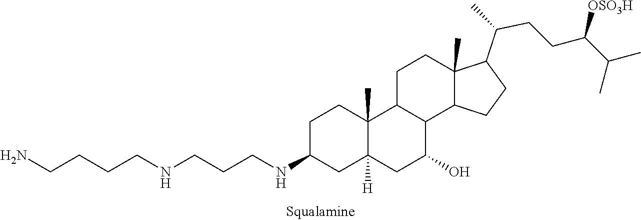

Chemically, squalamine presented a structure never before seen in nature, that being a bile acid coupled to a polyamine (spermidine); i.e., a steroid chemically linked to a polyamine. The chemical structure of squalamine, also known as 3 beta-N-1-(N-[3-(4-aminobutyl)]-1,3-diaminopropane)-7 alpha,24 zeta-dihydroxy-5 alpha-cholestane 24-sulfate, has been determined by fast atom bombardment mass spectroscopy and NMR. Squalamine is a cationic steroid characterized by a condensation of an anionic bile salt intermediate with spermidine.

Numerous studies later demonstrated that squalamine exhibits potent antibacterial activity in vitro (Salmi et al., 2008). Subsequently, squalamine was discovered to exhibit antiangiogenic activity in vitro and upon administration to animals (Sills et al., 1998; Yin et al., 2002). As a consequence, squalamine has been evaluated in disease states known to be associated with pathological neovascularization, such as cancer (Sills et al., 1998; Schiller, J. H. and G. Bittner, 1999; Bhargava et al., 2001; Williams et al., 2001; Hao et al., 2003; Herbst et al., 2003; Sokoloff et al., 2004), and vascular disorders of the eye, including macular degeneration (US 2007/10504A1), retinopathy of prematurity (Higgins et al., 2000; Higgins et al., 2004; US2007/10504A1), corneal neovascularization (Genaidy et al., 2002) and diabetic retinopathy (US 2007/10504A1).

The utility of squalamine as an anti-infective has been demonstrated in vitro against bacteria and fungi (Moore et al., 1993; Rao et al., 2000; Salmi et al., 2008). Squalamine is a cationic amphipathic substance exhibiting an affinity for membranes composed of anionic phospholipids (Selinsky et al., 1998; Selinsky et al., 2000). Squalamine is believed to exert antimicrobial action by interacting electrostatically with the membranes of target microorganisms, which generally display anionic phospholipids on the membrane surface exposed to the environment, subsequently disturbing their functional integrity, and causing death of the targeted microbe (Sills et al., 1998; Zasloff, M., 2002; Salmi et al., 2008).

Recent studies have highlighted the efficacy of systemically administered squalamine to prevent or treat viral infections in animals (Zasloff et al., 2011; U.S. Pat. No. 8,729,058).

The mechanism of action. It has been reported that squalamine exerts its effects at the cellular level by displacing proteins bound electrostatically to negatively charged membranes, causing pleiotropic changes in the functional state of the cell (Alexander et al., 2011; Yeung et al., 2008; Sumioka et al., 2009; Zasloff et al., 2011).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed Aug. 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed Dec. 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed Dec. 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed Mar. 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With ‘Wet’ Age-Related Macular Degeneration (AMD),” by Genaera Corporation (60 participants; study completed Feb. 2007);

(7) ClinicalTrials.gov Identifier NCT00089830 for “A Safety and Efficacy Study of MSI-1256F (Squalamine Lactate) To Treat ‘Wet’ Age-Related Macular Degeneration,” by Genaera Corporation (120 participants; study completed May 2007); and

(8) ClinicalTrials.gov Identifier NCT03047629 for Evaluation of Safety and Tolerability of ENT-01 for the Treatment of Parkinson's Disease Related Constipation (RASMET) (50 participants; study completed Jun. 14, 2018).

Squalamine is also marketed under the brand name Squalamax™ as a dietary supplement, though it has not been approved as a drug in this form and thus cannot make therapeutic claims. Squalamax™ is an unfractionated extract of shark liver, containing innumerable uncharacterized substances in addition to squalamine. Squalamine is present in Squalamax™ at less than 0.01% of the total weight of the extract. “Cyber Warning Letter”, Center for Drug Evaluation and Research (2002-May-6), http://www.fda.gov/CDER/warn/cyber/2002/CFSANnuGen.htm; Retrieved 2009-Mar.-31. Moreover, the dietary supplement form of squalamine is not pharmaceutical grade squalamine, as pharmaceutical grade squalamine requires significantly greater manufacturing efforts.

By 2006, over 300 patients had received squalamine in doses ranging from 6-700 mg/m2/day by iv administration, in three Phase I and nine Phase II studies. (Hao et al., 2003; Herbst et al., 2003; Bhargava et al., 2001; and Connolly et al., 2006). The studies showed that the compound exhibited an acceptable safety profile and evidence of efficacy in these early trials.

Squalamine derivatives. Analyses of larger quantities of dogfish liver extracts revealed squalamine to be the most abundant member of a larger aminosterol family comprising at least 12 related compounds (Rao et al., 2000). A known squalamine derivative is Aminosterol 1436, also known as MSI-1436 and trodusquemine. Although structurally similar to squalamine (it carries a spermine rather than a spermidine) and also quite potent as an anti-infective, Aminosterol 1436 exhibits a profoundly different pharmacology in vertebrates, causing weight loss and adipose tissue mobilization (Zasloff et al., 2001).

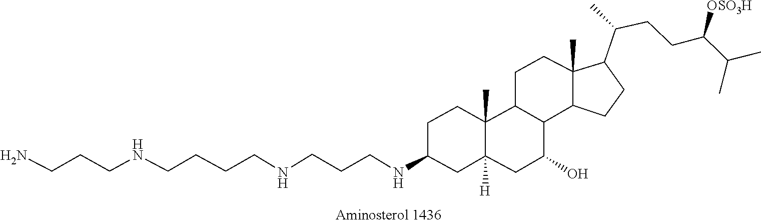

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

Given the potential clinical significance of aminosterols such as squalamine Aminosterol 1436, there is a need in the art for new forms of the drugs. The present invention satisfies this need.

### SUMMARY

In one aspect, provided herein are compounds having the structure of

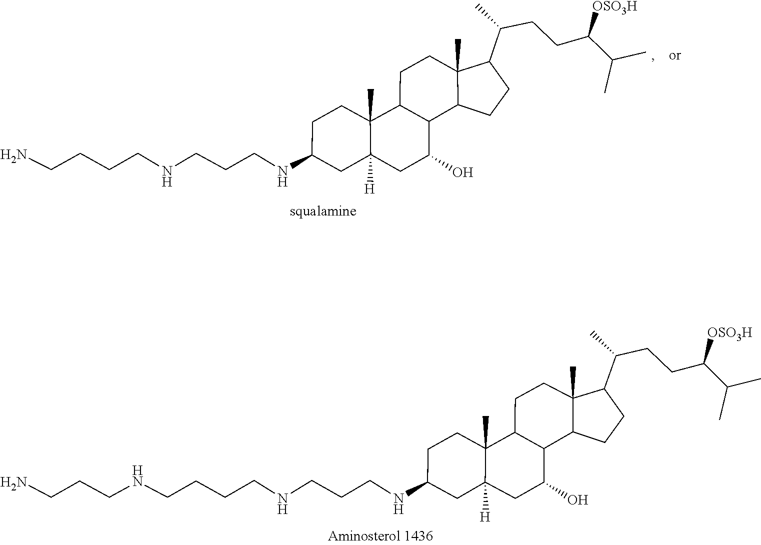

or a pharmaceutically acceptable salt thereof, wherein at least one hydrogen is replaced with deuterium.

In one aspect of the invention, encompassed are squalamine and/or aminosterol 1436 compounds wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium. In other embodiments (a) one or more hydrogen atoms at one or more positions selected from C2, C3, C4, C5, C6, C7, C8, C22, C23, C24, C25, C26, and C27, are replaced with deuterium; or (b) one or more hydrogen atoms at one or more positions selected from C3, C4, C5, C6, C7, C22, C23, C24, C25, C26, and C27, are replaced with deuterium; or (c) one or more hydrogen atoms at one or more positions selected from C4, C5, C7, C22, C23, C24, C25, C26, and C27, are replaced with deuterium; or (d) all hydrogen atoms at positions C25, C26, and C27 are replaced with deuterium; or (e) all hydrogen atoms at positions C26 and C27 are replaced with deuterium; or (f) one or more hydrogen atoms at one or more positions selected from C1, C2, C3, C4, C5, C6, C7, C8, C11, C12, C16, C17, C18, C19, C20, and C21 are replaced with deuterium.

In another aspect of the invention, for the deuterated aminosterol compounds described herein, any atom not designated as deuterium is present at its natural isotopic abundance. In another embodiment, the deuterium incorporation at each designated deuterium atom is at least about 90%, at least about 95%, or at least about 97%.

In yet another embodiment, the deuterated aminosterol compounds of the invention have an isotopic enrichment factor selected from the group consisting of at least about 3500, at least about 4000, at least about 4500, at least about 5000, at least about 5500, at least about 6000, at least about 6333.3, at least about 6466.7, at least about 6600, and at least about 6633.3. In one embodiment, the compounds of the invention have an isotopic enrichment factor of at least 3500 for one deuterium at a single position of the compound.

In one embodiment, the deuterated aminosterol compounds of the invention are a pharmaceutically acceptable salt, such as a phosphate salt.

In one embodiment, the deuterated aminosterol compounds of the invention have a longer half-life as compared to an undeuterated form of the same aminosterol. For example, the half-life can be increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect, provided herein are deuterated aminosterol compounds having a structure selected from the group consisting of:

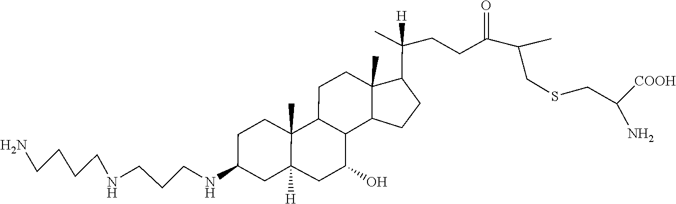 [SMILES: CC(CSCC(N)C(=O)O)C(=O)CC[C@@H](C)C1CCC2C3C(CC[C@@]21C)[C@@]1(C)CC[C@H](NCCCNCCCCN)C[C@@H]1C[C@H]3O]

Compound 1, or a pharmaceutically acceptable salt thereof;

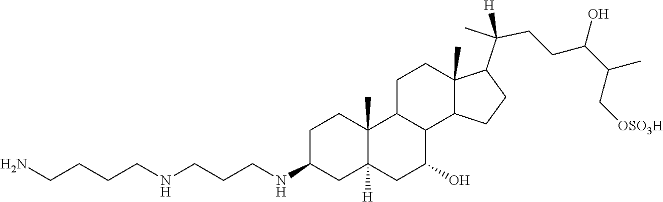

Compound 2, or a pharmaceutically acceptable salt thereof;

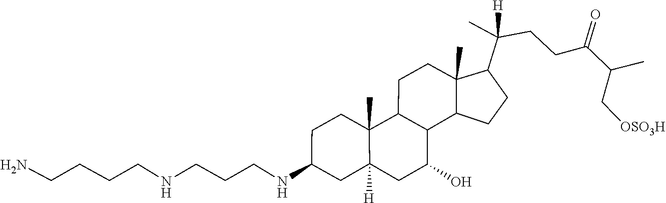

Compound 3, or a pharmaceutically acceptable salt thereof;

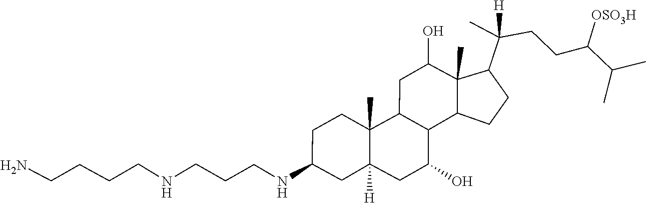

Compound 4, or a pharmaceutically acceptable salt thereof;

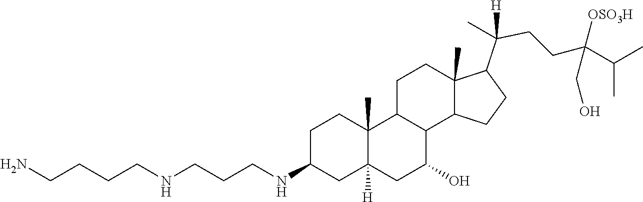

Compound 5, or a pharmaceutically acceptable salt thereof; and

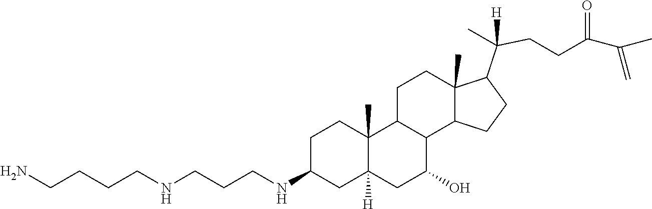 [SMILES: C=C(C)C(=O)CC[C@@H](C)C1CCC2C3C(CC[C@@]21C)[C@@]1(C)CC[C@H](NCCCNCCCCN)C[C@@H]1C[C@H]3O]

Compound 6, or a pharmaceutically acceptable salt thereof,

wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium.

In another aspect, provided herein are deuterated aminosterol compounds having a structure selected from the group consisting of:

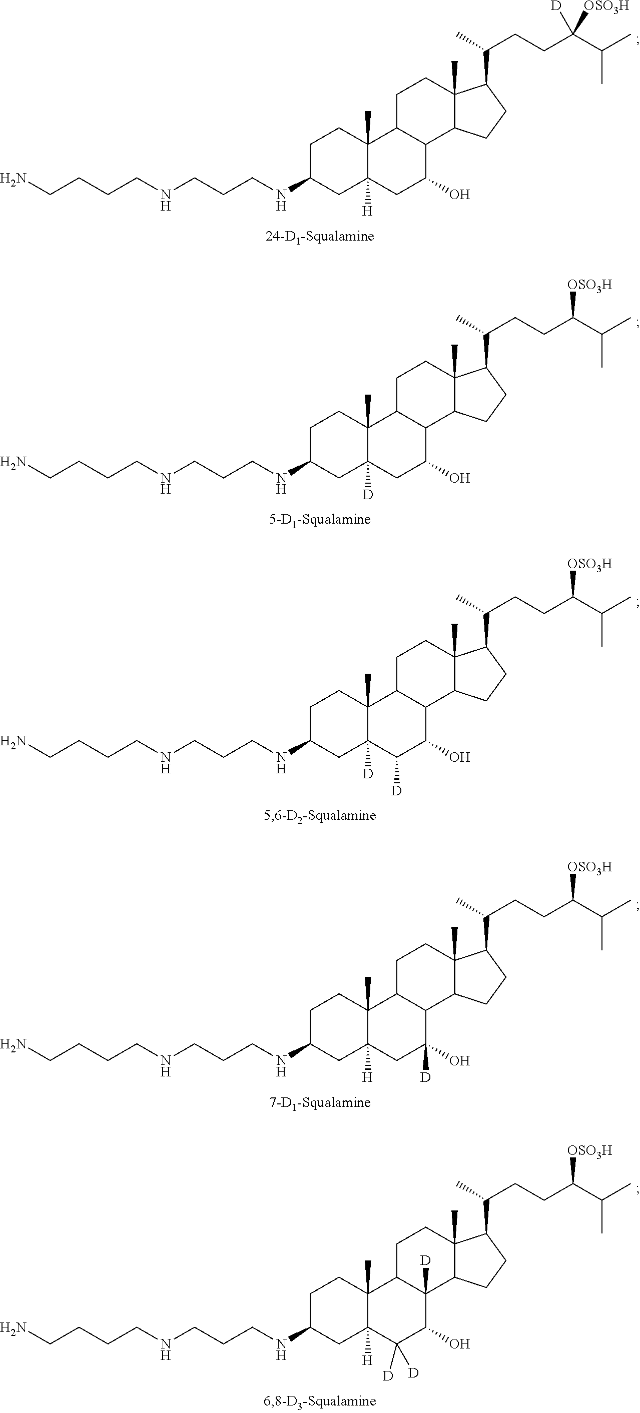

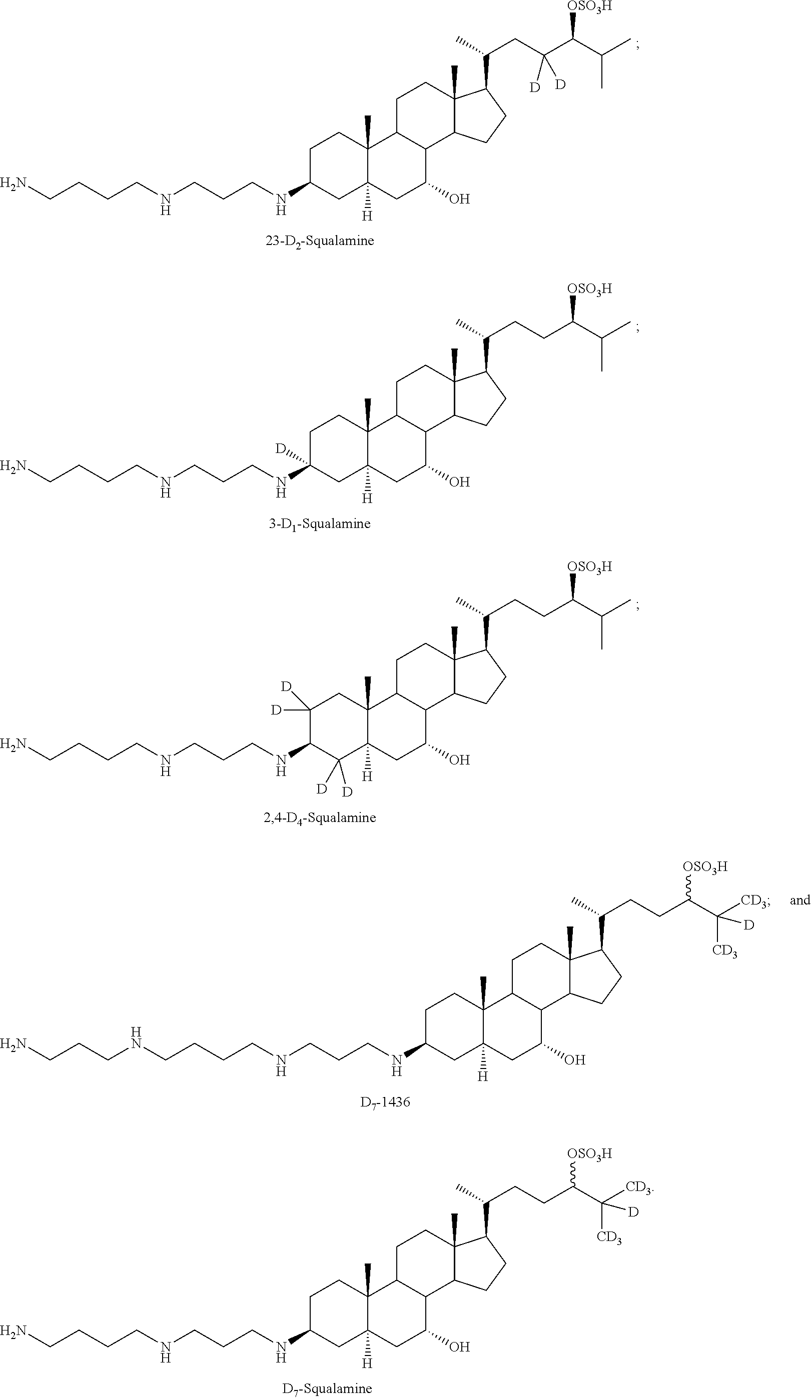

In another aspect, provided herein are pharmaceutical compositions comprising a compound disclosed herein and at least one pharmaceutically acceptable excipient or carrier. For example, the pharmaceutical composition can comprise one or more of an aqueous carrier, a buffer, a sugar and/or a polyol compound.

In another aspect, provided herein are methods for treating a subject in need having a condition susceptible to treatment with an aminosterol such as squalamine and/or aminosterol 1436, comprising administering to the subject a therapeutically effective amount of a deuterated compound disclosed herein. In the methods of the invention, the composition can be administered via any pharmaceutically acceptable means, such as for example orally, intranasally, or a combination thereof.

In one embodiment in the methods of the invention, the composition is administered orally and the dose of the compound or a salt or derivative thereof for the subject is from about 25 mg up to about 500 mg/day. In another embodiment, the composition is administered intranasally and the dose of the compound or a salt or derivative thereof for the subject is from about 0.001 mg up to about 6 mg/day.

In another embodiment in the methods of the invention, the method results in improvement or resolution of the condition, disease, or symptom as measured using a clinically recognized scale or tool. For example, the improvement in the condition, disease, or symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In one aspect in the methods of the invention, the deuterated aminosterol compound has an improved safety profile, as measured by a decrease in incidence of one or more adverse events evaluated using a clinically recognized scale or tool, as compared to the same aminosterol compound which has not been deuterated. For example, the safety profile can be improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect in the methods of the invention, the deuterated aminosterol compound has an improved efficacy as compared to the same aminosterol compound which has not been deuterated. For example, the improved efficacy can be measured by improvement of one or more disease symptoms evaluated using a clinically recognized scale or tool. For example, the efficacy improvement can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect in the methods of the invention, the deuterated aminosterol compound has an improved tolerability, measured using a clinically recognized scale or tool, as compared to the same compound which has not been deuterated. For example, the tolerability can be improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect in the methods of the invention, the deuterated aminosterol compound has a reduced required dosage amount and/or dosing frequency, as compared to the same aminosterol compound which has not been deuterated, to obtain the same or improved desired therapeutic effect as measured using a clinically recognized scale or tool. For example, the dosage can be about 10%, about 15%, about 20%, about 25%, about 30%, about 35%, about 40%, about 45%, or about 50% less than that of the same but undeuterated aminosterol.

As noted above, the methods of the invention can be used to treat any condition susceptible to treatment with an aminosterol such as squalamine and/or aminosterol 1436. Examples of conditions, diseases, or symptoms to be treated include, for example, (1) a gastrointestinal disorder selected from the group consisting of constipation, inflammatory bowel disease, irritable bowel syndrome, chronic idiopathic constipation and opioid induced constipation; (2) neurodegeneration or a neurological or neurodegenerative disorder; (3) a sleep disorder or sleep disturbance; (4) hallucinations; (5) depression; (6) schizophrenia; (7) an inflammatory disease or condition caused by excessive expression or concentration of alpha synuclein in the subject; (8) an infection selected from the group consisting of viral infections, antimicrobial infections, Gram-negative and Gram-positive bacterial infections, Mycobacteria infections, fungal infections, and protozoan infections; (9) a disease state known to be associated with pathological neovascularization, selected from the group consisting of cancer, vascular disorders of the eye, macular degeneration, age-related macular degeneration, retinopathy of prematurity, corneal neovascularization, diabetic retinopathy, fibrodysplasia ossificans progressiva, and disorders of neovascularization; and/or (10) obesity.

In one aspect the condition, disease, or symptom is constipation, and the therapeutically effective amount of the composition is defined as the amount that results in a complete spontaneous bowel movement (CSBM) within 24 hours of dosing on at least 2 of 3 days at a given dose.

In one aspect the condition, disease, or symptom is neurodegeneration or a neurological or neurodegenerative disorder, and (a) the neurodegeneration is age-related; (b) the neurodegeneration is correlated with age-related dementia; (c) the neurodegeneration is correlated with a neurodisease; and/or (d) the neurodegeneration is correlated with one or more conditions or diseases selected from the group consisting of Alzheimer's disease, Parkinson's disease, Lewy Body dementia or disease, fronto temperal dementia, supranuclear palsy, multi-system atrophy (MSA), Parkinsonism, amyotrophic lateral sclerosis (ALS), Huntington's chorea and/or Huntington's disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian Parkinsonism, spinocerebellar ataxia, hallucinations, depression, autism, vascular dementia, neuropathy of diabetes, peripheral sensory neuropathy, cerebral palsy, epilepsy, diabetic neuropathy, traumatic head and/or spine injury, stroke, or any combination thereof.

In another aspect the method results in slowing, halting, or reversing progression or onset of the neurodegeneration over a defined time period following administration of the composition, as measured by a medically-recognized technique; and/or the neurodegeneration is positively impacted by administration of the composition, as measured by a medically-recognized technique. For example, the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In another aspect, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

Also encompassed are methods of treating subjects at risk of developing, or suffering from, a sleep disorder or sleep disturbance. In one aspect, administration of the composition decreases the occurrence of at least one symptom of the sleep disorder or disturbance. In another aspect, the method can result in a positive change in the sleeping pattern of the subject. For example, the positive change can be defined as: (a) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (b) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In another aspect, as a result of the method the subject obtains the total number of hours of sleep recommended by a medical authority for the age group of the subject.

Examples of sleep disorders or sleep disturbances include, for example, circadian rhythm disruption or dysfunction, delays in sleep onset, fragmentation of sleep, reduced REM sleep, REM disturbed sleep, reduced total sleep time, REM-behavior disorder, sleep breathing disorder including snoring and sleep apnea, hallucinations, micro-sleep episodes, narcolepsy, sleep problems or sleep disturbances, day-time sleepiness, or any combination thereof. In one aspect, the REM-behavior disorder comprises vivid dreams, nightmares, and acting out the dreams by speaking or screaming, or fidgeting or thrashing of arms or legs during sleep. In another aspect, the sleep disorder comprises a loss of diurnal rhythm (Circadian rhythm). The loss of diurnal rhythm can be caused by, for example, dysfunction of the suprachiasmatic nucleus, dysfunction of the enteric nervous system, dysfunction of the olfactory nervous system, dysfunction of circadian rhythm caused by visual loss, dysfunction of circadian rhythm caused by jet lag and/or dysfunction of circadian rhythm caused by night-shift work. In one embodiment, administration of the composition reverses the dysfunction of the: (a) suprachiasmatic nucleus, restores the diurnal rhythm, and treats the sleep disorder; and/or (b) enteric nervous system, restores the diurnal rhythm, and treats the sleep disorder; (c) olfactory system, restores the diurnal rhythm, and treats the sleep disorder; (d) circadian rhythm caused by visual loss; (e) circadian rhythm caused by jet lag; and/or (f) circadian rhythm caused by night-shift work.

In another aspect, the sleep disorder is associated with a neurodegenerative disorder. In this embodiment, treating the sleep disorder can prevent or delay the onset or progression of the neurodegenerative disorder. The neurodegenerative disorder can be, for example, Alzheimer's disease, Parkinson's disease, Lewy Body dementia or disease, fronto temperal dementia, supranuclear palsy, multi-system atrophy (MSA), Parkinsonism, amyotrophic lateral sclerosis (ALS), Huntington's chorea and/or Huntington's disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian Parkinsonism, spinocerebellar ataxia, hallucinations, depression, autism, vascular dementia, neuropathy of diabetes, peripheral sensory neuropathy, cerebral palsy, epilepsy, diabetic neuropathy, traumatic head and/or spine injury, stroke, or any combination thereof.

In another aspect, the subject to be treated suffers from, is or at risk of developing, hallucinations. Administration of the composition of the invention can result in a decreased number or severity of hallucinations of the subject; and/or result in the subject being hallucination-free. For example, the decrease in number or severity in hallucinations can be defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

The hallucination can comprise a visual, auditory, tactile, gustatory or olfactory hallucination. The hallucination can be the result of, for example, a neurodegenerative disorder, a psychiatric disorder, a neurological disorder, a brain tumor, a sensory loss, and/or dysfunction of the enteric nervous system. The sensory loss can be, for example, visual, auditory, gustatory, tactile, or olfactory. The neurological or neurodegenerative disorder can be, for example, Alzheimer's disease, Parkinson's disease, Lewy Body dementia or disease, fronto temperal dementia, supranuclear palsy, multi-system atrophy (MSA), Parkinsonism, amyotrophic lateral sclerosis (ALS), Huntington's chorea and/or Huntington's disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian Parkinsonism, spinocerebellar ataxia, hallucinations, depression, autism, vascular dementia, neuropathy of diabetes, peripheral sensory neuropathy, cerebral palsy, epilepsy, diabetic neuropathy, traumatic head and/or spine injury, stroke, or any combination thereof. The neurodegenerative or neurological disorder can be the result of, for example, a sleep disorder; a focal brain lesion; a focal brain lesion which is occipital lobe lesions or temporal lobe lesions; a temporal lobe lesion selected from the group consisting of lesions of the uncinate gyrus, cerebral peduncles, and substantia nigra; a diffuse involvement of the cerebral cortex, a diffuse involvement of the cerebral cortex caused by a viral infectious disease; a diffuse involvement of the cerebral cortex caused by a viral infectious disease, wherein the viral infectious disease is selected from the group consisting of acute metabolic encephalopathies, encephalitis, and meningitis; a diffuse involvement of the cerebral cortex caused by a cerebral vasculitis condition; a diffuse involvement of the cerebral cortex caused by a cerebral vasculitis condition, wherein the cerebral vasculitis condition is caused by an autoimmune disorder, a bacterial or viral infection, or a systemic vasculitis; and/or a diffuse involvement of the cerebral cortex caused by a cerebral vasculitis condition, wherein the cerebral vasculitis condition is caused by an autoimmune disorder which is Systemic Lupus Erythematosus (SLE). The psychiatric disorder can be, for example, Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, or Schizophrenia.

Administration of the composition of the invention can (a) reverse dysfunction caused by the neurodegenerative or neurological disorder and treats and/or prevents the hallucination; (b) reverse dysfunction caused by the psychiatric disorder and treats and/or prevents the hallucination; (c) reverse dysfunction caused by the sensory loss and treats and/or prevents the hallucination; and/or (d) reverse dysfunction of the enteric nervous system and treats and/or prevents the hallucination.

In another aspect, the subject to be treated suffers from, is or at risk of developing, depression. The method can result in improvement in a subject's depression, as measured by one or more clinically-recognized depression rating scale. For example, the improvement can be in one or more depression characteristics selected from the group consisting of mood, behavior, bodily functions such as eating, sleeping, energy, and sexual activity, and/or episodes of sadness or apathy; and/or the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In another aspect, the subject to be treated suffers from, is or at risk of developing, autism. In one aspect, the method results in improvement in one or more of the subject's autism characteristics or behaviors, as measured by a clinically-recognized rating scale; and/or in one or more autism characteristics or behaviors selected from the group consisting of social skills, repetitive behaviors, speech, nonverbal communication, sensory sensitivity, behavior, social interaction, and communication skills, as measured using a clinically-recognized scale. For example, the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%.

In another aspect, the subject to be treated suffers from, is or at risk of developing, schizophrenia. In one aspect, the method results in improvement in one or more schizophrenia characteristics or behaviors, as measured using a clinically recognized rating scale. For example, the schizophrenia characteristics or behaviors can be selected from the group consisting of unclear or confusing thinking, reduced social engagement, reduced emotional expression, abnormal social behavior, failure to understand reality, lack of motivation, and hearing voices that others do not hear, as measured using a clinically-recognized scale. In another aspect, the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%.

In another aspect, the subject to be treated suffers from, is or at risk of developing, an inflammatory disease or condition caused by excessive expression or concentration of alpha synuclein in the subject. In another aspect, the method is applied to a patient population susceptible to excessive expression of alpha-synuclein, resulting in an excessive or high concentration of alpha-synuclein. In one aspect, the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment. For example, the method can result in a decrease in concentration of alpha synuclein in the subject. The decrease in alpha-synuclein concentration can be measured qualitatively, quantitatively, or semi-quantitatively by one or more methods selected from the group consisting of: (a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control; (b) measuring the intensity of inflammation over time; (c) measuring the amount of inflammatory markers over time; (d) measuring the amount of inflammatory markers in blood, plasma, or tissue over time, either qualitatively or quantitatively; (e) measuring the amount of one or more inflammatory marker cytokines in blood, plasma, or tissue over time, either qualitatively or quantitatively; (f) measuring the amount of one or more plasma markers of inflammation such as TNF, IL-8, or CRP in blood, plasma, or tissue over time, either qualitatively or quantitatively; and (g) measuring the amount of inflammatory cells in blood, plasma, or tissue over time, either qualitatively or quantitatively. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one aspect, the subject to be treated has an infection selected from the group consisting of viral infections, antimicrobial infections, Gram-negative and Gram-positive bacterial infections, Mycobacteria infections, fungal infections, and protozoan infections. In another aspect, the subject to be treated has a disease state known to be associated with pathological neovascularization, selected from the group consisting of cancer, vascular disorders of the eye, macular degeneration, age-related macular degeneration, retinopathy of prematurity, corneal neovascularization, diabetic retinopathy, fibrodysplasia ossificans progressiva, and disorders of neovascularization. In another aspect, the subject to be treated suffers from obesity, a need for weight loss or weight management, and/or dose-dependent weight loss; and/or diseases, including viral infections, where sodium-hydrogen exchanger (“NHE-3”) plays a critical role. In some embodiments, the condition to be treated is selected from the group consisting of AIDS, viral meningitis, Dengue, EBV, hepatitis, a chronic disease suspected to be of viral origin, multiple sclerosis, Type I diabetes, Type II diabetes, atherosclerosis, cardiomyopathies, Kawaski disease, aplastic anemia, and any combination thereof.

In some embodiments, the composition is taken on an empty stomach, optionally within two hours of the subject waking. In some embodiments, no food is taken after about 60 to about 90 minutes of taking the composition.

In some embodiments, the pharmaceutical composition further comprises administering an additional active agent to achieve either an additive or synergistic effect. The additional active agent can be administered via any pharmaceutically acceptable method and includes, for example, administration concomitantly, as an admixture, separately and simultaneously or concurrently, or separately and sequentially. In one aspect, the additional active agent is a different aminosterol from the first aminosterol administered in the method of the invention. In another aspect, the invention encompasses a method comprising (a) orally administering a composition comprising a deuterated or nondeuterated aminosterol compound according to the invention and (b) intranasally administering a composition comprising a deuterated or nondeuterated aminosterol compound, wherein at least one of the aminosterol compounds of (a) or (b) is deuterated. In some embodiments, the methods of the invention comprise treating a human subject.

Another aspect of the invention encompasses methods of making deuterated aminosterol compounds. In a first method, encompassed is a process for preparing D₇-1436, the process comprising (a) reacting compound (A)

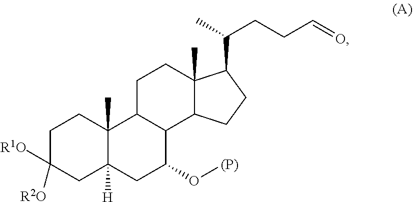

with an organometallic reagent to provide compound (B):

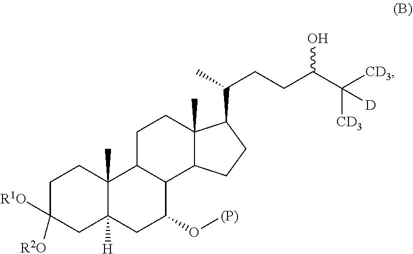

wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether; (b) reacting compound (B) with sulfur trioxide-pyridine complex to provide compound (C)

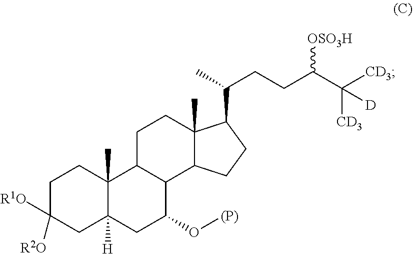

(c) reacting compound (C) under acidic conditions to provide compound (D)

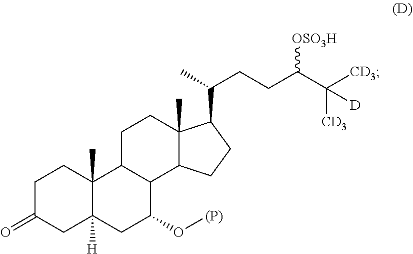

(d) reacting compound (D) under reductive amination conditions to provide compound (E)

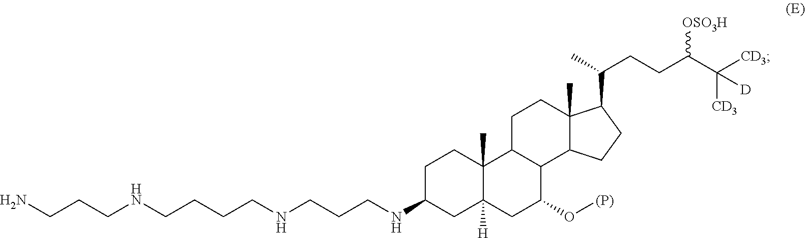

and
(e) reacting compound (E) under conditions to remove P to provide D₇-1436

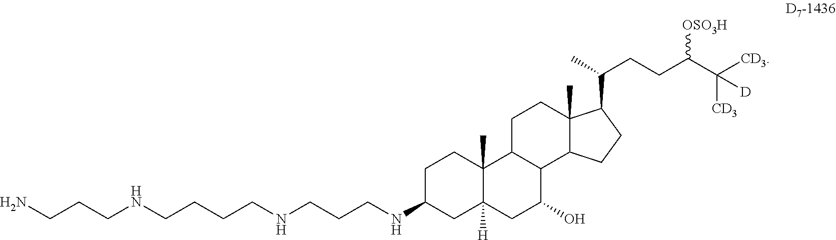

In a second method, encompassed is a process for preparing D₇-Squalamine, the process comprising (a) reacting compound (A)

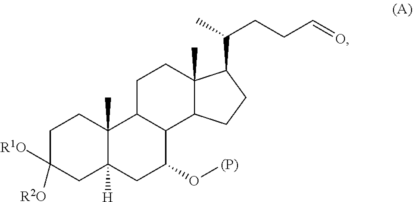

with an organometallic reagent to provide compound (B):

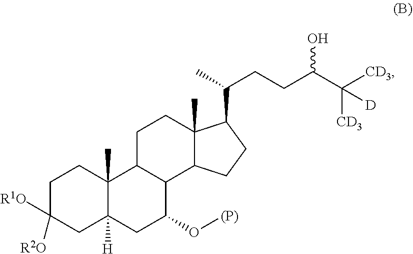

wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether; (b) reacting compound (B) with sulfur trioxide-pyridine complex to provide compound (C)

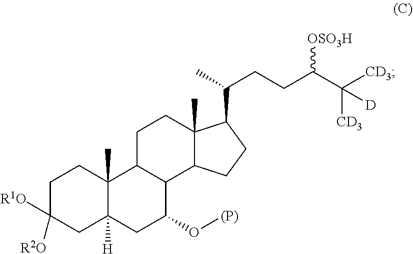

(c) reacting compound (C) under acidic conditions to provide compound (D)

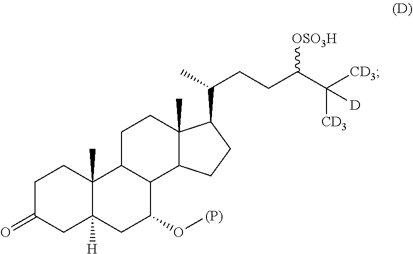

(d) reacting compound (D) under reductive amination conditions to provide compound (F)

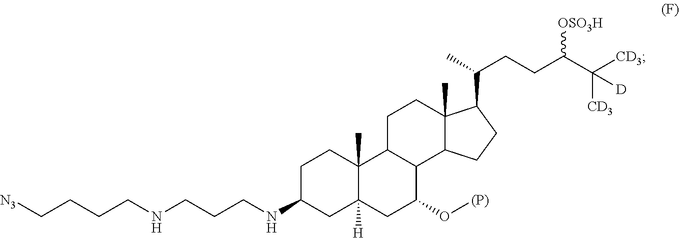

(e) reacting compound (F) under reducing conditions to provide compound (G)

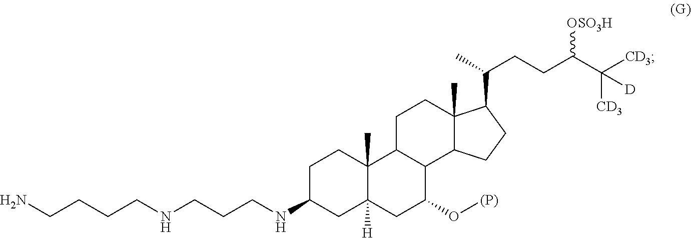

and
(f) reacting compound (G) under conditions to remove P to provide D₇-Squalamine

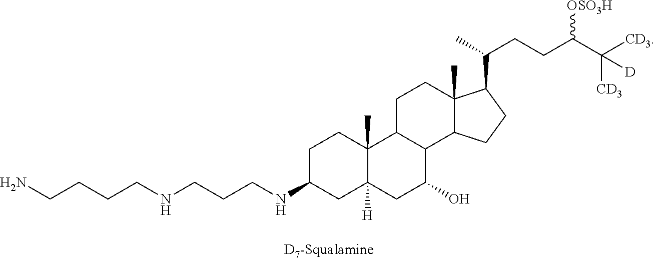

In a third method, encompassed is a process for preparing compound (O), the process comprising (a) reacting compound (H)

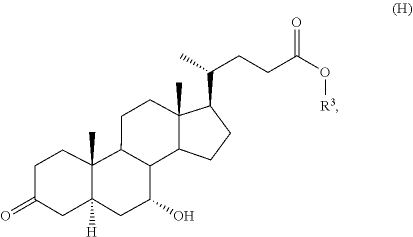

under acidic conditions to provide compound (J)

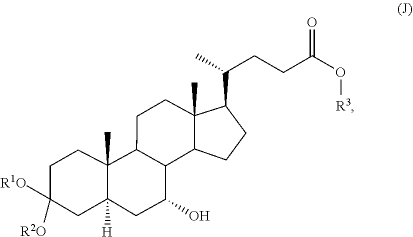

wherein each of R¹ and R² is independently C₁₋₆ alkyl, or R¹ and R², together with the oxygen atoms to which they are attached, form a heterocyclic ring; and R³ is substituted or unsubstituted C₁₋₆ alkyl; (b) reacting compound (J) with a reducing agent to provide compound (K)

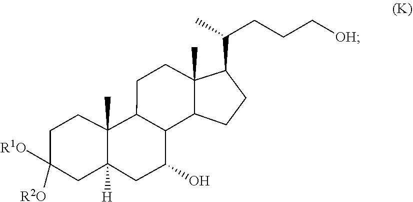

(c) reacting compound (K) under conditions to introduce a protecting group P′ to provide compound (L)

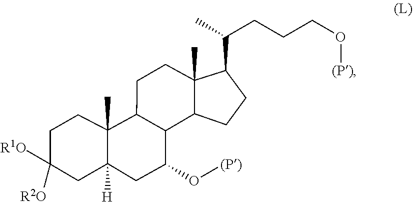

wherein P′ is substituted or unsubstituted C₁₋₆ alkyl ester, substituted or unsubstituted aryl ester, or substituted or unsubstituted heteroaryl ester; (d) reacting compound (L) under conditions to selectively remove one P′ to provide compound (M)

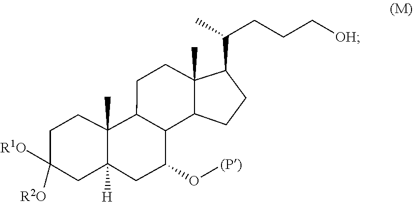

(e) reacting compound (M) under oxidation conditions to provide compound (N)

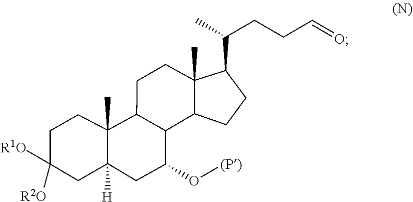

and
(f) reacting compound (N) with an organometallic reagent to provide compound (O)

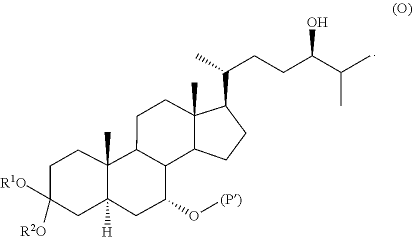

In a fourth method, encompassed is a process for preparing compound (O), the process comprising (a) reacting compound (P)

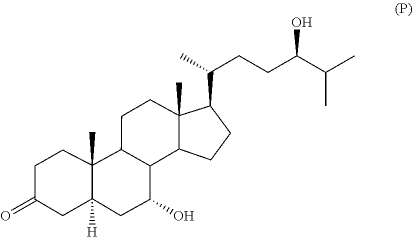 [SMILES: CC(C)[C@H](O)CC[C@@H](C)[C@H]1CCC2C3C(CC[C@@]21C)[C@@]1(C)CCC(=O)C[C@@H]1C[C@H]3O]

under acidic conditions to provide compound (Q):

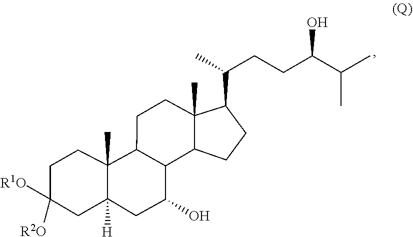

wherein each of R¹ and R² is independently C₁₋₆ alkyl, or R¹ and R², together with the oxygen atoms to which they are attached, form a heterocyclic ring; (b) reacting compound (Q) under conditions to introduce a protecting group P′ to provide compound (R)

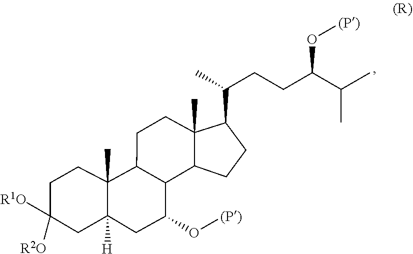

wherein P′ is substituted or unsubstituted C₁₋₆ alkyl ester, substituted or unsubstituted aryl ester, or substituted or unsubstituted heteroaryl ester; and (c) reacting compound (R) under conditions to selectively remove one P′ to provide compound (O)

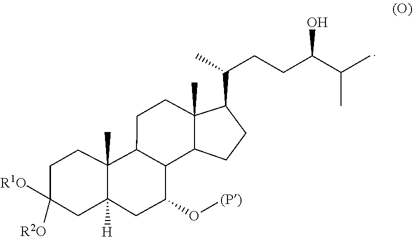

In a fifth method, encompassed is a process for preparing CBZ-spermidine:

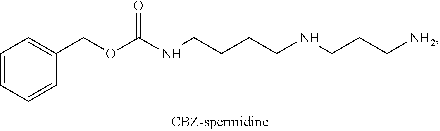 [SMILES: NCCCNCCCCNC(=O)OCc1ccccc1]

or a salt thereof, the process comprising (a) reacting 4-amino-1-butanol under conditions to introduce a Cbz group to provide compound (S)

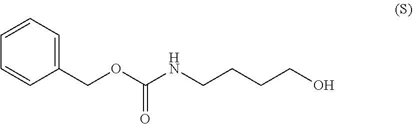 [SMILES: O=C(NCCCCO)OCc1ccccc1]

(b) sulfonylating compound (S) to provide compound (T)

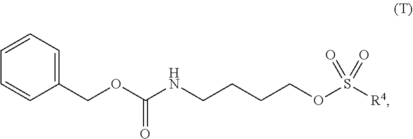

wherein R⁴ is substituted or unsubstituted C₁₋₆ alkyl, or substituted or unsubstituted aryl; and (c) reacting compound (T) with 1,3-diaminopropane to provide CBZ-spermidine, or the salt thereof.

Finally, the invention encompasses the following compounds and compositions comprising the same:

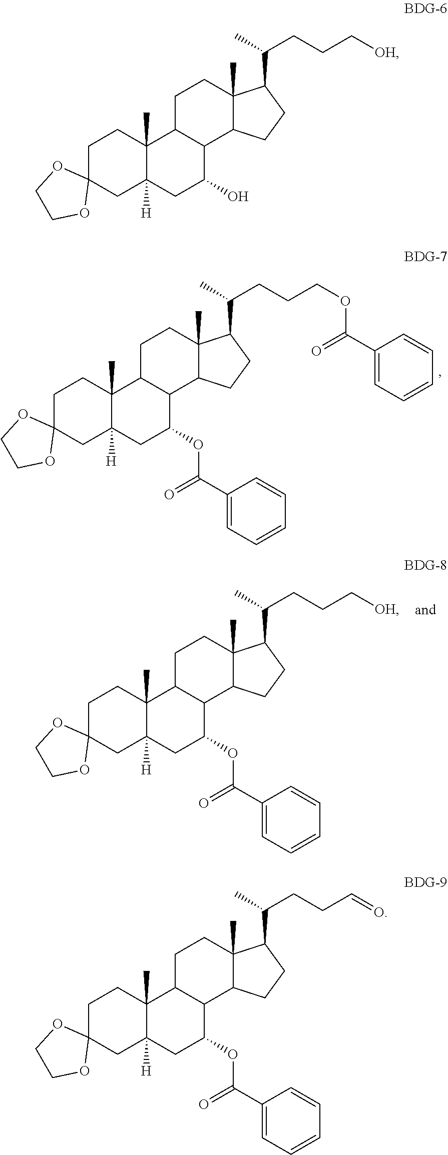

Both the foregoing summary of the invention and the following detailed description of the invention are exemplary and explanatory and are intended to provide further details of the invention as claimed. Other objects, advantages, and novel features will be readily apparent to those skilled in the art from the following detailed description of the invention.

### BRIEF DESCRIPTION OF THE DRAWINGS

FIGS. 1A-1B shows prokinetic activity of squalamine (ENT-01, a synthetic squalamine salt comprising squalamine as the active ion). As shown in panel A, in Stage 1 (single dose), cumulative prokinetic response rate was defined as the proportion of patients who had a complete spontaneous bowel movements (CSBM) within 24 hours of dosing. In Stage 2 (daily dosing), a prokinetic response was defined as the fraction of patients who had a CSBM within 24 hours of dosing on at least 2 out of 3 days at any given dose. As shown in panel B, the prokinetic dose of squalamine was significantly related to baseline constipation severity (p=0.00055). Patients with baseline CSBM <1 required a higher dose (mean, 192 mg) of squalamine than patients with CSBM ≥1 (mean, 120 mg).

FIG. 2 is a schematic (flowchart) showing patient disposition in Stage 2. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

FIG. 3 is a chart of total sleep time in relation to squalamine dose. Total sleep time was obtained from the sleep diary by subtracting awake time during the night from total time spent in bed. Total sleep time per night was logged for each patient at baseline, each dosing period and at washout, and the means were determined. The light grey bar represents the baseline value for each cohort at a given dose level and the dark grey bar represents the value for the same cohort at the stated dose of squalamine (ENT-01; Kenterin™). The number of patients represented at each value are: Baseline, 33; 75 mg, 21; 100 mg, 28; 125 mg, 18; 150 mg, 15; 175 mg, 12; 200 mg, 7; 225 mg, 3; 250 mg, 2; washout, 33. P values were as follows: 75 mg, p=0.4; 100 mg, p=0.1; 125 mg, p=0.3; 150 mg, p=0.07; 175 mg, p=0.03; 200 mg, p=0.3; 225 mg, p=0.5; 250 mg, p=0.3; wash-out, p=0.04 (paired t test).

FIG. 4 shows the effect of squalamine (ENT-01) on circadian rhythm. The figure depicts the mean waveform of temperature under three conditions per patient: baseline (Line #1), treatment with highest drug dose (Line #2), and washout (Line #3). Each mean waveform is double plotted for better visualization. Low temperatures indicate higher activation, while higher values are associated with drowsiness and sleepiness. The top black bar indicates a standard rest period from 23:00 to 07:00h.

FIGS. 5A-5F show the effect of squalamine (ENT-01) on circadian rhythm. The figures depict the results of circadian non-parametric analysis of wrist skin temperature rhythm throughout each condition (baseline, treatment with highest dose of squalamine (ENT-01) and washout). The following parameters were measured: Inter-daily variability (FIG. 5A), inter-daily stability (IS) (FIG. 5B), relative amplitude (RA) (FIG. 5C), circadian function index (FIG. 5D), M5V (FIG. 5E), which refers to the five consecutive hours with the highest temperature or high somnolence, and L10V (FIG. 5F), which indicates the mean of the ten consecutive hours with lowest temperature or high activation. The circadian function index (CFI) is an integrated score that ranges from 0 (absence of circadian rhythm) to 1 (robust circadian rhythm). Student's paired t-test, *p<0.05, **p<01, ***p<0.001. Values expressed as mean±SEM (n=12 in each condition).

FIG. 6 shows a single X-Ray of (24R)-Compound 34.

FIG. 7 shows weight of mice treated with D-1436 or MSI-1436. The y-axis is percent body weight, and the x-axis is time measured in days.

### DETAILED DESCRIPTION

The present technology relates to deuterated forms of aminosterols, or a pharmaceutically acceptable salt thereof, methods of preparing such forms, compositions comprising one or more of the deuterated forms of an aminosterol, or a pharmaceutically acceptable salt thereof, and methods of use thereof. A deuterated form of an aminosterol may have improved safety, better tolerability and/or enhanced efficacy. Deuterium is a naturally-occurring, stable, non-radioactive isotope of hydrogen.

Deuterated aminosterols, where one or more of the hydrogen atoms contained in the aminosterol molecule have been replaced by its heavier stable isotope deuterium, are anticipated to have a lower rate of metabolism and therefore a longer half-life and/or reduced or less frequent dosing to obtain a desired therapeutic effect. Deuterium-carbon bonds are generally about six to 10 times more stable than the corresponding hydrogen-carbon bond. These stronger bonds are more difficult to break, which can slow the rate of bond cleavage. This effect upon rate is called the kinetic isotope effect (KIE).

Deuteration of aminosterols can produce compounds having improved pharmacokinetic and/or toxicological properties, such as improved safety, efficacy and/or tolerability, as compared to unmodified aminosterols, due the stronger deuterium-carbon bond modifying the metabolism of the amino sterols.

In one embodiment, a deuterated aminosterol according to the invention has an improved safety profile, as measured by a decrease in incidence of one or more adverse events. For example, the increase in safety profile can be an increase of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment, a deuterated aminosterol according to the invention has improved efficacy. Efficacy can be measured, for example, by reduction of a disease symptom, improvement in disease or disorder characteristics, etc., all of which can be evaluated using a clinically recognized scale or tool. The improved efficacy can be an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In yet another embodiment, a deuterated aminosterol according to the invention has improved tolerability. Tolerability can be measured, for example, by reduction in one or more adverse events, or the ability to increase a dosage without triggering significant adverse events. The improved tolerability can be an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In yet another embodiment, a deuterated aminosterol according to the invention has a reduced required dosage amount, or dosing frequency, as compared to an unmodified aminosterol, to obtain the same or improved desired therapeutic effect. For example, a deuterated aminosterol composition according to the invention, can have a dosage about 5% less than the same but unmodified aminosterol to obtain the same therapeutic effect. In other embodiments, the dosage is about 10%, about 15%, about 20%, about 25%, about 30%, about 35%, about 40%, about 45%, or about 50% less than that of the same but unmodified aminosterol to obtain the same or improved therapeutic effect.

An “aminosterol” can be squalamine or a derivative thereof, Aminosterol 1436 or a derivative thereof, or a naturally occurring aminosterol isolated from Squalus acanthias or a derivative thereof, collectively referred to herein as “aminosterols.”

It will be recognized that some variation of natural isotopic abundance occurs in a synthesized compound depending upon the origin of chemical materials used in the synthesis. Thus, a preparation of aminosterols will inherently comprise small amounts of deuterated isotopologues. The concentration of naturally abundant stable hydrogen and carbon isotopes, notwithstanding this variation, is small and immaterial as compared to the degree of stable isotopic substitution of compounds of the present technology.

In a compound of the present technology, when a particular position is designated as having deuterium, it is understood that the abundance of deuterium at that position is substantially greater than the natural abundance of deuterium, which is 0.015%. Unless otherwise stated, when a position is designated specifically as “D” or “deuterium,” the position is understood to have deuterium at an abundance that is at least about 3340 times better than the natural abundance of deuterium, which is 0.015% (i.e., at least 50.1% incorporation of deuterium).

The term “isotopic enrichment factor” as used herein means the ratio between the isotopic abundance and the natural abundance of that isotope.

In some embodiments, a compound of this technology has an isotopic enrichment factor for each deuterium present at a site designated as a potential site of deuteration on the compound of at least about 3500 (52.5% deuterium incorporation), at least about 4000 (60% deuterium incorporation), at least about 4500 (67.5% deuterium incorporation), at least about 5000 (75% deuterium), at least about 5500 (82.5% deuterium incorporation), at least about 6000 (90% deuterium incorporation), at least about 6333.3 (95% deuterium incorporation), at least about 6466.7 (97% deuterium incorporation), at least about 6600 (99% deuterium incorporation), or at least about 6633.3 (99.5% deuterium incorporation).

The term “isotopologue” refers to a species that differs from a specific compound of this technology only in the isotopic composition thereof. Isotopologues can differ in the level of isotopic enrichment at one or more positions and/or in the positions(s) of isotopic enrichment.

The term “compound,” when referring to a compound of this technology, refers to a collection of molecules having an identical chemical structure, except that there may be isotopic variation among the constituent atoms of the molecules. Thus, it will be clear to those of skill in the art that a compound represented by a particular chemical structure comprising indicated deuterium atoms, will also comprise lesser amounts of isotopologues having hydrogen atoms at one or more of the designated deuterium positions in that structure. The relative amount of such isotopologues in a compound of this technology will depend upon a number of factors including the isotopic purity of deuterated reagents used to make the compound and the efficiency of incorporation of deuterium in the various synthesis steps used to prepare the compound.

In one embodiment, as set forth above the relative amount of such isotopologues in total will be less than about 49.9% of the compound. In other embodiments, the relative amount of such isotopologues in total will be less than about 47.5%, less than about 45%, less than about 40%, less than about 35%, less than about 32.5%, less than about 30%, less than about 25%, less than about 20%, less than about 17.5%, less than about 15% less than about 10%, less than about 5%, less than about 3%, less than about 1%, or less than about 0.5% of the compound.

### Example Data

Example 8 below describes an exemplary method of treating and/or preventing symptoms of Parkinson's disease (PD) in a clinical trial setting using an undeuterated aminosterol, squalamine phosphate (ENT-01). While the example describes data relating to undeuterated squalamine, the data described herein is predictive of success likely to be obtained with a deuterated aminosterol such as deuterated squalamine, as deuteration is not known to change the biological activity of a compound.

As described in Example 8, a study was conducted in patients with Parkinson's disease (PD). PD is a progressive neurodegenerative disorder caused by accumulation of the protein α-synuclein (αS) within the enteric nervous system (ENS), autonomic nerves and brain.

While the study described herein assessed patients with PD, many symptoms assessed and contemplated to be resolved by deuterated aminosterol treatment are not restored by the replacement of dopamine and are, thus, not unique to PD but rather common across a variety of disorders which involve impaired function of neural pathways, referred to herein as “brain-gut” disorders. Examples of such symptoms include, but are not limited to, constipation, disturbances in sleep architecture, cognitive impairment or dysfunction, hallucinations, REM behavior disorder (RBD), and depression. Other relevant symptoms are described herein. All of all of these symptoms result from impaired function of neural pathways not restored by replacement of dopamine.

In 2003, Braak proposed that PD begins with the formation of toxic αS aggregates within the ENS and manifests clinically as constipation in a majority of people years before the onset of motor symptoms. It was recently reported that αS is induced in the ENS in response to viral, bacterial and fungal infections and that excessive intraneuronal accumulation of αS promotes formation of toxic aggregates. As a result of the normal trafficking of αS aggregates from the ENS to the central nervous system (CNS) via afferent nerves such as the vagus, neurotoxic aggregates accumulate progressively within the brainstem and more rostral structures. Thus, inhibiting αS aggregation in the ENS may reduce the continuing PD disease process in both the ENS and CNS.

A strategy that targets neurotoxic aggregates of αS in the gastrointestinal tract represents a novel approach to the treatment of PD and other neurodiseases and conditions described herein that may restore the function of enteric nerve cells and prevent retrograde trafficking to the brain. Such actions may potentially slow progression of the disease in addition to restoring gastrointestinal function.

Accordingly but not to be bound by theory, the methods described herein are expected to apply to the treatment of any of the described symptoms as well as treatment and/or prevention of brain-gut disorders other than PD sharing such symptoms. Examples of such brain-gut disorders include but are not limited to (i) age-related neurodegeneration, (ii) age-related neurodegeneration correlated with age-related dementia, (iii) neurodiseases such as Alzheimer's disease (AD), Huntington's Disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, and autism.

Not to be bound by theory, it is believed that aminosterols target neurotoxic aggregates of αS in the gastrointestinal tract, and restore function of the enteric nerve cells. The now-functional enteric nerve cells prevent retrograde trafficking of proteins, such as alpha-synuclein, to the brain. In addition to restoring gastrointestinal function, this effect is believed to slow and possibly reverse disease progression.

Constipation serves as an early indicator of many neurodiseases such as PD to the extent that it is suspected to correlate with the formation of toxic αS aggregates within the enteric nervous system (ENS) (Braak et al. 2003). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008). This relationship between the ENS and CNS is sometimes described herein as “brain-gut” in relation to a class of disorders or the axis of aminosterol activity.

Not to be bound by theory, based on the data described herein, it is believed that deuterated aminosterols improve bowel function by acting locally on the gastrointestinal tract (as supported by the oral bioavailability <0.3%). An orally administered aminosterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in the study detailed in Example 8 and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 8 to evaluate the impact of an aminosterol on neurologic symptoms associated with a neurodisease such as PD. Following aminosterol treatment, the Unified Parkinson's Disease Rating Scale (UPDRS) score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 11).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose (e.g., fixed escalated aminosterol dose) of 175 mg for this particular patient. The patient remained free of hallucinations for 1 month following cessation of dosing. RBD and total sleep time also improved progressively in a dose-dependent manner.

Interestingly, most indices related to bowel function returned to baseline value by the end of the 2-week wash-out period while improvement in the CNS symptoms persisted. The rapid improvement in certain CNS symptoms is consistent with a mechanism whereby nerve impulses initiated from the ENS following aminosterol administration augment afferent neural signaling to the CNS. This may stimulate the clearance of tS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of aminosterol administration, the neurons of the CNS gradually re-accumulate an αS burden either locally or via trafficking from αS re-aggregation within the gut.

Disturbance of the circadian rhythm has been described in neurodiseases such as PD both clinically and in animal models and might play a role in the abnormal sleep architecture, dementia, mood and autonomic dysfunction associated with neurodiseases such as PD (Breen et al. 2014; Videnovic et al. 2017; Antonio-Rubio et al. 2015; Madrid-Navarro et al. 2018). Circadian rhythm was monitored through the use of a temperature sensor that continuously captured wrist skin temperature (Sarabia et al. 2008), an objective measure of the autonomic regulation of vascular perfusion (Videnovic et al. 2017). Circadian cycles of wrist skin temperature have been shown to correlate with sleep wake cycles, reflecting the impact of nocturnal heat dissipation from the skin on the decrease in core temperature and the onset of sleep (Sarabia et al. 2008; Ortiz-Tuleda et al. 2014). Oral administration of ENT-01 had a significant positive impact on the circadian rhythm of skin temperature in the 12 patients with evaluable data. Not to be bound by theory, it is believed that aminosterols could be affecting neuronal circuits involving the master clock (the suprachiasmatic nucleus) and its autonomic projections and opens the possibility of therapeutic correction of circadian dysfunction.

As described in Example 8, aminosterol dosing is patient specific, as the dose is likely related to the extent of neuronal damage, with greater neuronal damage correlating with the need for a higher aminosterol dose to obtain a desired therapeutic result. As described in greater detail herein, aminosterol dosing can range from about 0.01 to about 500 mg/day, with dosage determination described in more detail below.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

### I. Deuterated Forms of Aminosterols

In one aspect, provided herein are deuterated forms of aminosterols, or a pharmaceutically acceptable salt thereof.

As used herein, unless otherwise indicated, carbon positions in the aminosterol are numbered as follows in the following chemical structure:

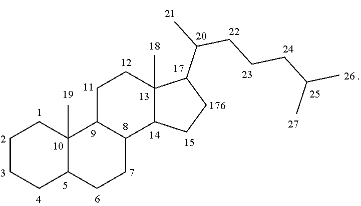 [SMILES: CC(C)CCCC(C)C1CCC2C3CCC4CCCCC4(C)C3CCC12C]

The structural formula depicted herein may or may not indicate whether atoms at certain positions are isotopically enriched. In a most general embodiment, unless otherwise indicated, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, unless otherwise indicated, the stable isotopes are present at natural abundance at all positions in a compound not specifically designated as being isotopically enriched.

The compounds of the present technology may comprise an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436 or a salt or derivative thereof, squalamine or a salt or derivative thereof, or a combination thereof.

In some embodiments, the aminosterol is squalamine or a salt or derivative thereof. In some embodiments, the aminosterol is Aminosterol 1436 or a salt or derivative thereof.

In some embodiments, the aminosterol is a derivative of squalamine, or a derivative of aminosterol 1436, modified through medical chemistry to improve biodistribution, ease of administration, metabolic stability, or any combination thereof. In some embodiments, squalamine or aminosterol 1436 is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In some embodiments, disclosed herein are compounds having the structure of

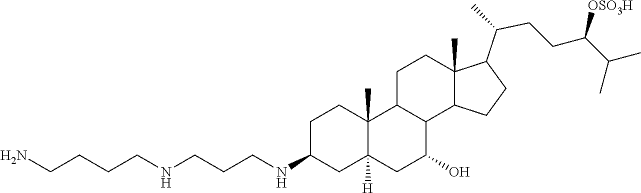

squalamine, or a pharmaceutically acceptable salt thereof, or

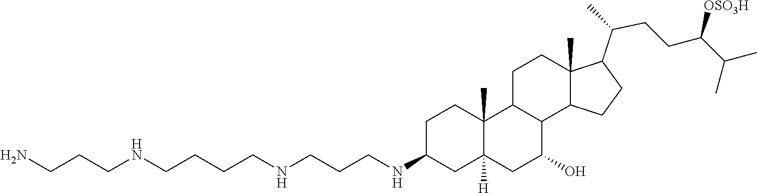

aminosterol 1436, or a pharmaceutically acceptable salt thereof, or

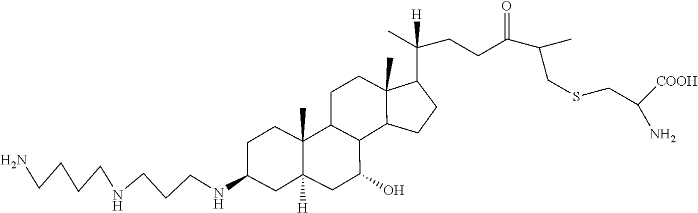 [SMILES: CC(CSCC(N)C(=O)O)C(=O)CC[C@@H](C)C1CCC2C3C(CC[C@@]21C)[C@@]1(C)CC[C@H](NCCCNCCCCN)C[C@@H]1C[C@H]3O]

Compound 1, or a pharmaceutically acceptable salt thereof, or

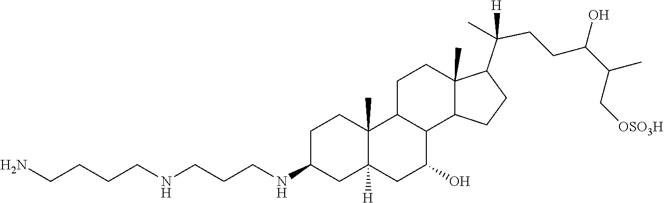

Compound 2, or a pharmaceutically acceptable salt thereof, or

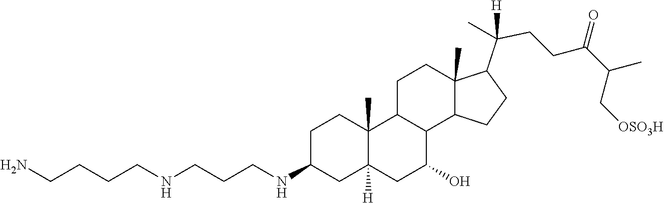

Compound 3, or a pharmaceutically acceptable salt thereof, or

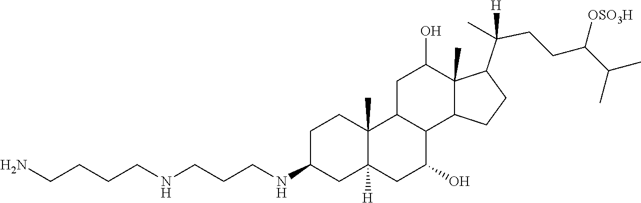

Compound 4, or a pharmaceutically acceptable salt thereof, or

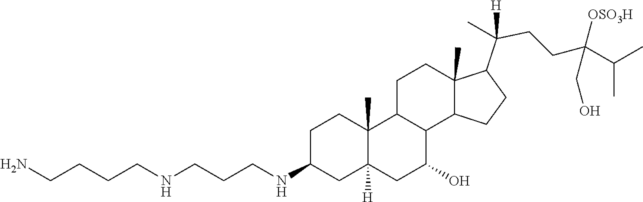

Compound 5, or a pharmaceutically acceptable salt thereof, or

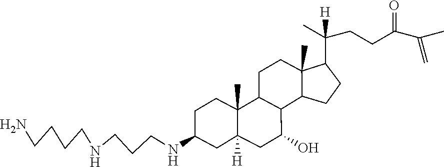 [SMILES: C=C(C)C(=O)CC[C@@H](C)C1CCC2C3C(CC[C@@]21C)[C@@]1(C)CC[C@H](NCCCNCCCCN)C[C@@H]1C[C@H]3O]

Compound 6, or a pharmaceutically acceptable salt thereof,
   wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium.

In some embodiments, one or more hydrogen atoms at one or more positions selected from C3, C4, C5, C7, C17, C20, C21, C22, C23, C24, C25, C26, and C27, are replaced with deuterium. In some embodiments, one or more hydrogen atoms at one or more positions selected from C3, C4, C5, C7, C22, C23, C24, C25, C26, and C27, are replaced with deuterium. In some embodiments, one or more hydrogen atoms at one or more positions selected from C4, C5, C7, C22, C23, C24, C25, C26, and C27, are replaced with deuterium.

In some embodiments, all hydrogen atoms at positions C25, C26, and C27 are replaced with deuterium. In some embodiments, all hydrogen atoms at positions C26 and C27 are replaced with deuterium.

In some embodiments, one or more hydrogen atoms at one or more positions selected from C1, C2, C3, C4, C5, C6, C7, C8, C9, C11, C12, C14, C15, C16, C17, C18, and C19, are replaced with deuterium.

In some embodiments, disclosed herein is a compound selected from:

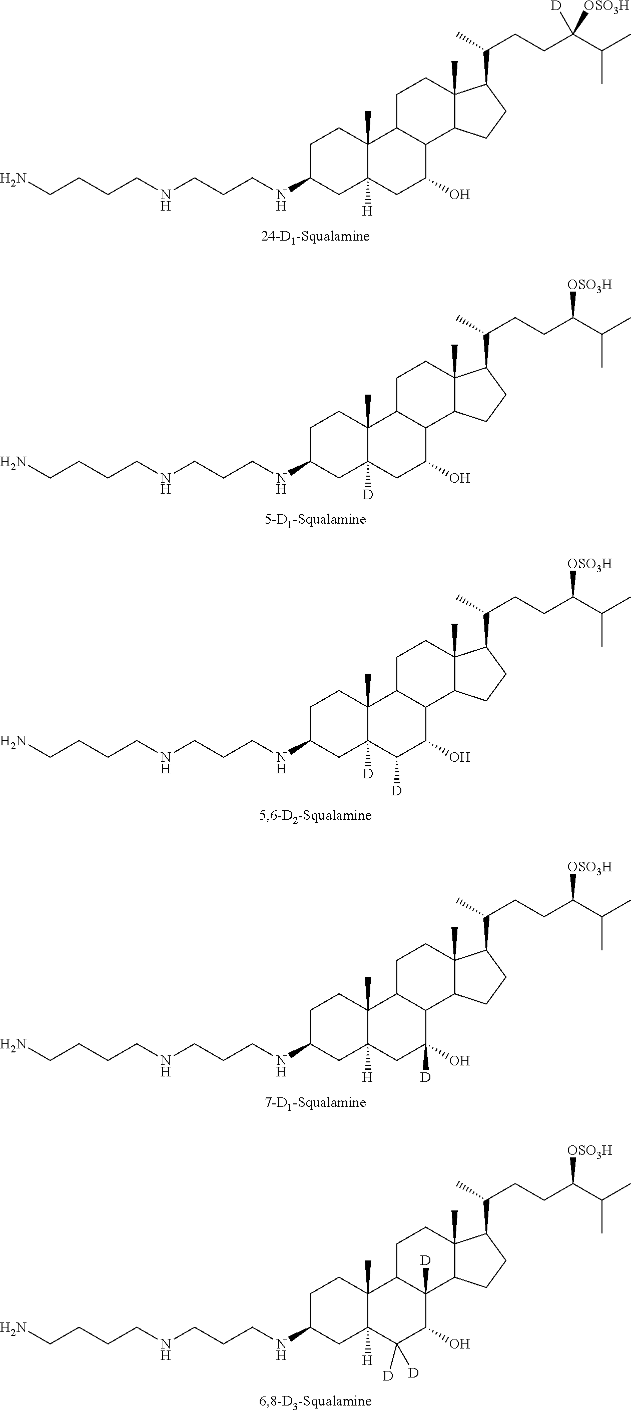

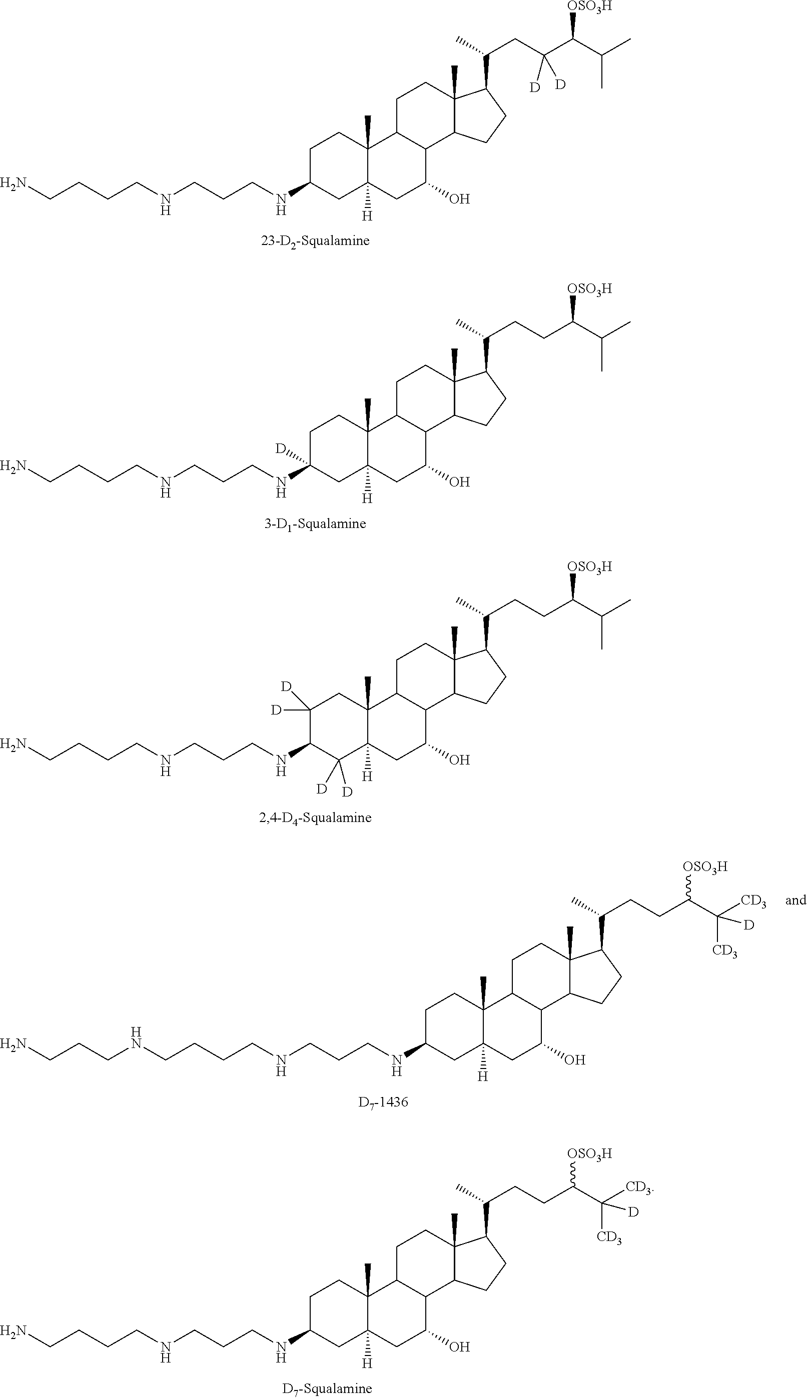

The present technology also provides salts, solvates and hydrates of the compounds disclosed herein.

A salt of a compound of this technology is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, the compositions used in the methods of the invention comprise: (a) at least one pharmaceutical grade deuterated aminosterol; and optionally (b) at least one phosphate selected from the group consisting of an inorganic phosphate, an inorganic pyrophosphate, and an organic phosphate. In some embodiments, the deuterated aminosterol is formulated as a weakly water-soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosutfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-I,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, 3-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid, hydrobromic acid, and phosphoric acid.

### II. Compositions

In another aspect, provided herein are compositions comprising a deuterated form of an aminosterol, or a pharmaceutically acceptable salt thereof, and one or more pharmaceutically acceptable carriers and/or excipients.

In some embodiments, provided herein are pharmaceutical compositions comprising, consisting essentially of, or consisting of a deuterated form of squalamine, or a pharmaceutically acceptable salt thereof, and one or more pharmaceutically acceptable carriers and/or excipients.

In some embodiments, provided herein are pharmaceutical compositions comprising, consisting essentially of, or consisting of a deuterated form of Aminosterol 1436, or a pharmaceutically acceptable salt thereof, and one or more pharmaceutically acceptable carriers and/or excipients.

### A. Pharmaceutical Carriers

While it is possible for a deuterated form of an aminosterol, or a pharmaceutically acceptable salt thereof, to be administered alone, it is preferable to present it as a pharmaceutical formulation, together with one or more acceptable carriers. The carrier(s) must be “acceptable” in the sense of being compatible with a deuterated form of an aminosterol, or a pharmaceutically acceptable salt thereof, and not deleterious to the recipients thereof.

Generally, the formulations are prepared by contacting a deuterated form of an aminosterol, or a pharmaceutically acceptable salt thereof, uniformly and intimately with liquid carriers or finely divided solid carriers or both. Then, if necessary, the product is shaped into the desired formulation. Non-aqueous vehicles such as fixed oils and ethyl oleate are also useful herein, as well as liposomes.

The carrier suitably comprises minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as gelatin, serum albumin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

In instances where aerosol administration is appropriate, a deuterated form of an aminosterol, or a pharmaceutically acceptable salt thereof, can be formulated as aerosols using standard procedures. The term “aerosol” includes any gas-borne suspended phase of a compound described herein which is capable of being inhaled into the bronchioles or nasal passages, and includes dry powder and aqueous aerosol, and pulmonary and nasal aerosols. Specifically, aerosol includes a gas-born suspension of droplets of a compound described herein, as may be produced in a metered dose inhaler or nebulizer, or in a mist sprayer. Aerosol also includes a dry powder composition of a composition of the present technology suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al., Pharmacol. Toxicol. Methods, 27:143-159 (1992).

### B. Dosage Forms

The deuterated aminosterol formulations may conveniently be presented in unit dosage form and may be prepared by any of the methods well known in the art of pharmacy. Exemplary deuterated aminosterol dosage forms include, but are not limited to, oral, intranasal, and injectable (IP, IV, or IM). Preferably, the deuterated aminosterol formulation is administered orally, intranasally, or a combination thereof.

Formulations or compositions of the present technology may be packaged together with, or included in a kit with, instructions or a package insert.

“Pharmaceutically acceptable carrier” refers to a non-toxic solid, semisolid or liquid filler, diluent, encapsulating material or formulation auxiliary of any type.

Pharmaceutical compositions according to the present technology may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, may include colloidal silicon dioxide, such as Aerosil® 200, talc, stearic acid, magnesium stearate, calcium stearate, and silica gel.

Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

### C. Dosages & Dosing Period

Oral dosage of a deuterated aminosterol can range from about 1 to about 500 mg/day, or any amount in-between these two values. Other exemplary dosages of orally administered deuterated aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of a deuterated aminosterol are much lower than oral dosages of a deuterated aminosterol. Examples of such intranasal deuterated aminosterol low dosages include, but are not limited to, about 0.001 to about 6 mg, or any amount in-between these two values. For example, the low dosage of an intranasal administered deuterated aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the deuterated aminosterol dosage may be selected such that it would not provide any pharmacological effect if administered by any other route and, in addition, does not result in negative effects. For example, Aminosterol 1436 is known to have the pharmacological effects of a reduction in food intake and weight loss.

Therefore, in the IN methods of the invention, if the deuterated aminosterol is deuterated Aminosterol 1436 or a salt or derivative thereof, then if the IN deuterated Aminosterol 1436 dosage is administered via another route, such as oral, IP, or IV, then the deuterated Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the deuterated aminosterol is deuterated squalamine or a salt or derivative thereof, then if the IN deuterated squalamine dosage is administered via another route, such as oral, IP, or IV, then the deuterated squalamine dosage will not result in noticeable nausea, vomiting, and/or a reduction in blood pressure.

Deuterated aminosterol doses can be de-escalated (reduced) if any given deuterated aminosterol dose induces a persistent undesirable side effect, such as diarrhea, vomiting, or nausea. In another embodiment, a dose of a deuterated aminosterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment a deuterated aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

The pharmaceutical composition comprising a deuterated aminosterol or a derivative or salt thereof can be administered for any suitable period of time, including as a maintenance dose for a prolonged period of time. Dosing can be done on an as needed basis using any pharmaceutically acceptable dosing regimen. Deuterated aminosterol dosing can be no more than 1x per day, once every other day, once every three days, once every four days, once every five days, once every six days, once a week, or divided over multiple time periods during a given day (e.g., twice daily).

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the deuterated aminosterol dose is taken in the morning, i.e. on an empty stomach preferably within about two hours of waking up and may be followed by a period without food, such as for example about 60 to about 90 minutes. In other embodiments, the deuterated aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the deuterated aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the invention, the aminosterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

### III. Methods of Prevention and/or Treatment with Deuterated Aminosterols

Aspects of this disclosure relate to methods of treating certain symptoms and/or methods of treating and/or preventing disorders associated with one or more of these symptoms by administration of an effective amount of a deuterated aminosterol, or a pharmaceutically acceptable salt thereof, in one or more pharmaceutically acceptable carriers. The compositions of the present technology can be administered using any pharmaceutically acceptable method, including but not limited to oral, pulmonary, nasal, and nebularization administration.

The deuterated aminosterol compositions of the present technology can be used to treat any indication known to be amenable to treatment with an aminosterol. Accordingly, in some embodiments, provided herein are methods for treating a subject in need having a condition susceptible to treatment with an aminosterol, comprising administering to the subject a therapeutically effective amount of a deuterated form of an aminosterol, or a pharmaceutically acceptable salt thereof. In some embodiments, provided herein are methods for treating a subject in need having a condition susceptible to treatment with an aminosterol, comprising administering to the subject a therapeutically effective amount of a composition comprising or consisting essentially of a deuterated form of an aminosterol, or a pharmaceutically acceptable salt thereof, and one or more pharmaceutically acceptable carriers and/or excipients.

The deuterated aminosterol compositions of the present technology can be used to treat, for example, (1) disorders of gastrointestinal motility, such as chronic idiopathic constipation, opioid induced constipation, irritable bowel syndrome, and inflammatory bowel disease; (2) hallucinations or related symptoms; (3) sleep disturbances and/or sleep problems, such as REM disturbed sleep or circadian rhythm disfunction, or a related symptom; (4) cognitive impairment or a related symptom; (5) depression; (6) a condition or symptom associated with alpha-synuclein aggregation; (7) neurological diseases or conditions that could benefit from neuro-protection, or a related symptom, such as Parkinson's Disease, Alzheimer's disease, Huntington's Disease, multiple system atrophy, schizophrenia, autism, Progressive supranuclear palsy, Frontotemporal dementia (FTD), vascular dementia, Amyotrophic lateral sclerosis (ALS), multiple sclerosis (MS), spinal muscular atrophy (SMA), Friedreich's ataxia, acute traumatic injury to the central nervous system, including the spinal cord, stroke, acute head and/or spine injury, degenerative processes associated with aging, including memory loss (“dementia of aging”), cerebral palsy, epilepsy, peripheral sensory neuropathy, and autonomic system lability; (8) viral infections; (9) antimicrobial infections, including but not limited to treating and/or preventing Gram-negative and Gram-positive bacterial infections, fungal infections, and protozoan infections; (10) disease states known to be associated with pathological neovascularization, such as cancer; (11) vascular disorders of the eye, including macular degeneration, such as age-related macular degeneration, retinopathy of prematurity, corneal neovascularization, diabetic retinopathy; (12) weight loss or weight management, dose-dependent weight loss; (13) treatment of fibrodysplasia ossificans progressiva, a rare disease where connective tissue will ossify when damaged; and (14) diabetes mellitus and diabetic neuropathy.

### A. Gastrointestinal Motility Disorders

In one aspect of the invention, encompassed are methods of treating gastrointestinal motility disorders, such as chronic idiopathic constipation, opioid induced constipation, irritable bowel syndrome, and inflammatory bowel disease, or a related symptom, comprising administering to a subject in need a therapeutically effective amount of at least one deuterated aminosterol or a pharmaceutically acceptable salt thereof.

Constipation is a common problem worldwide, affecting 2% to 27% of the population, with most estimates varying from 12% to 20%. The prevalence of constipation increases to 30%-40% among people aged >65 years and women are disproportionately affected. In North America, 63M people meet the Rome IV criteria for constipation and in the US alone, constipation is responsible for over 2M physician visits annually. Laxatives are prescribed to 2-3M patients every year and furthermore, in most patients, the condition is chronic requiring life-long treatment. This represents an economic burden to the individual with constipation and to the healthcare system. According to the most recent Federal Supply Schedule (FSS; April 2016), the average 30-day reimbursed price for a basket of orally administered drugs for constipation is approximately $260 or $3120 per year.

Constipation not only constitutes a major economic burden, but it also significantly affects the quality of life of the individual, contributing to social isolation and depression. Furthermore, the severity of the symptoms correlates negatively with patient reported quality of life.

An effective pro-kinetic medication for individuals with constipation would be a useful addition to the currently available treatments for this condition.

Constipation is defined as a lower than normal frequency of bowel movements in a fixed duration of time (e.g. less than 3 bowel movements per week). While often dismissed as strictly a gastrointestinal symptom, constipation is believed to be an early indicator of neurodegenerative disease to the extent that ENS degeneration can be indicative of later CNS degeneration. Indeed, not to be bound by theory, but constipation is believed to be one of the earliest indicators of Parkinson's Disease pathology. Accordingly, method embodiments disclosed herein relate to the treatment of constipation or the treatment and/or prevention of an underlying disorder associated with constipation.

Constipation is common in PD and often becomes symptomatic years before the onset of the motor dysfunction and the subsequent diagnosis of PD. There is substantial evidence that the neurodegenerative process associated with PD, namely the accumulation of toxic aggregates of alpha-synuclein, occurs within the enteric nervous system years before they appear within the brain. It is believed that the enteric nervous system (ENS), with its vast surface area, is subject to continuous insults from infectious agents and toxic substances. Although the function of alpha-synuclein is not known, inflammation within the nervous system leads to an increase in its intracellular levels. In individuals with PD the increase in alpha-synuclein leads to the formation of neurotoxic aggregates, perhaps because of a failure by the neuron (due to genetic factors) to effectively dispose of them. The aggregates of alpha-synuclein then traffic along the vagal nerve to the dorsal motor nucleus within the brainstem, and from there to more rostral structures.

Several tools can be used to measure and evaluate the effect of deuterated aminosterol treatment on constipation, as detailed in Example 8, including for example:

(1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(2) Constipation—Ease of Evacuation Scale (from 1-7, with 7=incontinent, 4=normal, and 1=manual disimpaction);

(3) Bristol Stool Chart, which is a patient-friendly means of categorizing stool characteristics (assessment of stool consistency is a validated surrogate of intestinal motility) and stool diary;

(4) Unified Parkinson's Disease Scale (UPSRS), section 1.11 (Constipation Problems);

(5) Patient Assessment of Constipation Symptoms (PAC-SYM); and

(5) Patient Assessment of Constipation Quality of Life (PAC-QOL).

Examples of characteristics of constipation that can be positively affected by the method of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the invention. Further, assessments of these characteristics are known in the art, e.g. spontaneous bowel movements (SBMs)/week, stool consistency (Bristol Stool Form Scale) (Lewis and Heaton 1997; Heaton et al. 1992), ease of passage (Ease of Evacuation Scale) (Andresen et al. 2007), rescue medication use and symptoms and quality of life related to bowel function (PAC-SYM (Frank et al. 1999) and PAC-QOL (Marquis et al. 2005)).

The methods of using a therapeutically effective amount of a deuterated aminosterol composition according to the invention to treat and/or prevent constipation preferably results in an increase in the number of spontaneous bowel movements per week and/or an improvement in other stool conditions. The increase can be, for example, an increase of between 1 to 3 spontaneous bowel movements in a week, or, optionally, full restoration of regular bowel function.

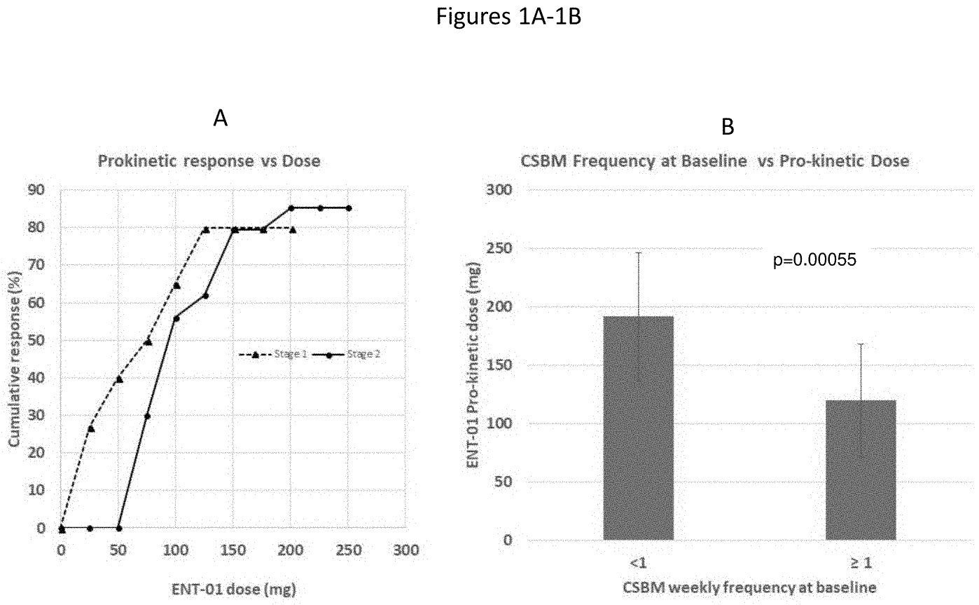

Data detailed in Example 8 shows that 80% of subjects responded to aminosterol treatment with improved bowel function (see FIG. 1A), with the cumulative response rate increasing in a dose-dependent fashion from 25% at 25 mg to a maximum of 80% at 200 mg (Stage 1, FIG. 1A). In Stage 2 of the study, the response rate increased in a dose-dependent fashion from 26% at 75 mg to 85.3% at 250 mg (FIG. 1A). The dose required for a bowel response was patient-specific and varied from 75 mg to 250 mg. The median efficacious dose was 100 mg.

The average CSBM/week increased from 1.2 at baseline to 3.8 at fixed dose (216% improvement) and SBM increased from 2.6 at baseline to 4.5 at fixed dose (73% improvement). Use of rescue medication decreased from 1.8/week at baseline to 0.3 at fixed dose (83% decrease). Consistency based on the Bristol stool scale also improved, increasing from mean 2.7 to 4.1 (52% improvement) and ease of passage increased from 3.2 to 3.7 (16% improvement). Subjective indices of wellbeing (PAC-QOL) and constipation symptoms (PAC-SYM) also improved during treatment.

The dose that proved efficacious in inducing a bowel response was strongly related to constipation severity at baseline (FIG. 1B); patients with baseline constipation of <1 CSBM/week required higher doses for a response (mean 192 mg) than patients with ≥1 CSBM/week (mean 120 mg).

In one embodiment of the invention, treatment of a subject having constipation with a deuterated aminosterol in a method described herein results in an improvement of one or more characteristics of constipation. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%. Examples of constipation characteristics that can be improved by the methods of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Measurement of a constipation characteristic can be done using any clinically recognized scale or tool.

In one embodiment, the dose of deuterated aminosterol correlates with the severity of the constipation or related symptom, with more severe constipation, defined as a baseline rate of less than one CSBM or SBM per week in the subject, requiring a higher dose of a deuterated aminosterol, as compared to moderate constipation, defined as a baseline rate of one or more CSBM or SBM per week in the subject. It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of aminosterol required to restore normal bowel function. Moreover, the data described in Example 8 confirms the hypothesis that gastrointestinal dysmotility in PD results from the progressive accumulation of αS in the ENS, and that aminosterol treatment can restore neuronal function by displacing αS and stimulating enteric neurons. These results demonstrate that the ENS in PD is not irreversibly damaged and can be restored to normal function.

For example, a subject experiencing severe constipation or a related symptom could be administered a daily deuterated aminosterol oral dose ranging from about 75 mg up to about 500 mg, or any amount in-between these two values as described herein. A positive effect in such a method can be a dose that results in a CSBM within 24 hours of dosing on at least 2 of 3 days at a given dose. In addition, an oral deuterated aminosterol dose for a mild or moderately constipated patient could range, for example, from about 5 mg up to about 350 mg, or any amount in-between these two values as described herein.

### B. Hallucinations

In one aspect of the invention, encompassed are methods of treating hallucinations or a related symptom comprising administering to a subject in need a therapeutically effective amount of at least one deuterated aminosterol or a pharmaceutically acceptable salt thereof.

A hallucination is a sensory impression or perception of an object or event, in any of the 5 senses (sight, touch, sound, smell, or taste) that has no basis in external stimulation. Hallucinations can have debilitating impact on the subject's health and life by causing harm to self or others, by making it difficult for the subject to function normally in everyday situations, and by causing sleep disruption. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination a hallucination involving the perception of a physical experience occurring within the body.

Hallucinations can be a result of psychiatric conditions or correlated with diseases, such as a neurodisease. Hallucinations, especially auditory hallucinations, are characteristic of certain psychiatric conditions such as schizophrenia, occurring in up to 70-80% of subjects. They also occur in 30-50% of individuals with borderline personality disorder. Auditory hallucinations can take control of actions or behavior and elicit violent defensive behavior or alternatively lead to self-harming behavior. They can also occur in post-partum psychosis. Auditory hallucinations can less commonly occur in severely depressed patients or even in mania. Substance abuse can also be associated with visual hallucinations. Alcohol intoxication or withdrawal, post-traumatic stress disorder (PTSD) and bereavement can also be associated with visual hallucinations.

Hallucinations can be a result of neurological disorders. In one embodiment the neurological disorder is a brain tumor. In some embodiments, the “focal brain lesions.” Formed and unformed visual hallucinations can occur in the presence of temporal and occipital lobe lesions. Occipital lobe lesions typically produce simple geometric patterns or “strings of circles like a bunch of grapes” or stars which can follow the gaze (palinopsia), whereas temporal lobe lesions are associated with complex, formed hallucinations. Temporal lobe lesions and especially lesions of the uncinate gyrus are typically associated with olfactory and gustatory hallucinations. Lesions of the cerebral peduncles and substantia nigra are associated with “peduncular hallucinosis” or colorful vivid images. In some embodiments, the hallucinations are a result of diffuse involvement of the cerebral cortex. In some embodiments, of diffuse involvement. Acute metabolic encephalopathies and encephalitis caused by viral infections or diseases associated with a cerebral vasculitis such as Systemic Lupus Erythematosus (SLE) can cause visual hallucinations.

In some cases, hallucination is the result of a psychiatric or neurological disorder. The aminosterol composition can, for example, reverse the dysfunction of the psychiatric or neurological disorder and treat the hallucination. The psychiatric disorder can be, for example, selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia.

In other cases, hallucinations can be the result of a neurological disorder. The neurological disorder can be, for example, the result of (a) a brain tumor, (b) a sleep disorder such as narcolepsy, or (c) a focal brain lesion, such as occipital lobe lesions or temporal lobe lesions. In an exemplary embodiment, the temporal lobe lesion can be lesions of the uncinate gyrus, cerebral peduncles, or substantia nigra. The neurological disorder can be, for example, the result of (d) a diffuse involvement of the cerebral cortex, such as that caused by a viral infectious disease.

The diffuse involvement of the cerebral cortex can be a result of a cerebral vasculitis condition, and the viral infectious disease can be, for example, acute metabolic encephalopathies, encephalitis, or meningitis. The cerebral vasculitis condition can be caused by an autoimmune disorder, a bacterial or viral infection, or a systemic vasculitis. The autoimmune disorder can be, for example, Systemic Lupus Erythematosus (SLE).

Alternatively, hallucinations can be the result of a neurodegenerative disorder. For example, the neurodegenerative disorder can be, for example, such as Parkinson's disease (PD), supranuclear palsy, multi-system atrophy, Parkinsonism, Alzheimer's disease, Fronto-temporal dementia, amyotrophic lateral sclerosis (ALS), Huntington's Disease, schizophrenia, Friedreich's ataxia, Multiple sclerosis (MS), Lewy Body dementia or disease, spinal muscular atrophy, fronto temperal dementia, progressive nuclear palsy, Guadeloupian Parkinsonism, spinocerebellar ataxia, or vascular dementia. In a preferred embodiment, the aminosterol compositions of the invention reverse the dysfunction of the neurodegenerative disorder and treat the hallucination.

Further still, hallucinations may be caused by a sensory loss. The sensory loss can be, for example, visual, auditory, gustatory, tactile, or olfactory. In a preferred embodiment, the fixed dose aminosterol compositions of the invention reverse the dysfunction of the sensory loss and treat the hallucination. In a preferred embodiment, the aminosterol compositions of the invention reverse the dysfunction of the enteric nervous system and treats the hallucination.

The methods of using a therapeutically effective amount of a deuterated aminosterol composition according to the invention to treat and/or prevent hallucinations preferably result in a decrease in hallucinations. The decrease can be, for example, a reduction in occurrences of hallucinations by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The methods of the invention may also result in the subject being hallucination-free. The hallucination can comprise, for example, a visual, auditory, tactile, gustatory or olfactory hallucination. The improvement can be measured using any clinically recognized assessment or tool.

Several tools can be used to measure and evaluate the effect of deuterated aminosterol treatment on hallucinations, such as detailed in Example 8, including for example:

(1) The University of Miami Parkinson's Disease Hallucinations Questionnaire (UM-PDHQ);

(2) Unified Parkinson's Disease Scale (UPSRS), section 1.2 (Hallucinations and Psychosis); and

(3) direct questioning.

As described in Example 8, the PDHQ score improved from 1.3 at baseline to 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg. Further, unlike stool-related indices, the improvement in many CNS symptoms persisted during wash-out.

### C. Sleep Disturbance/Sleep Problems

### (e.g., REM Disturbed Sleep or Circadian Rhythm Dysfunction)

In another aspect of the invention, encompassed are methods of treating sleep disturbances and/or sleep problems, or a related symptom, comprising administering to a subject in need a therapeutically effective amount of at least one deuterated aminosterol or a pharmaceutically acceptable salt thereof.

Normal sleep is critically important for the proper functioning of many organ systems, the most important of which is the brain. Disturbances in normal sleep patterns are closely associated with the normal aging process, with the development of cognitive impairment, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “zeitgebber” or circadian clock. These conditions may be reversible, such as desynchronization resulting from jet-lag, night-shift work or hunger, conditions easily remedied by adaptation or food intake. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to REM-behavior disorders, disturbances in the Circadian rhythm, delayed sleep onset, sleep fragmentation, and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” (RBD) or “REM-disturbed sleep”, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

Sleep disturbances include but are not limited to RBD, circadian rhythm dysfunction, delayed sleep onset, Restless leg syndrome, daytime sleepiness, and sleep fragmentation.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

A “normal” or “restful” sleep period is defined as a sleep period uninterrupted by wakefulness. Alternatively, a said period can be defined by the recommended or appropriate amount of sleep for the subject's age category, e.g., (i) infants 0-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults ≥65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008. Similarly symptoms of RBD can be monitored using a daily diary and RBD questionnaire (Stiasny-Kolster et al. 2007).

In some embodiments, administration of a therapeutically effective amount of a deuterated aminosterol composition to a patient with disturbed sleep results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

There are several tools that can used to measure and evaluate the effect of aminosterol treatment on sleep, as detailed in Example 8, including for example:

(1) Sleep Diary (participants completed a sleep diary on a daily basis throughout the study. The diaries included time into bed and estimated time to sleep as well as wake time and duration during the night.);

(2) I-Button Temperature Assessment. The I-Button is a small, rugged self-sufficient system that measures temperature and records the results in a protected memory section. The Thermochron I-Button DS 1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(3) Unified Parkinson's Disease Rating Scale (UPDRS), sections 1.7 (sleep problems), 1.8 (daytime sleepiness) and 1.13 (fatigue);

(4) Parkinson's Disease Fatigue Scale (PFS-16);

(5) REM Sleep Behavior Disorder Screening Questionnaire; and

(6) Parkinson's Disease Sleep Scale.

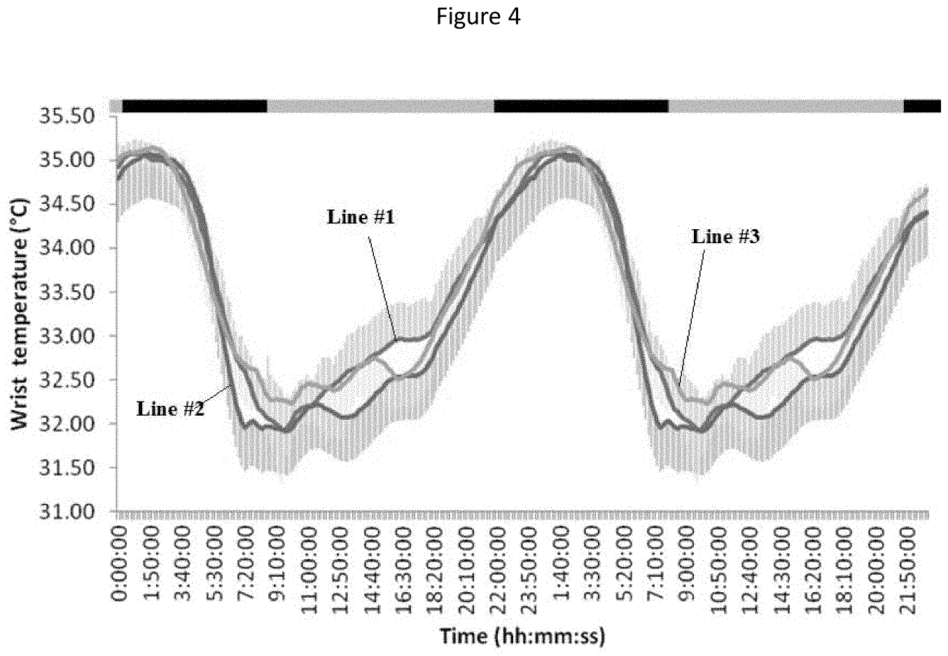

The data detailed in Example 8 described how circadian system status was evaluated by continuously monitoring wrist skin temperature (Thermochron iButton DS 1921H; Maxim, Dallas) following published procedures (Sarabia et al. 2008). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIG. 4). Unlike stool-related indices, the improvement in many CNS symptoms persisted during wash-out.

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm.

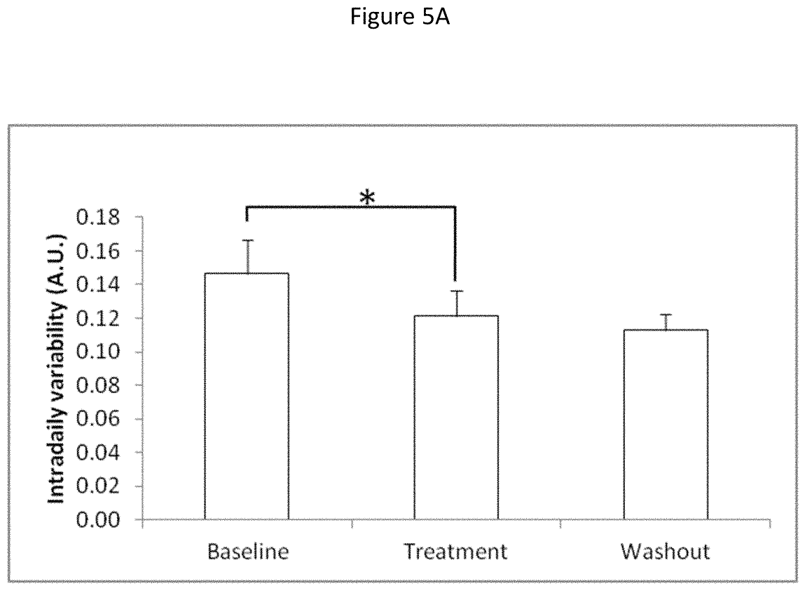

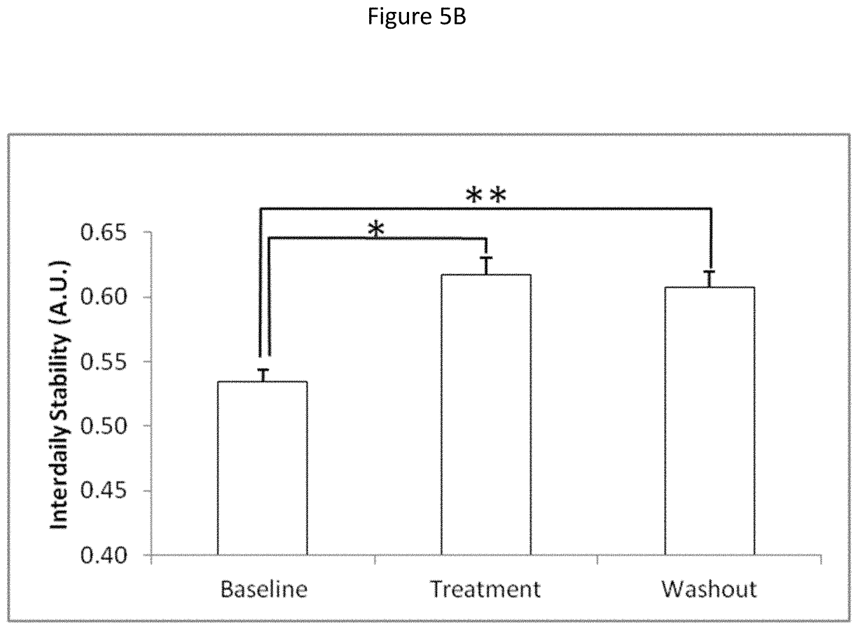

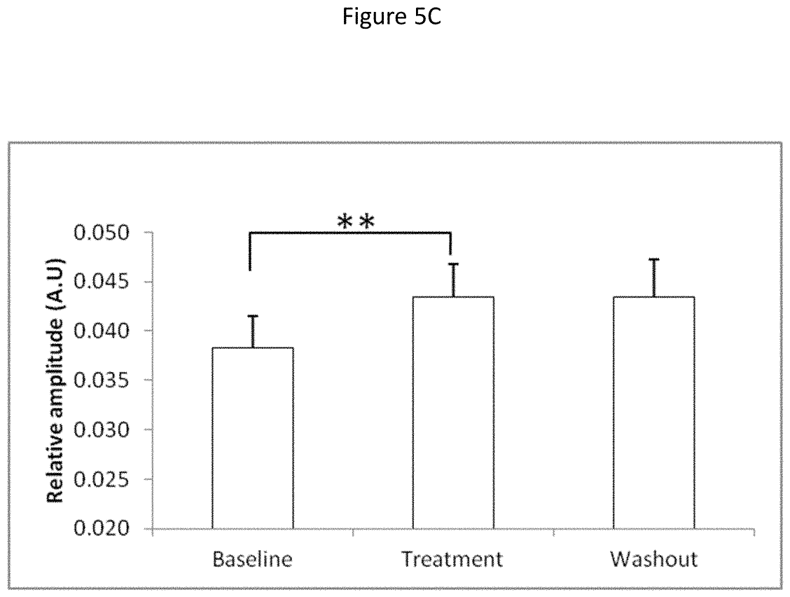

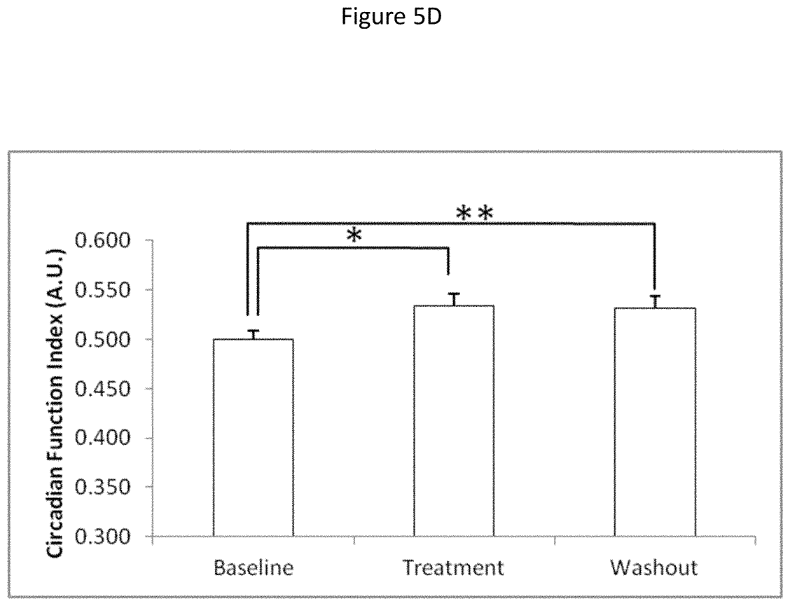

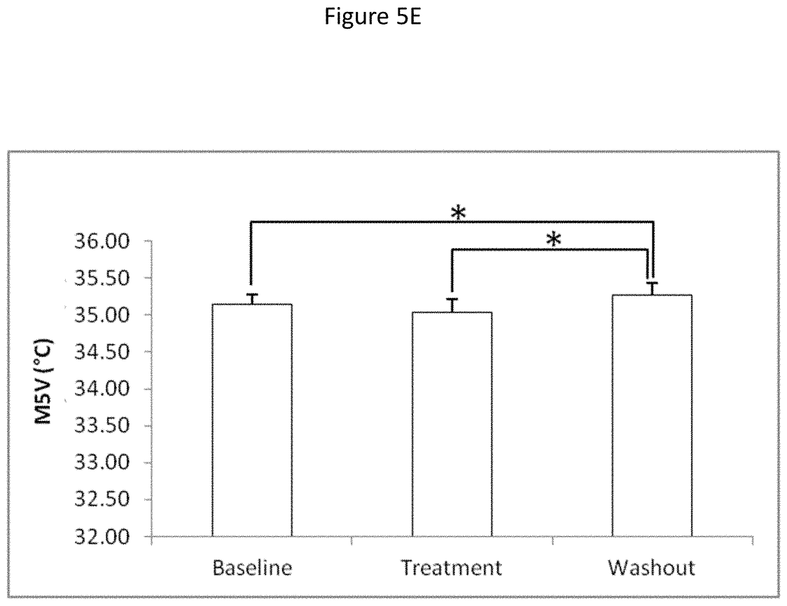

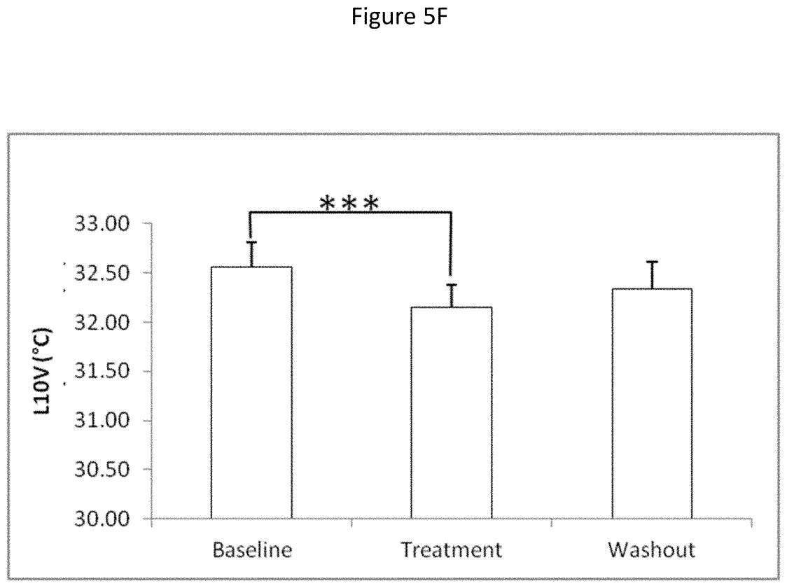

A comparison was performed of circadian rhythm parameters during the baseline, fixed dose and washout periods. Aminosterol administration improved all markers of healthy circadian function, including increasing rhythm stability, relative amplitude, and circadian function index, while reducing rhythm fragmentation. The improvement persisted for several of these circadian parameters during the wash-out period. (FIG. 5). Improvements were also seen in REM-behavior disorder (RBD) and sleep. RBD and total sleep time also improved progressively in a dose-dependent manner.

### D. Cognitive Impairment

In another aspect of the invention, encompassed are methods of treating cognitive impairment or a related symptom comprising administering to a subject in need a therapeutically effective amount of at least one deuterated aminosterol or a pharmaceutically acceptable salt thereof.

Cognitive impairment, including mild cognitive impairment (MCI), is characterized by increased memory or thinking problems exhibited by a subject as compared to a normal subject of the same age. Approximately 15 to 20 percent of people age 65 or older have MCI, and MCI is especially linked to neurodegenerative conditions such as Alzheimer's disease (AD) or synucleopathies like Parkinson's disease (PD). In 2002, an estimated 5.4 million people (22%) in the United States over age 70 had cognitive impairment without dementia. Plassman et al. 2009.

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Related disorders and conditions include, but are not limited to, dementia, Alzheimer's, delirium, Parkinson's, diabetes, high blood pressure, high cholesterol, depression, psychological and behavioral conditions, amnesia, Lewy body diseases, or Huntington's disease, among others.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al. 2011; McKhann et al. 2011; Albert et al. 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used. Palsetia et al. (2018); Kirkevold, O. & Selbaek, G. (2015). With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F. 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective amount of a deuterated aminosterol composition to a patient in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Cognitive impairment and the improvement following deuterated aminosterol treatment can be assessed using several tools, as detailed in Example 8:

(1) Mini Mental State Examination (MMSE);

(2) Trail Making Test (TMT) Parts A and B; and

(3) Unified Parkinson's Disease Rating Scale (UPDRS), sections 1.1 (cognitive impairment).

Assessments were made at baseline and at the end of the fixed dose and washout periods for Example 8, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.1, cognitive impairment) had a mean baseline score of 11.6, a fixed aminosterol dose mean score of 10.6, and a wash-out mean score of 9.5, demonstrating an almost 20% improvement (UPDRS cognitive impairment is rated from 1=slight improvement to 4=severe impairment, so lower scores correlate with better cognitive function). In addition, MMSE improved from 28.4 at baseline to 28.7 during treatment and to 29.3 during wash-out (the MMSE has a total possible score of 30, with higher scores correlating with better cognitive function). Unlike stool-related indices, the improvement in many CNS symptoms persisted during wash-out.

### E. Depression

In another aspect of the invention, encompassed are methods of treating depression or a related symptom comprising administering to a subject in need a therapeutically effective amount of at least one deuterated aminosterol or a pharmaceutically acceptable salt thereof.

Clinical depression is characterized by a sad, blue mood that goes above and beyond normal sadness or grief. Major depression is an episode of sadness or apathy along with other symptoms that lasts at least two consecutive weeks and is severe enough to interrupt daily activities. Depressive events feature not only negative thoughts, moods, and behaviors but also specific changes in bodily functions (like, eating, sleeping, energy and sexual activity, as well as potentially developing aches or pains). One in 10 people will have a depression in their lifetime. Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the working brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the invention, encompassed are methods of treating and/or preventing depression comprising administering therapeutically effective amount of a deuterated aminosterol composition according to the invention. While not wishing to be bound by theory, it is theorized that the deuterated aminosterol compositions of the invention trigger neurogenesis, which functions to combat depression.

In some embodiments, the methods of the invention produce an improvement in a subject's clinical depression. An improvement in a subject's depression can be measured using any clinically-recognized measurement. For example, improvement can be measured using a depression rating scale. In one embodiment of the invention, following treatment a subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement. The improvement can be measured using any clinically recognized tool or assessment.

Depression and/or mood and the improvement following deuterated aminosterol treatment can be assessed using any clinically recognized tool, as detailed in Example 8, such as:

(1) Beck Depression Inventory (BDI-II);

(2) Unified Parkinson's Disease Rating Scale (UPDRS), sections 1.3 (depressed mood), 1.4 (anxious mood), 1.5 (apathy), and 1.13 (fatigue); and

(3) Parkinson's Disease Fatigue Scale (PFS-16).

### F. Alpha-Synuclein Aggregation

In another aspect of the invention, encompassed are methods of treating a condition or symptom associated with alpha-synuclein aggregation comprising administering to a subject in need a therapeutically effective amount of at least one deuterated aminosterol or a pharmaceutically acceptable salt thereof.

Alpha-synuclein is a potent pro-inflammatory hormone. Inflammation can be blocked by either of two strategies. First, inflammation can be blocked by reducing the tissue concentration of alpha-synuclein by decreasing or stopping production of alpha-synuclein. Alternatively, inflammation can be blocked by interrupting the signaling between alpha-synuclein and inflammatory cells that express CD11b. The subject of the methods of the invention can be any mammal, including a human.

The inflammatory disease or condition caused by excessive expression of neuronal alpha synuclein can be a neurodegenerative disorder (NDD), such as an alpha-synucleinopathy. Exemplary alpha-synucleinopathies include, but are not limited to, PD, Lewy body dementia, multiple system atrophy, amytrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal alpha synuclein can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases.

The methods of the invention can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases. A still further example is a patient population having elevated levels of alpha-synuclein aggregation in their enteric nerve cells, manifesting as a constipation.

In addition, it follows from the present invention that an individual with an inflammatory condition appropriate for treatment or prophylaxis with the methods targeting alpha-synuclein described herein can be identified by determination of the tissue concentrations of alpha synuclein at sites of inflammation, with high levels of alpha-synuclein, as compared to a control or healthy subject, correlating with patients appropriate for treatment with a method of the invention.

Based on the data detailed in Example 8, it is believed that administration of an aminosterol reduces the formation of neurotoxic αS aggregates in vivo, and stimulates gastrointestinal motility in patients with neurodiseases such as PD and constipation. The observation that the dose required to achieve a prokinetic response increases with constipation severity supports the hypothesis that the greater the burden of αS impeding neuronal function, the higher the dose of aminosterol required to restore normal bowel function as well as address other symptoms of alpha-synuclein aggregation. The data detailed in Example 8 is the first proof of concept demonstration that directly targeting αS pharmacologically can achieve beneficial GI, autonomic and CNS responses.

This data in Example 8 supports the hypothesis that gastrointestinal dysmotility in neurodiseases such as PD results from the progressive accumulation of αS in the ENS, and that aminosterols can restore neuronal function by displacing αS and stimulating enteric neurons. Improvements were also seen in cognitive function (MMSE scores), hallucinations, REM-behavior disorder (RBD) and sleep. These improvements are unlikely to be due to improved gastric motility and increased absorption of dopaminergic medications, since improvement persisted during the 2-week wash-out period, i.e., in the absence of study drug, thus indicating the likely improvement based upon aminosterol treatment restoring neuronal function by displacing αS and stimulating enteric neurons. These results demonstrate that the ENS in neurodisease such as PD is not irreversibly damaged and can be restored to normal function using the methods of the invention. G. Neurological Disorders or Diseases

In another aspect of the invention, encompassed are methods of treating neurological disease or conditions, or a related symptom, comprising administering to a subject in need a therapeutically effective amount of at least one deuterated aminosterol or a pharmaceutically acceptable salt thereof.

The methods and deuterated aminosterol compositions of the invention can be used to treat and/or prevent neurodiseases such as Alzheimer's disease (AD), Huntington's Disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, and autism.

### 1. Parkinson's Disease

In another aspect of the invention, encompassed are methods of treating PD or a related symptom comprising administering to a subject in need a therapeutically effective amount of at least one deuterated aminosterol or a pharmaceutically acceptable salt thereof.

Parkinson's disease (PD) is a progressive neurodegenerative disorder caused by accumulation of the protein α-synuclein (αS) within the enteric nervous system (ENS), autonomic nerves and brain (Braak et al. 2003). While motor symptoms are still required for a diagnosis of Parkinson's disease (Hughes et al. 1992), non-motor symptoms represent a greater therapeutic challenge (Zahodne et al. 2012). These symptoms include constipation (Ondo et al. 2012; Lin et al. 2014), disturbances in sleep architecture (Ondo et al. 2001; Gjerstad et al. 2006), cognitive dysfunction (Auyeung et al. 2012), hallucinations (Friedman et al. 2016; Diederich et al. 2009), REM behavior disorder (RBD) and depression (Aarsland et al. 2007), all of which result from impaired function of neural pathways not restored by replacement of dopamine. In fact, long-term institutionalization, caregiver burden and decrease in life expectancy correlate more significantly with the severity of these symptoms than with motor symptoms (Goetz et al. 1995).

Parkinson's Disease (PD) is the second most common age-related neurodegenerative disease after AD. PD affects over 1% of the population over the age of 60, which in the US equates to over 500,000 individuals, while in individuals over the age of 85 this prevalence reaches 5%, highlighting the impact that advancing age has on the risk of developing this condition.

Parkinson's disease (PD) is a progressive neurodegenerative disease associated with the accumulation of the protein α-synuclein within the peripheral and central nervous system (CNS). Whilst diagnosis of PD is primarily based on the presence of a combination of motor symptoms, non-motor symptoms, including neuropathic constipation, present a common important therapeutic challenge. In 2003, Braak proposed that PD begins within the GI tract caused when neurotoxic aggregates of α-synuclein form within the ENS, evidenced clinically by the appearance of constipation in a majority of people with PD many years before the onset of motor symptoms. A recent study in rats has demonstrated movement of aggregates of α-synuclein from the ENS to the CNS via the vagus and other afferent nerves. Neurotoxic aggregates accumulated progressively within the brainstem and then dispersed rostrally to structures within the diencephalon, eventually reaching the cerebral hemispheres.

Parkinson's disease (PD) is divided into three stages: preclinical (in which neurodegenerative process is started without evident symptoms or signs); prodromal (in which symptoms and signs are present but insufficient to define a full clinical PD diagnosis); and clinical (in which the diagnosis is achieved based on the presence of classical motor signs).

The so-called gold standard for PD diagnosis entails expert diagnosis based on patient symptoms. PD and prodromal PD diagnosis is probabilistic, made on the basis of the presence of particular motor and non-motor symptoms, physiological pathologies, genetic characteristics, and environmental factors. Diagnosis may include a combination of markers (any disease indicator, whether a symptom, sign, or biomarker) ranging from mild motor symptoms [i.e., UPDRS-1987 version score ≥3 excluding action tremor; or MDS-UPDRS score >6 excluding postural-action tremor; slowness, loss of muscle movements, tremor, rigidity, imbalance, abnormal posture], non-motor symptoms (i.e., REM SBD, olfactory dysfunction, constipation, excessive daytime somnolence, symptomatic hypotension, erectile/urinary dysfunction, depression, cognition), and ancillary diagnostic tests (i.e., abnormal tracer uptake of the presynaptic dopaminergic system: SPECT or positron emission tomography).

Longitudinal studies show reasonable specificity and sensitivity in applying Movement Disorders Society criteria (i.e., research criteria and probability methodology defined by Berg et al., further defining “probable prodromal PD” as 80% certainty) (Berg et al. 2015) for prodromal PD (55% sensitivity; 99% specificity). Indicative signs in these studies include non-motor (in approximate order: REM SBD, hyposmia, constipation, depression, anxiety, executive dysfunction, fatigue, orthostatic hypertension, urinary dysfunction, apathy, pain, sleep problems, dementia, psychosis) and motor (in approximate order: early motor impairments, bradykinesia, tremor, rigidity, fluctuations, freezing, dyskinesias, falls, postural instability, dysphagia) signs that appear at various progressive stages of prodromal and clinical PD.

Parkinson's disease is defined as a synucleinopathy, and synuclein deposition remains the main final arbiter of diagnosis. Additionally, patients with dementia and Lewy bodies are considered as having PD if they meet clinical disease criteria. Imaging (e.g., MRI, single photon emission computed tomography [SPECT], and positron emission tomography [PET]) allows in vivo brain imaging of structural, functional, and molecular changes in PD patients.

There has been research in the last few years identifying particular markers or combinations of markers that are used for probabilistic estimates of prodromal PD. Researchers have identified a timeline of symptoms indicative of prodromal PD and predictive of PD. The presence of each contributes to an estimate of the likelihood of prodromal PD. Some have been adopted for identification of prodromal PD. Other studies use a combination of symptoms and imaging (e.g., hyposmia combined with dopamine receptor imaging has been found to have a high predictive value). In another example, REM sleep behavior disorder (SBD), constipation, and hyposmia were found to be individually common but to rarely co-occur in individuals without PD, leading to a high predictive value for PD.

PD may also be assessed using the Unified Parkinson's Disease Rating Scale (UPDRS) which consists of 42 items in four subscales: (1) Part I, Non-Motor Aspects of Experiences of Daily Living (nM-EDL): cognitive impairment (section 1.1), hallucinations and psychosis (section 1.2), depressed mood (section 1.3), anxious mood (section 1.4), apathy (section 1.5), features of dopamine dysregulation syndrome (section 1.6), sleep problems (section 1.7), daytime sleepiness (section 1.8), pain and other sensations (section 1.9), urinary problems (section 1.10), constipation problems (section 1.11), light headedness on standing (section 1.12), and fatigue (section 1.13); (2) Part II, Motor Aspects of Experiences of Daily Living (M-EDL): speech (section 2.1), saliva & drooling (section 2.2), chewing and swallowing (section 2.3), eating tasks (section 2.4), dressing (section 2.5), hygiene (section 2.6), handwriting (section 2.7), doing hobbies and other activities (section 2.8), turning in bed (section 2.9), tremor (section 2.10), getting out of bed, a car, or a deep chair (section 2.11), walking and balance (section 2.12), and freezing (section 2.13); Part III, Motor Examination: speech (section 3.1), facial expression (section 3.2), rigidity (section 3.3), finger tapping (section 3.4), hand movements (section 3.5), pronation-supination movements of hands (section 3.6), toe tapping (section 3.7), leg agility (section 3.8), arising from chair (section 3.9), gait (3.10), freezing of gait (section 3.11), postural stability (section 3.12), posture (section 3.13), global spontaneity of movement (body bradykinesia) (section 3.14), postural tremor of the hands (section 3.15), kinetic tremor of the hands (section 3.16), rest tremor amplitude (section 3.17), and constancy of rest tremor (section 3.18); Part IV, Motor Complications: time spent with dyskinesias (section 4.1), functional impact of dyskinesias (section 4.2), time spent in the off state (section 4.3), functional impact of fluctuations (section 4.4), complexity of motor fluctuations (section 4.5), and painful off-state dystonia (section 4.6).

Further, symptom-based endpoints can be assessed using known scales. For example, (1) depression can be assessed using the Beck Depression Inventory (BDI-II) (Steer et al. 2000), cognition can be assessed using the Mini Mental State Examination (MMSE) (Palsteia et al. 2018), sleep and REM-behavior disorder (RBD) can be assessed using a daily diary and an RBD questionnaire (RBDQ) (Stiasny-Kolster et al. 2007), and hallucinations can be assessed using the PD hallucinations questionnaire (PDHQ) (Papapetropoulos et al. 2008) and direct questioning. Circadian system status can also be assessed by continuously monitoring wrist skin temperature (Thermochron iButton DS 1921H; Maxim, Dallas) following published procedures (Sarabia et al. 2008).

In another embodiment, administration of a therapeutically effective fixed dose of an aminosterol composition to a PD patient results in improvement of one or more symptoms of Parkinson's disease or on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

PD progression and treatment is particularly difficult in view of patients' development of resistance to dopamine and subsequent dose escalation until no response can be elicited. As explained in Example 8, the data disclosed herein relates to non-dopamine related symptoms. Thus, not to be bound by theory, it is believed that prior or co-administration of an aminosterol composition according to the invention may reduce the dopamine dosage required to elicit a therapeutic effect for Parkinson's symptoms and/or increase the period during which the patient is sensitive to dopamine. It is also theorized that prior or co-administration of an aminosterol composition according to the invention may delay the time period when a patient is advised to begin dopamine therapy. This is significant, as currently patients are encouraged to delay initiation of dopamine treatment as long as possible, as after a period of time subjects become resistant to dopamine.

Data described in Example 8 shows remarkable improvement in a wide variety of symptoms correlated with PD, including a significant and positive effect on bowel function and neurologic symptoms of PD. The study is the first proof of concept demonstration that directly targeting tS pharmacologically can achieve beneficial GI, autonomic and CNS responses in neurodiseases such as PD.

For example, regarding the effect on bowel function, in Stage 1 (single dose), cumulative response rate increased in a dose-dependent fashion from 25% at 25 mg to a maximum of 80% at 200 mg (FIG. 1A). In Stage 2 (daily dosing), the response rate increased in a dose-dependent fashion from 26% at 75 mg to 85.3% at 250 mg (FIG. 1A). The dose required for a bowel response was patient-specific and varied from 75 mg to 250 mg. Median efficacious dose was 100 mg. Average CSBM/week increased from 1.2 at baseline to 3.8 at fixed dose and SBM increased from 2.6 at baseline to 4.5 at fixed dose. Use of rescue medication decreased from 1.8/week at baseline to 0.3 at fixed dose. Consistency based on the Bristol stool scale also improved, increasing from mean 2.7 to 4.1 and ease of passage increased from 3.2 to 3.7. Subjective indices of wellbeing (PAC-QOL) and constipation symptoms (PAC-SYM) also improved during treatment. While the improvement in most stool-related indices did not persist beyond the treatment period, CSBM frequency remained significantly above baseline value.

CNS Symptoms:

Example 8 also describes an analysis with respect to the sleep data, the body temperature data, mood, fatigue, hallucinations, cognition and other motor and non-motor symptoms of PD. CNS symptoms were evaluated at baseline and at the end of the fixed dose period and the wash-out period (Table 11). Moreover, unlike stool-related indices, the improvement in many CNS symptoms persisted during wash-out. The results of treatment were dramatic:

(1) Total UPDRS score was 64.4 at baseline, 60.6 at the end of the fixed dose period and 55.7 at the end of the wash-out period; similarly, the motor component of the UPDRS improved from 35.3 at baseline to 33.3 at the end of fixed dose to 30.2 at the end of wash-out. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 11).

(2) MMSE (cognitive ability) improved from 28.4 at baseline to 28.7 during treatment and to 29.3 during wash-out.

(3) BDI-II (depression) decreased from 10.9 at baseline to 9.9 during treatment and 8.7 at wash-out.

(4) PDHQ (hallucinations) improved from 1.3 at baseline to 1.8 during treatment and 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg.

(5) Improvements were seen in REM-behavior disorder (RBD) and sleep. RBD and total sleep time also improved progressively in a dose-dependent manner. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4).

The data detailed in Example 8 is consistent with the hypothesis that gastrointestinal dysmotility in PD results from the progressive accumulation of αS in the ENS, and that aminosterols can restore neuronal function by displacing αS and stimulating enteric neurons. These results demonstrate that the ENS in PD is not irreversibly damaged and can be restored to normal function.

### 2. Alzheimer's Disease

In another aspect of the invention, encompassed are methods of treating Alzheimer's disease (AD) or a related symptom comprising administering to a subject in need a therapeutically effective amount of at least one deuterated aminosterol or a pharmaceutically acceptable salt thereof.

Alzheimer's disease (AD) is a chronic neurodegenerative disease that usually starts slowly and worsens over time. It is the cause of 60-70% of cases of dementia. As the disease advances, symptoms can include problems with language, disorientation, mood swings, loss of motivation, not managing self care, and behavioral issues. As a person's condition declines, they often withdraw from family and society. Gradually, bodily functions are lost, ultimately leading to death. Although the speed of progression can vary, the typical life expectancy following diagnosis is 3 to 9 years. In 2015, there were approximately 29.8 million people worldwide with AD. It most often begins in people over 65 years of age, although 4% to 5% of cases are early-onset Alzheimer's. It affects about 6% of people 65 years and older. In 2015, dementia resulted in about 1.9 million deaths.

The World Health Organization looked at 23 low- to middle-income nations and estimated that their combined loss in economic output between 2006 and 2015 due to age-related diseases was USD84 billion, and the global cost of AD alone in 2010 was estimated at USD604 billion. Wimo et al. 2013.

The symptoms of AD are primarily marked by cognitive deficits including memory impairment, language dysfunction, and visuospatial skills; functional impairment that may span occupational and social issues (e.g., activities of daily living); and behavioral symptoms including depression, anxiety, aggression and psychosis may also appear as the disease progresses in severity.

At this time, unambiguous diagnosis of AD requires clinical findings of cognitive deficits consistent with AD and post-mortem identification of brain pathologies consistent with AD. The term AD dementia is used to describe dementia that is due to the pathophysiologies of AD. The term “probable Alzheimer's disease” is used in life when a subject demonstrates clinical characteristics of AD and when other possible biological causes of dementia (e.g. PD or stroke) are excluded.

There are currently a variety of art-accepted methods for diagnosing probable AD. Typically, these methods are used in combination. These methods include determining an individual's ability to carry out daily activities and identifying changes in behavior and personality. Dementia of the AD type is also typically characterized by an amnestic presentation (memory deficit) or language, visuospatial or executive function deficits. Cognitive ability/impairment may be determined by art-accepted methods, including, but not limited to, validated instruments that assess global cognition (e.g., the Modified Mini Mental State Examination (3MS-E)), and specific domains such as visual and verbal memory (e.g., the Brief Visuospatial Memory Test (Revised) (BVMT-R) and the Hopkins Verbal Learning Test (Revised) (HVLT-R), respectively), language (e.g., the Generative Verbal Fluency Test (GVFT)) and executive function and attention (e.g., the Digit Span Test (DST)). Dementia due to AD is also defined by insidious onset and a history of worsening cognitive performance.

The criteria for ‘probable Alzheimer's disease’ are described a National Institute of Aging-Alzheimer's Association workgroup (McKhann et al. 2011 Alzheimers Dement, 7: 263-269). According to this workgroup, for people who first exhibit the core clinical characteristics of Alzheimer's disease dementia, evidence of biomarkers associated with the disease may enhance the certainty of the diagnosis.

In another embodiment, administration of a therapeutically effective amount of a deuterated aminosterol composition to an AD subject results in improvement of one or more symptoms of AD on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

### 3. Multiple System Atrophy

In another aspect of the invention, encompassed are methods of treating multiple system atrophy (MSA) or a related symptom comprising administering to a subject in need a therapeutically effective amount of at least one deuterated aminosterol or a pharmaceutically acceptable salt thereof.

MSA is a progressive neurodegenerative disorder characterized by a combination of symptoms that affect both the autonomic nervous system (the part of the nervous system that controls involuntary action such as blood pressure or digestion) and movement. MSA, also known as Shy-Drager syndrome, is a neurodegenerative disorder characterized by tremors, slow movement, muscle rigidity, and postural instability (collectively known as parkinsonism) due to dysfunction of the autonomic nervous system, and ataxia. This is caused by progressive degeneration of neurons in several parts of the brain including the substantia nigra, striatum, inferior olivary nucleus, and cerebellum. There is no known cure for MSA and management is primarily supportive.

Progression of neurodegeneration can be measured using well known techniques. For example, an electroencephalogram (EEG) can be used as a biomarker for the presence and progression of a neurodegenerative disease. S. Morairty, 2013. Another exemplary technique that can be used to measure progression of neurodegeneration of MRI. Rocca et al. 2017.

A variety of neuroimaging techniques may be useful for the early diagnosis and/or measurement of progression of MSA. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, 2012. Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles.

In another embodiment, administration of a therapeutically effective fixed dose of an aminosterol composition to an MSA patient results in improvement of one or more symptoms of MSA, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool or assessment. 4. Schizophrenia

In another aspect of the invention, encompassed are methods of treating schizophrenia or a related symptom comprising administering to a subject in need a therapeutically effective amount of at least one deuterated aminosterol or a pharmaceutically acceptable salt thereof.

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease.

The mainstay of treatment is antipsychotic medication, along with counselling, job training and social rehabilitation. However, the 2013 study found that in general, the higher the anti-psychotic medication doses, the greater the loss of brain tissue.

About 0.3-0.7% of people are affected by schizophrenia during their lifetimes. In 2013 there were an estimated 23.6 million cases globally. Males are more often affected, and on average experience more severe symptoms. About 20% of people do well and a few recover completely. About 50% have lifelong impairment. Social problems, such as long-term unemployment, poverty and homelessness are common. The average life expectancy of people with the disorder is ten to twenty-five years less than for the general population. This is the result of increased physical health problems and a higher suicide rate (about 5%). In 2015 an estimated 17,000 people worldwide died from behavior related to, or caused by, schizophrenia.

While not wished to be bound by theory, it is theorized that administration of a therapeutically effective amount of a deuterated aminosterol composition to a schizophrenia patient may treat and/or prevent schizophrenia or any one or more symptoms thereof. In some embodiments, the administration may be oral—resulting in absorption in the ENS. In some embodiments, the administration may be intranasal—resulting in stimulation of neurogenesis, which has a positive impact on the loss of brain tissue characteristic of schizophrenia subjects.

In one embodiment of the invention, administration of a therapeutically effective fixed dose of an aminosterol composition to a schizophrenia patient results in improvement of one or more symptoms as determined by a clinically recognized psychiatric symptom rating scale. Examples of such rating scales include for example, the Positive and Negative Syndrome Scale (PANSS), the Psychotic Symptom Rating Scales (PSYRATS), the Quality of Life Scale (QLS), the Schizophrenia Cognition Rating Scale (SCoRS), the Drug Attitude Inventory (DAI), and the Abnormal Involuntary Movement Scale (AIMS).

In another embodiment, administration of a therapeutically effective amount of a deuterated aminosterol composition to a schizophrenia patient results in improvement of one or more symptoms as determined by a clinically recognized psychiatric symptom rating scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Improvement can be measured using any clinically recognized tool or assessment.

### 5. Autism

In another aspect of the invention, encompassed are methods of treating autism or a related symptom comprising administering to a subject in need a therapeutically effective amount of at least one deuterated aminosterol or a pharmaceutically acceptable salt thereof.

Autism, or autism spectrum disorder, refers to a range of conditions characterized by challenges with social skills, repetitive behaviors, speech and nonverbal communication, as well as by unique strengths and differences. There are many types of autism, caused by different combinations of genetic and environmental influences.

Autism's most-obvious signs tend to appear between 2 and 3 years of age. In some cases, it can be diagnosed as early as 18 months. Some developmental delays associated with autism can be identified and addressed even earlier.

The Centers for Disease Control and Prevention (CDC) estimates autism's prevalence as 1 in 59 children in the United States. This includes 1 in 37 boys and 1 in 151 girls. Around one third of people with autism remain nonverbal. Around one third of people with autism have an intellectual disability. Certain medical and mental health issues frequently accompany autism. They include gastrointestinal (GI) disorders, seizures, sleep disturbances, attention deficit and hyperactivity disorder (ADHD), anxiety and phobias.

Experts are still uncertain about of all the causes of autism. In all likelihood, there are multiple causes. It appears that a number of different circumstances, including environmental, biologic, and genetic factors, set the stage for autism and make a child more likely to have the disorder. It is likely that genetics play a large factor in the development of autism. Identical twins are more likely to both be affected than twins who are fraternal (not genetically identical). In a family with one autistic child, the chance of having another child with autism is about 5 percent—or one in 20—which is much higher than in the normal population. Research also has found that some emotional disorders (such as manic depression) occur more often in families of a child with autism.

At least one group of researchers has found a link between an abnormal gene and autism. The gene may be just one of three to five or more genes that interact in some way to cause the condition. Scientists suspect that a faulty gene or genes might make a person more likely to develop autism when there are also other factors present, such as a chemical imbalance, viruses or chemicals, or a lack of oxygen at birth.

Other potential causes of autism are environmental toxins, including pesticides and heavy metals such as mercury. Heavy metals are certainly more commonly encountered in the environment now than they were in the past. It may be that people with autism or those at higher risk for developing it are more sensitive than others to these toxins.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. But by late adolescence, pruning eliminates about half of these cortical synapses. In addition, many genes linked to autism are known to affect the development or function of brain synapses. The study also found that the brain cells from individuals with autism were filled with damaged parts and deficient in signs of a normal breakdown pathway called “autophagy.” Tang et al. 2014.

Thus, one embodiment of the invention is directed to methods of treating autism comprising administering a therapeutically effective fixed dose of an aminosterol composition according to the invention. In one embodiment, treatment results in improvement in one or more characteristics of autism. Such characteristics can be, for example, communication skills, social interaction, sensory sensitivity, and behavior. Improvement can be measured using any clinically recognized tool or assessment.

For example, the methods of the invention may show an improvement in one or more characteristics of autism, such as behavior, communication, mood, etc., as measured by a medically recognized scale. The improvement may be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

### 6. Other Neurodiseases

The methods and deuterated aminosterol compositions of the invention may also be useful in treating and/or preventing a variety of other neurodiseases and/or conditions. Examples of such neurodiseases and conditions include, but are not limited to, Huntington's disease, Progressive supranuclear palsy, Frontotemporal dementia (FTD), vascular dementia, Amyotrophic lateral sclerosis (ALS), multiple sclerosis (MS), spinal muscular atrophy (SMA), Friedreich's ataxia, acute traumatic injury to the central nervous system, including the spinal cord, stroke, acute head and/or spine injury, degenerative processes associated with aging, including memory loss (“dementia of aging”), cerebral palsy, epilepsy, and peripheral sensory neuropathy.

In another aspect of the invention, encompassed are methods of treating Huntington's disease or a related symptom comprising administering to a subject in need a therapeutically effective amount of at least one deuterated aminosterol or a pharmaceutically acceptable salt thereof.

Huntington's disease (HD) is a fatal genetic disorder that causes the progressive breakdown of nerve cells in the brain. It deteriorates a person's physical and mental abilities during their prime working years and has no cure. Full-time care is required in the later stages of the disease. Symptoms of Huntington's disease most commonly become noticeable between the ages of 35 and 44 years, but they can begin at any age from infancy to old age. The most characteristic initial physical symptoms are jerky, random, and uncontrollable movements called chorea. Suicide is the cause of death in about 9% of cases. Death typically occurs 15 to 20 years from when the disease was first detected.

In another aspect of the invention, encompassed are methods of treating Progressive supranuclear palsy or a related symptom comprising administering to a subject in need a therapeutically effective amount of at least one deuterated aminosterol or a pharmaceutically acceptable salt thereof.

Progressive supranuclear palsy, also called Steele-Richardson-Olszewski syndrome, is an brain disorder that causes serious problems with walking, balance and eye movements. The disorder results from deterioration of cells in areas of the brain that control body movement and thinking. There is no known cure for PSP and management is primarily supportive.

In another aspect of the invention, encompassed are methods of treating Frontotemporal dementia (FTD) or a related symptom comprising administering to a subject in need a therapeutically effective amount of at least one deuterated aminosterol or a pharmaceutically acceptable salt thereof.

Frontotemporal dementia (FTD) is a group of related conditions resulting from the progressive degeneration of the temporal and frontal lobes of the brain. These areas of the brain play a significant role in decision-making, behavioral control, emotion and language. The frontotemporal dementias (FTD) encompass six types of dementia involving the frontal or temporal lobes. They are: behavioral variant of FTD, semantic variant primary progressive aphasia, nonfluent agrammatic variant primary progressive aphasia, corticobasal syndrome, progressive supranuclear palsy, and FTD associated with motor neuron disease. Currently, there is no cure for FTD.

In another aspect of the invention, encompassed are methods of treating vascular dementia or a related symptom comprising administering to a subject in need a therapeutically effective amount of at least one deuterated aminosterol or a pharmaceutically acceptable salt thereof.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes.

In another aspect of the invention, encompassed are methods of treating Amyotrophic lateral sclerosis (ALS) or a related symptom comprising administering to a subject in need a therapeutically effective amount of at least one deuterated aminosterol or a pharmaceutically acceptable salt thereof.

Amyotrophic lateral sclerosis (ALS), also known as motor neurone disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

In another aspect of the invention, encompassed are methods of treating Multiple sclerosis (MS) or a related symptom comprising administering to a subject in need a therapeutically effective amount of at least one deuterated aminosterol or a pharmaceutically acceptable salt thereof.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. While the cause is not clear, the underlying mechanism is thought to be either destruction by the immune system or failure of the myelin-producing cells. Proposed causes for this include genetics and environmental factors such as being triggered by a viral infection. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

In another aspect of the invention, encompassed are methods of treating spinal muscular atrophy (SMA) or a related symptom comprising administering to a subject in need a therapeutically effective amount of at least one deuterated aminosterol or a pharmaceutically acceptable salt thereof.

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. SMA is the most common genetic cause of infant death. In December 2016, nusinersen became the first approved drug to treat SMA while several other compounds remain in clinical trials.

In another aspect of the invention, encompassed are methods of treating Friedreich's ataxia or a related symptom comprising administering to a subject in need a therapeutically effective amount of at least one deuterated aminosterol or a pharmaceutically acceptable salt thereof.

Friedreich's ataxia is an autosomal recessive inherited disease that causes progressive damage to the nervous system. It manifests in initial symptoms of poor coordination such as gait disturbance; it can also lead to scoliosis, heart disease and diabetes, but does not affect cognitive function. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses).

Progression of neurodegeneration can be measured using well known techniques. For example, an electroencephalogram (EEG) can be used as a biomarker for the presence and progression of a neurodegenerative disease. S. Morairty, “Detecting Neurodegenerative Diseases Before Damage Is Done,” SRI International (Jul. 26, 2013) (https://www.sri.com/blog/detecting-neurodegenerative-diseases). Another exemplary technique that can be used to measure progression of neurodegeneration of MRI. Rocca et al., “The Role of T1-Weighted Derived Measures of Neurodegeneration for Assessing Disability Progression in Multiple Sclerosis,” Front Neurol., 8:433 (Sep. 4, 2017).

A variety of neuroimaging techniques may be useful for the early diagnosis and/or measurement of progression of neurodegenerative disorders. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, “Neuroimaging in the early diagnosis of neurodegenerative disease,” Transl. Neurodegener., 1: 5 (2012). Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. Id.

In one embodiment of the invention, the progression or onset of a neurodegenerative disorder is slowed or prevented over a defined time period, following administration of a therapeutically effective amount of a deuterated aminosterol or a pharmaceutically acceptable salt thereof according to the invention to a subject in need, as measured by a medically-recognized technique. For example, the progression or onset of a neurodegenerative disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The period of time over which the progression or onset of a neurodegenerative disorder is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the invention, a neurodegenerative disorder may be positively impacted by administration of a deuterated aminosterol or a pharmaceutically acceptable salt thereof according to the invention. A “positive impact” includes for example slowing advancement of the condition, improving one or more symptoms, etc.

### H. Microbial Infections

The methods and deuterated aminosterol compositions of the invention may also be useful in treating and/or preventing a variety of other microbial infections. Thus, in some embodiments, the subject in need has a condition selected from the group consisting of viral infections, antimicrobial infections, Gram-negative and Gram-positive bacterial infections, Mycobacteria infections, fungal infections, and protozoan infections.

As used herein the term “microorganism” refers to microscopic organisms and taxonomically related macroscopic organisms within the categories of algae, bacteria, fungi (including lichens), protozoa, viruses, and subviral agents. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism. As used herein the term “pathogen,” and grammatical equivalents, refers to an organism, including microorganisms, that causes disease in another organism (e.g., animals and plants) by directly infecting the other organism, or by producing agents that causes disease in another organism (e.g., bacteria that produce pathogenic toxins and the like).

In one embodiment of the present technology, the condition to be treated is a chronic disease suspected to be of viral origin. For example, the condition to be treated can be multiple sclerosis, Type I diabetes, Type II diabetes, atherosclerosis, cardiomyopathies, Kawaski disease, aplastic anemia, etc.

In contrast to traditional antiviral therapies, viruses are not expected to develop resistance to the deuterated aminosterol. This is because unlike conventional antiviral therapies, the deuterated aminosterol does not act upon a single mechanism by which a virus infects a cell. Rather, the deuterated aminosterol changes the cell structure for a period of time during which the virus cannot infect the cell. In contrast, certain anti-HIV drugs target the CD4 receptor and other antiviral drugs target inhibition of replication. Viral variants can circumvent each of these targeted antiviral therapies.

In one embodiment of the present technology, the deuterated amino sterol does not demonstrate an altered IC₅₀ or IC₉₀ (drug concentration required to inhibit viral growth by 50% or 90% respectively) over time. In other embodiments of the present technology, the deuterated aminosterol demonstrates an IC₅₀ or IC₉₀ which does not increase by more than 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, or 30% over time. In other embodiments of the present technology, the time period over which the change in IC₅₀ or IC₉₀ (or lack thereof) is measured is 1 week, 2 weeks, 3 weeks, 4 weeks, 1 month, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1 year, 1.5 years, 2 years, 2.5 years, 3 years, 3.5 years, 4 years, 4.5 years, or 5 years.

The present technology is directed to methods of treating and/or preventing microbial infections, and in particular pathogenic microorganisms, comprising administering a therapeutically effective amount of an aminosterol composition described herein to a subject in need. As noted above, this method encompasses using an aminosterol composition described herein in combination with conventional antimicrobial treatments to treat and/or prevent infections. 1. Bacterial Infection

The bacterial infection to be treated and/or prevented can be due to a gram negative bacteria, gram positive bacteria, Mycobacteria, bacterial spore, or any combination thereof. Pathogenic bacteria are a major cause of human death and disease and cause infections such as tetanus, typhoid fever, diphtheria, syphilis, cholera, foodborne illness, leprosy and tuberculosis. Examples of gram positive bacteria include, but are not limited to genera such as Staphylococcus, Streptococcus, Enterococcus, (which are cocci) and Bacillus, Corynebacterium, Nocardia, Clostridium, Actinobacteria, and Listeria. Examples of gram negative bacteria include, but are not limited to, Escherichia coli, Salmonella, Shigella, Enterobacteriaceae, Neisseria, Pseudomonas, Moraxella, Helicobacter, Stenotrophomonas, Bdellovibrio, acetic acid bacteria, Legionella and alpha-proteobacteria as Wolbachia and many others. Other notable groups of Gram-negative bacteria include the cyanobacteria, spirochaetes, green sulfur and green non-sulfur bacteria. Medically relevant Gram-negative bacilli include a multitude of species. Some of them primarily cause respiratory problems (Hemophilus influenzae, Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi). Gram-negative bacteria associated with nosocomial infections include Acinetobacter baumannii, which cause bacteremia, secondary meningitis, and ventilator-associated pneumonia in intensive care units of hospital establishments. Relevant Mycobacteria include M. tuberculosis complex (MTBC), M. tuberculosis, M. bovis, M. africanum, and M. microti; M. leprae, M. avium complex, M. avium paratuberculosis, M. avium sylvaticum, or any of the Mycobacterial species demonstrated to cause disease in man and/or animals.

### 2. Fungal Infections

The fungal, yeast and/or mold infection to be treated, prevented, and/or cured may be a tinea infection, dermatophytoses, or a dermatophytoma. Examples of fungal microorganisms include, but are not limited to, Trichophyton spp., Epidermophyton spp., Fusarium spp., Aspergillus spp., Paecilomyces spp., Acremonium spp., Scytalydium spp., Scopulariopsis spp., Scedosporium spp., Alternaria spp., Epicoccum spp., Curvularia spp., Candida spp., Phoma spp., Chaetomium spp., and Microsporum spp.

Molds include, but are not limited to infections caused by the fungi Acremonium spp., Aspergillus spp. (e.g., A. sydowii, A. terreus, A. niger), Fusarium spp. (e.g., F. oxysporum, F. solani, F. semitectum), Scopulariopsis spp. (e.g., Scopulariopsis brevicaulis), Scedosporuim spp., Alternaria spp., Paecilomyces lilacinus, Epiccocum nigrum, Phoma spp. Chaetomium spp., Curvularia spp., Onychocola canadensis, and Scytalidium spp., (e.g., S. dimidiatum).

Yeast, as defined herein, include, but are not limited to, Candida species causing yeast infections.

### I. Cancer and Neovascularization Diseases and Conditions

The present technology is directed to methods of treating disease states known to be associated with pathological neovascularization, such as cancer, vascular disorders of the eye, including macular degeneration, such as age-related macular degeneration, retinopathy of prematurity, corneal neovascularization, diabetic retinopathy, and disorders of neovascularization. Examples include but are not limited to malignant and cancerous tumors. The methods comprise administering a therapeutically effective amount of a deuterated aminosterol composition described herein to a subject in need.

Examples of tumors that can be treated with the compositions of the present technology include, but are not limited to, breast, brain, lung (e.g., non-small cell lung cancer), and CNS. An example of a solid brain tumor that can be treated with a composition according to the present technology is a malignant glioma. Other examples of cancers that can be treated with compositions according to the present technology include, but are not limited to, prostate cancer.

### J. Patient Populations

The disclosed compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of one or more disorders. For example, genetic markers of Alzheimer's disease (e.g. APOE4) or family history may be used as signs to identify subjects likely to develop Alzheimer's disease. Thus, in some embodiments relating to disorders for which certain genetic or hereditary signs are known, prevention may involve first identifying a patient population based on one of the signs. Alternatively, certain symptoms are considered early signs of particular disorders. For example, constipation is considered an early sign of Parkinson's disease. Thus, in some embodiments relating to Parkinson's disease, a patient population may be selected for being “at risk” for developing Parkinson's disease based on age and experiencing constipation. An exemplary population is young adults between the ages of about 20 and about 40 experiencing constipation characterized by less than 3 bowel movements per week. These patients can be targeted and monitored for prevention of Parkinson's disease onset. Further genetic or hereditary signs may be used to refine the patient population.

### IV. Combination Therapy

The deuterated aminosterol compositions of the present technology may be administered alone or in combination with other therapeutic agents. Any active agent known to be useful in treating a condition or disease described herein can be used in conjunction with the deuterated aminosterol compositions of the present technology. For example, the deuterated aminosterol compositions described herein may be administered in combination with compounds such as chemotherapeutic agents, antibiotics, steroidal and non-steroidal anti-inflammatories, conventional immunotherapeutic agents, antiviral agents, anti-inflammatory agents, active agents used in treating gastrointestinal disorders, active agents used in treating hallucinations, active agents used in treating depression, active agents used in treating neurodiseases, active agents used in treating microbial infections, etc.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

### V. Methods of Preparing Deuterated Forms

In another aspect, provided herein are processes to prepare a deuterated form of squalamine, or a pharmaceutically acceptable salt thereof. In some embodiments, the process is as described in the Example(s) shown below.

In some embodiments, provided herein is a process to prepare D₇-1436, the process comprising:

a) reacting compound (A)

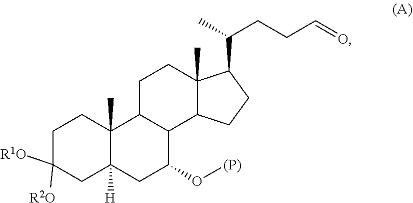

with an organometallic reagent to provide compound (B):

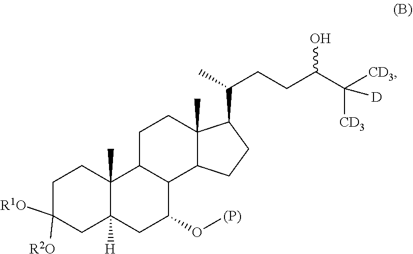

wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether;

b) reacting compound (B) with sulfur trioxide-pyridine complex to provide compound (C)

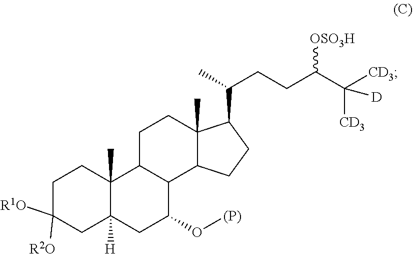

c) reacting compound (C) under acidic conditions to provide compound (D)

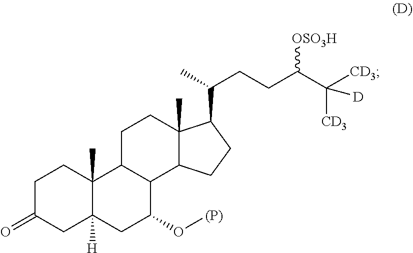

d) reacting compound (D) under reductive amination conditions to provide compound (E)

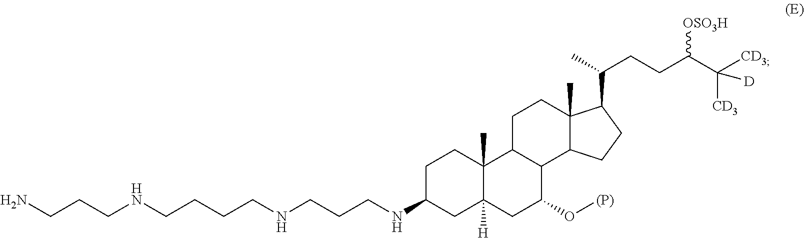

and

e) reacting compound (E) under conditions to remove P to provide D₇-1436

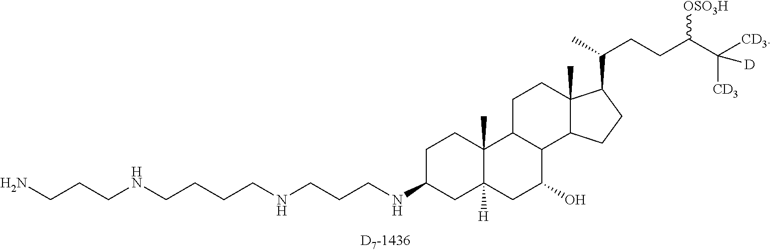

In some embodiments, provided herein is a process to prepare D₇-Squalamine, the process comprising:

a) reacting compound (A)

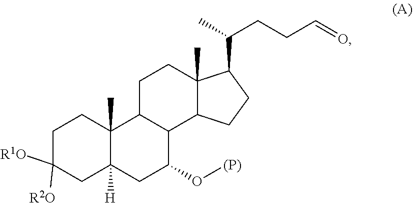

with an organometallic reagent to provide compound (B):

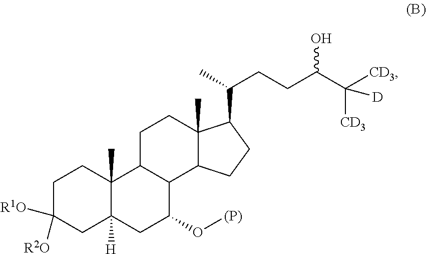

wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether;

b) reacting compound (B) with sulfur trioxide-pyridine complex to provide compound (C)

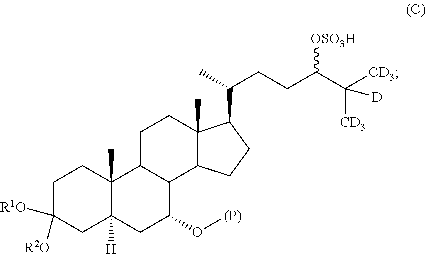

c) reacting compound (C) under acidic conditions to provide compound (D)

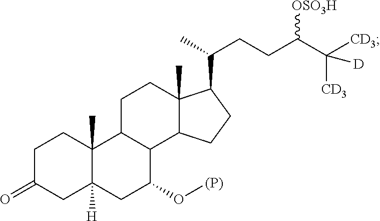

d) reacting compound (D) under reductive amination conditions to provide compound (F)

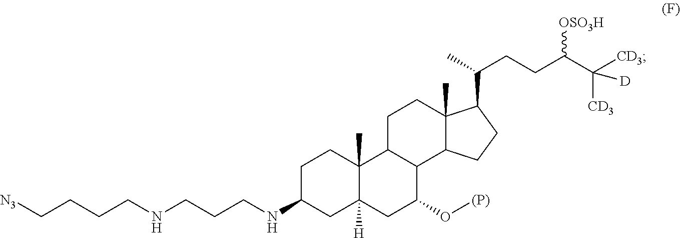

e) reacting compound (F) under reducing conditions to provide compound (G)

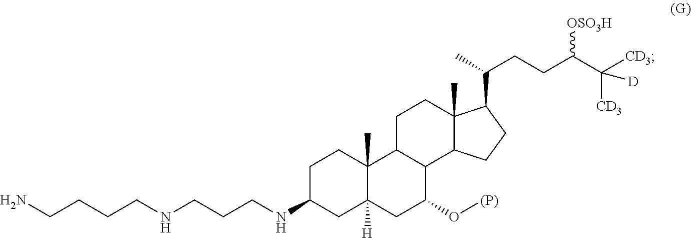

and

f) reacting compound (G) under conditions to remove P to provide D₇-Squalamine

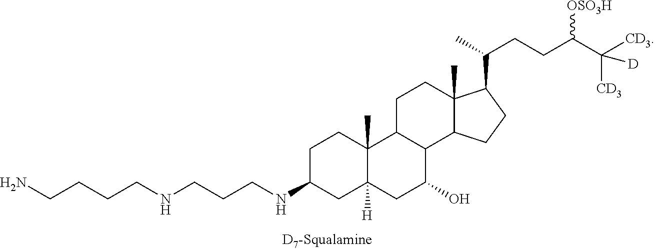

In some embodiments, R¹ and R² of compound (A) form a cyclic ketal. In some embodiments, R¹ and R² of compound (A) form a 1,3-dioxolane.

In some embodiments, P of compound (A) is —C(O)(C₆H₅).

In some embodiments, compound (A) is

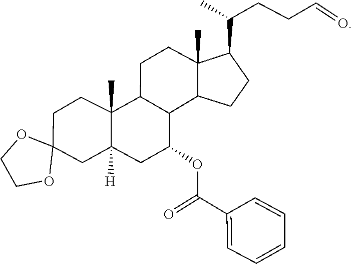 [SMILES: C[C@H](CCC=O)[C@H]1CCC2C3C(CC[C@@]21C)[C@@]1(C)CCC2(C[C@@H]1C[C@H]3OC(=O)c1ccccc1)OCCO2]

In some embodiments, the organometallic compound of step a) is a Grignard reagent. In some embodiments, the organometallic compound of step a) is deuterated diisopropyl zinc reagent; step a) further comprises contacting compound (A) with a chiral ligand; and step a) provides enantio-enriched compound (B). In further embodiments, the ligand is (1S,2R)-2-(dibutylamino)-1-phenylpropan-1-ol.

In some embodiments, the acidic conditions of step c) comprise, consist essentially of, or consist of acidic ion-exchange resin in acetone. In some embodiments, the acidic ion-exchange resin is an ion-exchange resin with sulfonic acid functionality.

In some embodiments, the reductive amination conditions of step d) to provide compound (E) comprise spermine and a reducing agent. In some embodiments, the reducing agent is sodium borohydride.

In some embodiments, the reductive amination conditions of step d) to provide compound (F) comprise azidospermidine and a reducing agent. In some embodiments, the reducing agent is sodium borohydride.

In some embodiments, the reducing conditions to provide compound (G) comprise, consist essentially of, or consist of catalytic hydrogenation conditions. In some embodiments, the catalytic hydrogenation conditions comprise, consist essentially of, or consist of hydrogenation with Raney nickel.

In some embodiments, the conditions to remove P are basic conditions. In some embodiments, the basic conditions comprise basic alcoholic conditions. In some embodiments, the basic alcoholic conditions comprise, consist essentially of, or consist of hydroxide in methanol. In some embodiments, the basic conditions comprise, consist essentially of, or consist of a methanolic potassium hydroxide solution.

In some embodiments, compound (D) is reacted with CBZ-Spermidine diHCl under reductive amination conditions to provide compound (F′)

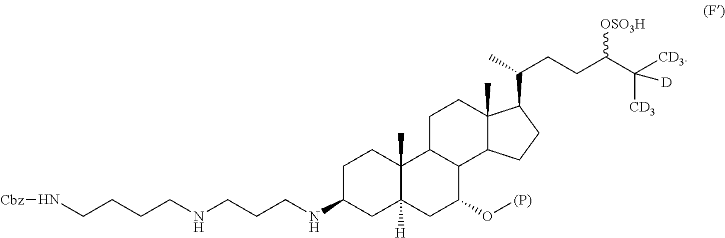

In some embodiments, the reductive amination conditions to provide compound (F′) comprise sodium borohydride.

In some embodiments, compound (F′) is reacted under conditions to remove Cbz to provide compound (G). In some embodiments, the conditions to remove Cbz comprise, consist essentially of, or consist of hydrogenation conditions. In some embodiments, the hydrogenation conditions comprise, consist essentially of, or consist of hydrogenation with a palladium on carbon catalyst.

In some embodiments, provided herein is a compound selected from:

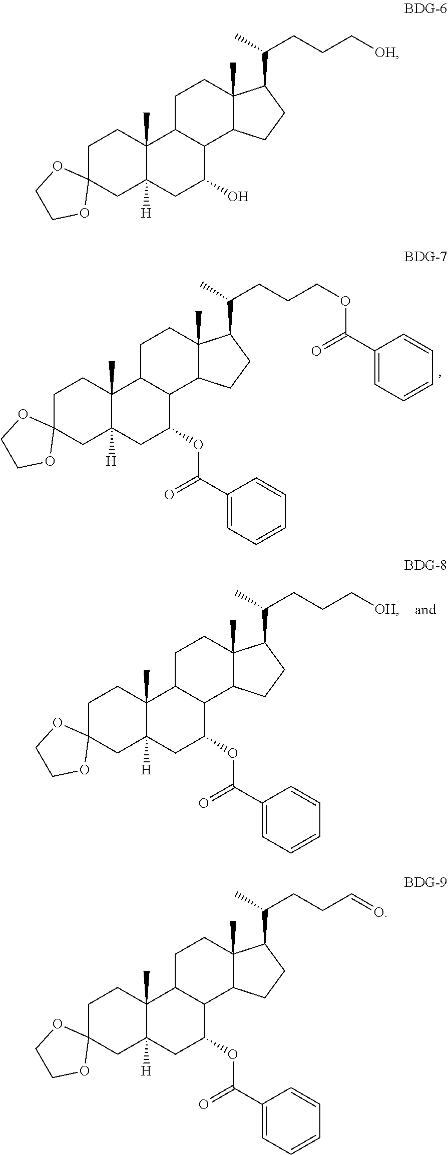

In some embodiments, provided herein is a process to prepare compound (O), the process comprising:

a) reacting compound (H)

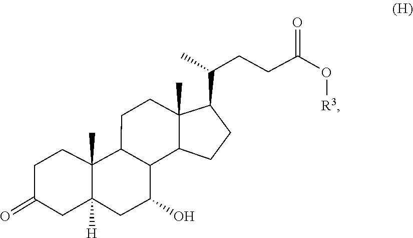

under acidic conditions to provide compound (J)

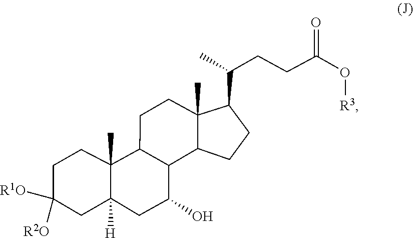

wherein each of R¹ and R² is independently C₁₋₆ alkyl, or R¹ and R², together with the oxygen atoms to which they are attached, form a heterocyclic ring; and R³ is substituted or unsubstituted C₁₋₆ alkyl;

b) reacting compound (J) with a reducing agent to provide compound (K)

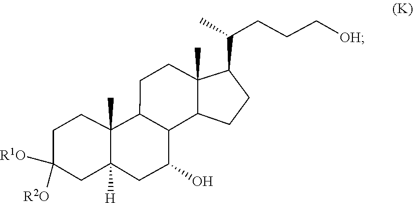

c) reacting compound (K) under conditions to introduce a protecting group P′ to provide compound (L)

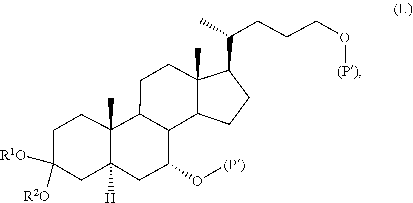

wherein P′ is substituted or unsubstituted C₁₋₆ alkyl ester, substituted or unsubstituted aryl ester, or substituted or unsubstituted heteroaryl ester;

d) reacting compound (L) under conditions to selectively remove one P′ to provide compound (M)

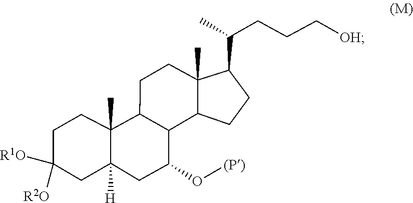

e) reacting compound (M) under oxidation conditions to provide compound (N)

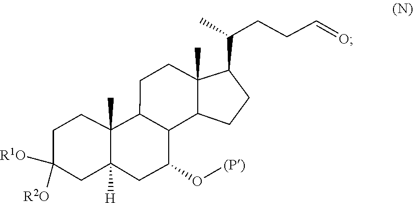

and

f) reacting compound (N) with an organometallic reagent to provide compound (O)

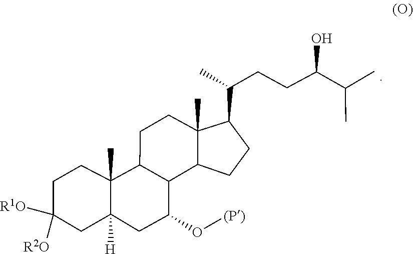

In some embodiments, R³ of compound (H) is unsubstituted C₁₋₆ alkyl. In some embodiments, R³ of compound (H) is methyl. In some embodiments, R³ of compound (H) is ethyl.

In some embodiments, the acidic conditions of step a) to provide compound (J) comprise, consist essentially of, or consist of ethylene glycol, p-toluenesulfonic acid-hydrate, and dichloromethane.

In some embodiments, R¹ and R² of compound (J) form a cyclic ketal. In some embodiments, R¹ and R² of compound (J) form a 1,3-dioxolane.

In some embodiments, the reducing agent of step b) to provide compound (K) is lithium aluminum hydride.

In some embodiments, P′ of compound (L) is unsubstituted aryl ester. In some embodiments, P′ of compound (L) is —C(O)(C₆H₅).

In some embodiments, the conditions to introduce the protecting group P′ to provide compound (L) in step c) comprise, consist essentially of, or consist of benzoyl chloride, 4-dimethylaminopyridine, and dichloromethane.

In some embodiments, the conditions to selective remove one P′ to provide compound (M) in step d) are basic conditions. In some embodiments, the basic conditions comprise basic alcoholic conditions. In some embodiments, the basic alcoholic conditions comprise, consist essentially of, or consist of hydroxide in methanol. In some embodiments, the basic conditions comprise, consist essentially of, or consist of a methanolic potassium hydroxide solution. In some embodiments, the basic conditions comprise, consist essentially of, or consist of potassium hydroxide in THF/methanol.

In some embodiments, the oxidation conditions of step e) to provide compound (N) comprise or consist essentially of TEMPO and bleach under basic conditions. In some embodiments, the oxidation conditions of step e) to provide compound (N) comprise, consist essentially of, or consist of TEMPO, bleach, sodium bicarbonate, potassium bromide, and water.

In some embodiments, the organometallic reagent to provide compound (O) in step f) is diisopropyl zinc reagent. In some embodiments, the organometallic reagent is used in conjunction with a chiral ligand. In still further embodiments, the organometallic reagent is diisopropyl zinc reagent and the chiral ligand is (1S,2R)-2-(dibutylamino)-1-phenylpropan-1-ol.

In some embodiments, provided herein is a process to prepare compound (O), the process comprising:

a) reacting compound (P)

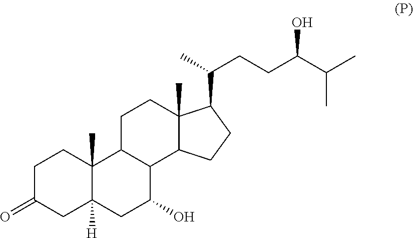 [SMILES: CC(C)[C@H](O)CC[C@@H](C)[C@H]1CCC2C3C(CC[C@@]21C)[C@@]1(C)CCC(=O)C[C@@H]1C[C@H]3O]

under acidic conditions to provide compound (Q):

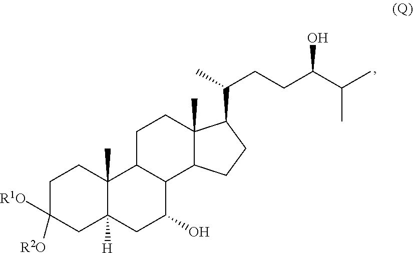

wherein each of R¹ and R² is independently C₁₋₆ alkyl, or R¹ and R², together with the oxygen atoms to which they are attached, form a heterocyclic ring;

b) reacting compound (Q) under conditions to introduce a protecting group P′ to provide compound (R)

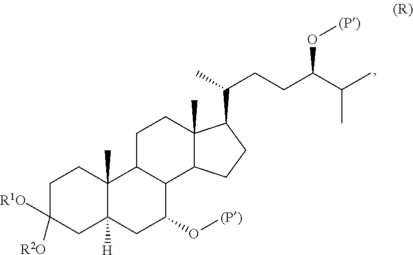

wherein P′ is substituted or unsubstituted C₁₋₆ alkyl ester, substituted or unsubstituted aryl ester, or substituted or unsubstituted heteroaryl ester; and

c) reacting compound (R) under conditions to selectively remove one P′ to provide compound (O)

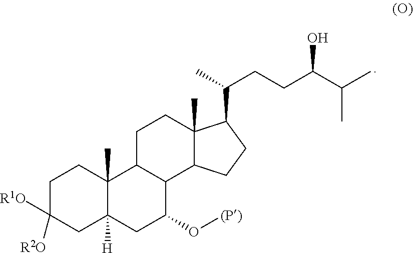

In some embodiments, the acidic conditions to provide compound (Q) in step a) comprise, consist essentially of, or consist of ethylene glycol, oxalic acid, and acetonitrile.

In some embodiments, R¹ and R² of compound (Q) form a cyclic ketal. In some embodiments, R¹ and R² of compound (Q) form a 1,3-dioxolane.

In some embodiments, P′ of compound (R) is unsubstituted aryl ester. In some embodiments, P′ of compound (R) is —C(O)(C₆H5).

In some embodiments, the conditions to introduce the protecting group P′ to provide compound (R) in step b) comprise, consist essentially of, or consist of benzoyl chloride, 4-dimethylaminopyridine, and dichloromethane.

In some embodiments, the conditions to selective remove one P′ to provide compound (O) in step c) are basic conditions. In some embodiments, the basic conditions comprise basic alcoholic conditions. In some embodiments, the basic alcoholic conditions comprise, consist essentially of, or consist of hydroxide in methanol. In some embodiments, the basic conditions comprise, consist essentially of, or consist of a methanolic potassium hydroxide solution. In some embodiments, the basic conditions comprise, consist essentially of, or consist of potassium hydroxide in THF/methanol.

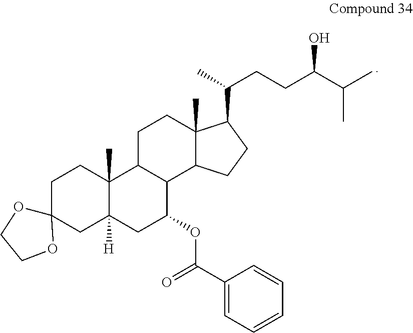 [SMILES: CC(C)[C@H](O)CC[C@@H](C)[C@H]1CCC2C3C(CC[C@@]21C)[C@@]1(C)CCC2(C[C@@H]1C[C@H]3OC(=O)c1ccccc1)OCCO2]

In some embodiments, compound (O) is Compound 34

In some embodiments, provided herein is a process to prepare CBZ-spermidine

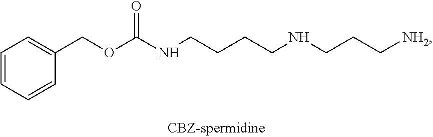 [SMILES: NCCCNCCCCNC(=O)OCc1ccccc1]

or a salt thereof, the process comprising:

a) reacting 4-amino-1-butanol under conditions to introduce a Cbz group to provide compound (S)

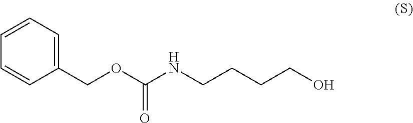 [SMILES: O=C(NCCCCO)OCc1ccccc1]

(b) sulfonylating compound (S) to provide compound (T)

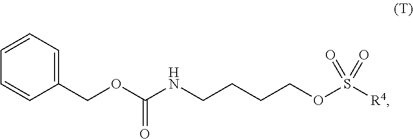

wherein R⁴ is substituted or unsubstituted C₁₋₆ alkyl, or substituted or unsubstituted aryl; and

(c) reacting compound (T) with 1,3-diaminopropane to provide CBZ-spermidine, or the salt thereof.

In some embodiments, the conditions to introduce a Cbz group to provide compound (S) in step a) comprise, consist essentially of, or consist of benzylchloroformate, triethylamine, and dichloromethane.

In some embodiments, R⁴ is substituted or unsubstituted C₁₋₆ alkyl. In some embodiments, R⁴ is substituted or unsubstituted aryl. In some embodiments, R⁴ is unsubstituted C₁₋₆ alkyl. In some embodiments, R⁴ is methyl.

In some embodiments, the sulfonylating step of step b) to provide compound (T) comprises, consists essentially of, or consists of contacting compound (S) with alkyl sulfonyl chloride under basic conditions. In some embodiments, the sulfonylating step of step b) to provide compound (T) comprises, consists essentially of, or consists of contacting compound (S) with methyl sulfonyl chloride and triethylamine in anhydrous solvent. In some embodiments, the anhydrous solvent is methyltetrahydrofuran.

In some embodiments, step c) to provide CBZ-spermidine is performed in an anhydrous solvent. In some embodiments, the anhydrous solvent is methyltetrahydrofuran.

In some embodiments, step c) provides the di-hydrochloride salt of CBZ-spermidine.

### VI. Definitions

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term, for example, ±1%, ±2%, ±3%, ±4%, ±5%, ±6%, ±7%, ±8%, ±9% or ±10%. As used herein in the context of values of degrees 2-theta (20), “about” refers to ±0.20 in some embodiments, or in some embodiments, ±0.10. As used herein in the context of the temperature(s) of endothermic peak(s) within a differential scanning calorimetry thermogram, “about” refers to ±0.4 OC.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “hydrate” means a compound which further includes a stoichiometric or non-stoichiometric amount of water bound by non-covalent intermolecular forces.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The term “substantially” generally refers to at least 90% similarity. In some embodiments, in the context of a first X-ray powder diffraction pattern being substantially as shown in a second X-ray powder diffraction pattern, “substantially” refers to ±0.20. In some embodiments, in the context of a first differential scanning calorimetry thermogram being substantially as shown in a second differential scanning calorimetry thermogram, “substantially” refers to ±0.4° C. In some embodiments, in the context of a first thermogravimetric analysis being substantially as shown in a second thermogravimetric analysis, “substantially” refers to ±0.4% weight. In some embodiments, “substantially purified” refers to at least 95% purity. This includes at least 96, 97, 98, or 99% purity. In further embodiments, “substantially purified” refers to about 95, 96, 97, 98, 99, 99.5, or 99.9% purity, including increments therein.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

In general, organic groups which are “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. The present disclosure is understood to include embodiments where, for instance a “substituted alkyl” optionally contains one or more alkene and/or alkyne. A substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); alkyl; haloalkyl (such as, but not limited to, trifluoroalkyl); hydroxyls; alkoxy, alkenoxy, alkynoxy, aryloxy, aralkyloxy, heterocyclyloxy, and heterocyclylalkoxy groups; aryl groups; heteroaryl groups; cycloalkyl groups; heterocyclyl groups; carbonyls (oxo); carboxyls; esters; urethanes; oximes; carbamates, hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocycle and heteroaryl groups also include rings and fused ring systems in which a bond to a hydrogen atom is replaced with a bond to a carbon atom. Therefore, substituted cycloalkyl, aryl, heterocycle and heteroaryl groups may also be substituted with substituted or unsubstituted alkyl, alkenyl, and alkynyl groups as defined below.

Alkyl groups include straight chain and branched alkyl groups having from 1 to about 20 carbon atoms, and typically from 1 to 12 carbons or, in some embodiments, from 1 to 8, 1 to 6, or 1 to 4 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. As stated above, the present disclosure is understood to include embodiments where, for instance a “substituted alkyl” optionally contains one or more alkene and/or alkyne.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-di-substituted cyclohexyl groups, which may be substituted with substituents such as those listed above. In some embodiments, a cycloalkyl group has one or more alkene bonds, but is not aromatic.

Heterocycle groups include aromatic (also referred to as heteroaryl) and non-aromatic ring compounds containing 3 or more ring members, of which one or more is a heteroatom such as, but not limited to, N, O, S or B. In some embodiments, heterocycle groups include 3 to 20 ring members, whereas other such groups have 3 to 6, 3 to 10, 3 to 12, or 3 to 15 ring members. Heterocycle groups encompass unsaturated, partially saturated and saturated ring systems, such as, for example, imidazolyl, imidazolinyl and imidazolidinyl groups. The phrase “heterocycle group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocycle groups that have other groups, such as alkyl, oxo or halo groups, bonded to one of the ring members. Rather, these are referred to as “substituted heterocycle groups”. Heterocycle groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocycle groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, S or B. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”) as used herein refers to —NHR* and —NR*R* groups, wherein R* are independently hydrogen, or a substituted or unsubstituted alkyl, alkenyl, alkynyl, cycloalkyl, aryl, aralkyl, heterocyclylalkyl or heterocycle group as defined herein. In some embodiments, the amine is NH₂, methylamino, dimethylamino, ethylamino, diethylamino, propylamino, isopropylamino, phenylamino, or benzylamino. In some embodiments, the two R* groups together with the nitrogen to which they are attached form an optionally substituted heterocyclic ring. In further embodiments, the optionally substituted ring is an optionally substituted piperazine, optionally substituted piperidine, or optionally substituted pyrrolidine.

The term “amide” refers to a —NR*R*C(O)— group wherein R* each independently refer to a hydrogen, (C₁-C₈)alkyl, or (C₃-C₆)aryl or the two R* together with the nitrogen to which they are attached form an optionally substituted heterocyclic ring.

The present technology, thus generally described, will be understood more readily by reference to the following examples, which are provided by way of illustration and are not intended to be limiting of the present technology. In some embodiments, the synthetic protocols and strategies presented herein provide deuterated compounds. In some embodiments, the synthetic protocols and strategies presented herein provide undeuterated compounds. In some embodiments, the synthetic protocols and strategies presented herein are adapted to provide deuterated or undeuterated compounds. In some embodiments, the synthetic protocols and strategies presented herein provide or are adapted to provide deuterated or undeuterated squalamine, or deuterated or undeuterated squalamine derivatives (such as but not limited to aminosterol 1436).

### EXAMPLES

### Example 1. Compound 34 from BDG-4

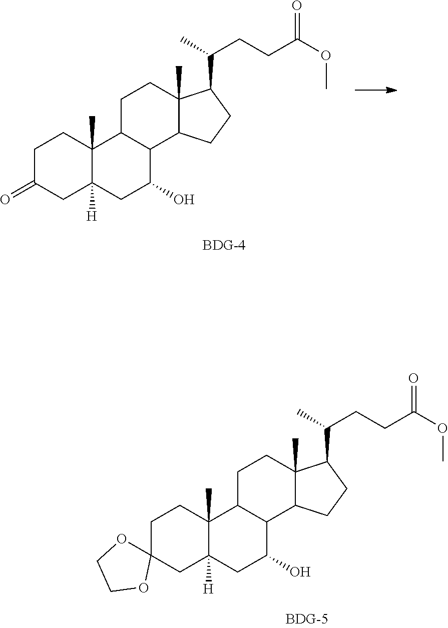 [SMILES: COC(=O)CC[C@@H](C)[C@H]1CCC2C3C(CC[C@@]21C)[C@@]1(C)CCC(=O)C[C@@H]1C[C@H]3O ; COC(=O)CC[C@@H](C)[C@H]1CCC2C3C(CC[C@@]21C)[C@@]1(C)CCC2(C[C@@H]1C[C@H]3O)OCCO2]

Preparation of BDG-5.

BDG-4 (370.54 g, 916.3 mmol) was dissolved in dichloromethane (DCM, 3,600 mL) and treated with ethylene glycol (210 mL, 3.77 mol, 4.11 eq.) along with p-toluenesulfonic acid-hydrate (36.27 mmol, 0.04 eq.). The mixture was refluxed until the reaction was complete (TLC in 70:30 hexane:acetone showed ca 3-5% residual starting material that did not change). After about 4 h reflux, the mixture was cooled, treated with of 10% potassium carbonate (150 mL), and the layers separated. The DCM was re-extracted with 800 mL of 5% sodium chloride solution and 10 mL of 10% potassium bicarbonate solution. The two aqueous layers were back-extracted with 500 mL of DCM. The combined DCM extracts were dried over magnesium sulfate, filtered, washed with DCM (2×100 mL), and concentrated under vacuum to afford a solid, which was slurried in acetonitrile (4.8 L) containing triethylamine (2 mL) at 55° C. for 1 h, then 35° C. for 1 hour, and finally at 12° C. for 2 h. The product was filtered and washed with cold acetonitrile (2×100 mL) containing triethylamine (0.1 mL). The crystals were dried at 50° C. in the vacuum oven (0.2 mm) overnight to afford BDG-5 (351.0 g, 85%); m.p. was 175.6-177.5° (at 20/minute from ca 1400); ¹H NMR (500 MHz, CDCl₃) δ 3.93 (m, 4H), 3.82 (br s, 1H), 3.66 (s, 3H), 2.4-2.2 (m, 2H), 1.9-1.2 (m, 24H), 0.92 (d, 3H, J=7 Hz), 0.81 (s, 3H), 0.66 (s, 3H); ¹³C NMR (CDCl₃) δ 177,109, 67.9, 64.2, 64.1, 55.8, 51.4, 50.5, 45.6, 42.6, 39.5, 39.4, 37.5, 36.3, 36.1, 35.7, 35.5, 35.4, 31.2, 31.0, 30.9, 28.0, 23.6, 20.9, 18.3, 11.8, 10.4.

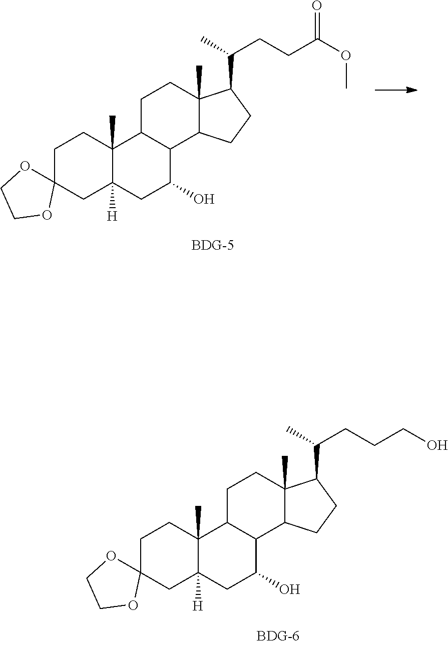 [SMILES: COC(=O)CC[C@@H](C)[C@H]1CCC2C3C(CC[C@@]21C)[C@@]1(C)CCC2(C[C@@H]1C[C@H]3O)OCCO2 ; C[C@H](CCCO)[C@H]1CCC2C3C(CC[C@@]21C)[C@@]1(C)CCC2(C[C@@H]1C[C@H]3O)OCCO2]

Preparation of BDG-6.

Lithium aluminum hydride (7.44 g, 196.05 mmol, pellets) was added to anhydrous THF (435 mL) under nitrogen and stirred overnight at 20° C. to break up the pellets. The suspension was cooled to 10° C. and a solution of BDG-5 (77.57 g, 172.91 mmol) was added dropwise over 160 min. The mixture was stirred 1 h further, and was quenched by adding 2 mL of ethyl acetate, followed by dropwise addition of 20% aqueous potassium hydroxide solution (7.44 mL) over 10 min. The mixture became very thick, then thinned out and became more granular with additional stirring for 15 h at 20° C. The mixture was filtered through CELITE® 545 to remove the aluminum salts. The filter cake was washed with of THF (5×90 mL) to ensure that all the product was obtained in the filtrate, which was concentrated to afford crude BDG-6 (82.01 g, theory=72.7 g). In practice in larger runs, the THF solution was concentrated to dryness, and re-concentrated with DCM to remove traces of water prior to next step; ¹H NMR (500 MHz, CDCl₃) δ 3.93 (m, 4H), 3.82 (br s, 1H), 3.61 (m, 2H), 1.97-1.84 (m, 4H), 1.7-1.0 (m, 22H), 0.93 (d, 3H, J=7 Hz), 0.81 (s, 3H), 0.66 (s, 3H); ¹³C NMR (CDCl₃) δ 109.3, 67.9, 64.15, 64.13, 63.5, 56.0, 50.6, 45.6, 42.6, 39.51, 39.48, 37.5, 36.3, 36.1, 35.7, 35.6, 35.5, 31.8, 31.2, 29.4, 28.2, 23.6, 20.9, 18.6, 11.8, 10.4.

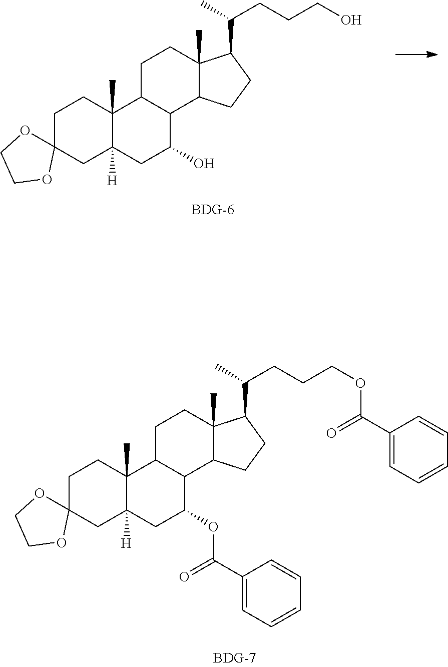 [SMILES: C[C@H](CCCO)[C@H]1CCC2C3C(CC[C@@]21C)[C@@]1(C)CCC2(C[C@@H]1C[C@H]3O)OCCO2 ; C[C@H](CCCOC(=O)c1ccccc1)[C@H]1CCC2C3C(CC[C@@]21C)[C@@]1(C)CCC2(C[C@@H]1C[C@H]3OC(=O)c1ccccc1)OCCO2]

Preparation of BDG-7.

BDG-6 (214.55 g, 510.1 mmol) was dissolved in DCM (3.0 L) and treated with 4-dimethylaminopyridine (250.3 g, 2.048 mol) and then benzoyl chloride (180 mL, 217.98 g, 1.551 mol) dropwise. An exotherm to 31° C. was noted after about 40 mL of benzoyl chloride was added. The temperature was held to 25° C. for the remainder of the addition which took a total of 35 min. The solution was stirred overnight at 25° C., diluted with DCM (200 mL), and treated with 10% aqueous potassium bicarbonate (2 L) generating a small amount of carbon dioxide. The layers were separated and the aqueous layer was re-extracted with DCM (500 mL). The total organic layers were dried over magnesium sulfate and concentrated to afford BDG-7 as a solid (163.6 g, 100%). In practice in larger runs, the dibenzoate solution was concentrated to a suitable volume for the next step. ¹H NMR (500 MHz, CDCl₃) δ 8.07 (d, 2H, J=8 Hz), 8.00 (d, 2H, J=8 Hz), 7.57 (t, 1H, J=8 Hz), 7.53 (t, 1H, J=8 Hz), 7.47 (t, 2H, J=8 Hz), 7.41 (t, 2H, J=8 Hz), 5.16 (br s, 1H), 4.25 (m, 2H), 3.87 (m, 4H), 2.02-1.85 (m, 2H), 1.8-1.1 (m, 22H), 0.95 (d, 3H, J=7 Hz), 0.88 (s, 3H), 0.68 (s, 3H); ¹³C NMR (CDCl₃) δ 166.7, 166.0, 132.8, 131.1, 130.5, 129.7, 129.5, 128.4, 128.3, 109.0, 71.9, 65.5, 64.2, 64.1, 55.8, 50.7, 47.2, 42.8, 39.5, 38.6, 37.4, 37.2, 35.7, 35.5, 35.3, 33.3, 32.0, 31.2, 28.0, 25.2, 23.6, 21.1, 18.6, 11.8, 10.5.

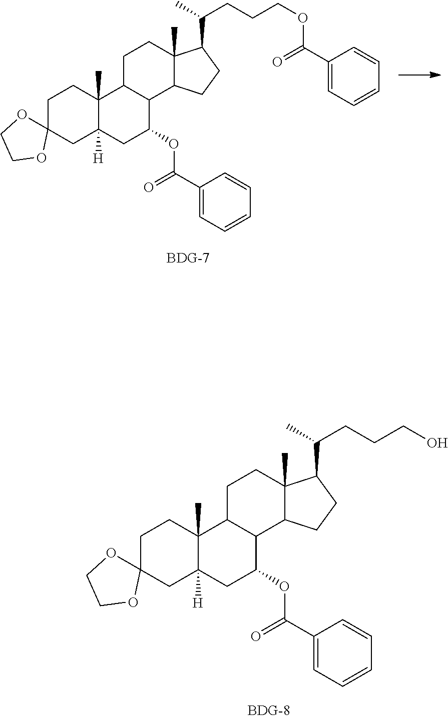 [SMILES: C[C@H](CCCOC(=O)c1ccccc1)[C@H]1CCC2C3C(CC[C@@]21C)[C@@]1(C)CCC2(C[C@@H]1C[C@H]3OC(=O)c1ccccc1)OCCO2 ; C[C@H](CCCO)[C@H]1CCC2C3C(CC[C@@]21C)[C@@]1(C)CCC2(C[C@@H]1C[C@H]3OC(=O)c1ccccc1)OCCO2]

Preparation of BDG-8.

BDG-7 (22.13 kg, 35.2 mol) was dissolved in 1:1 tetrahydrofuran:methanol solution (220 L) at 25° C. and treated with a solution of 50% sodium hydroxide (3.7 L, 70.4 mol, 2.0 eq.) in deionized water (11 L) and stirred for 4 h. An aliquot (0.1 mL) of the reaction mixture was partitioned between ethyl acetate (0.5 mL each) and 1 M potassium bicarbonate solution. The organic layer was analyzed by TLC (70:30 hexane:acetone) and the reaction was judged complete. An aqueous solution of potassium bisulfate was prepared by mixing deionized water (23 L), 96% sulfuric acid (3.7 L, 66.6 moles), and 45% potassium hydroxide (5.7 L, 66.7 moles). The resulting solution of potassium bisulfate was added via sprayball to the reaction mixture to a final pH of 8.39 (pH meter). The mixture was vacuum-distilled to about 60 L volume; and treated with of ethyl acetate (100 L) and water (100 L). The 2-phase mixture was agitated for 10 min, and the layers separated over about 20 min. The aqueous phase was re-extracted with 37 L of ethyl acetate. The total ethyl acetate layer (about 160 L) was vacuum concentrated to a volume of 80 L at 50° C. Another 50 L of ethyl acetate was added, and the solution dried over anhydrous sodium sulfate (15 kg) by stirring overnight. The solution was filtered over CELITE® 545 to remove the sodium sulfate. The filter cake and the reactor were rinsed with 2×37 L of ethyl acetate. The dried ethyl acetate solution was vacuum concentrated from about 225 L volume to 40 L. Acetonitrile (50 L) was added and the solution was re-distilled to remove ethyl acetate. A second and third batch of acetonitrile (50 and 100 L) were added and evaporated. Finally, 100 L of acetonitrile was added and this solution was counter-currently extracted with 3×104 L of hexane to remove most of the methyl benzoate. The 3 hexane extracts were re-extracted with a second 104 L of acetonitrile. The hexane layers were checked by TLC for BDG-8 intermediate and none was present. The acetonitrile (ca. 300 L) was vacuum concentrated to ca. 50 L volume. Isopropanol (57 L) was added and concentrated to ca. 50.L; this operation was repeated 2 more times to remove all the acetonitrile. The volume was adjusted to 100 L with isopropanol and the solution slowly cooled to 0° C. and was seeded with crystalline BDG-8 to initiate crystallization. The mixture was slowly cooled to −20° C. overnight. The crystal slurry was filtered through a jacketed filter at −20° C. and rinsed with 10 L of −20° C. isopropanol. The filter cake was dried by vacuum for several h at −20° C., followed by letting the filter warm slowly to room temperature (about 18° C.) and then drying with warm nitrogen yielding 11.92 kg of BDG-8. The mother liquors were chromatographed to isolate additional product. A column of silica gel (19 kg) was packed in methylene chloride. The filtrate of the product was vacuum concentrated to dryness and ½ was added to the column in methylene chloride. Elution with 100 L of methylene chloride (DCM) gave the product. The column was flushed with 20 L of 85:15 methylene chloride:ethyl acetate, then 20 L of 60:40 methylene chloride:ethyl acetate, and finally back to methylene chloride (40 L). The second 2 of the mother liquors was similarly chromatographed to give a total of 5.32 kg of pure BDG-8 (Total yield of BDG-8: 17.24 kg, 93.3%). ¹H NMR (500 MHz, CDCl₃) δ 8.07 (d, 2H, J=8 Hz), 7.57 (t, 1H, J=7 Hz), 7.48 (t, 2H, J=7 Hz), 5.15 (br s, 1H), 3.88 (m, 4H), 3.56 (br s, 2H), 2.02-1.84 (m, 2H), 1.75-0.98 (m, 24H), 0.92 (d, 3H, J=6.5 Hz), 0.88 (s, 3H), 0.67 (s, 3H). ¹³C NMR (CDCl₃) δ 166.0, 132.7, 131.0, 129.7, 128.4, 109.0, 71.9, 64.2, 64.1, 63.5, 55.8, 50.7, 47.2, 42.7, 39.5, 38.6, 37.3, 37.2 35.7, 35.5, 33.3, 31.7, 31.3, 29.3, 28.0, 23.6, 21.1, 18.6, 11.8, 10.5.

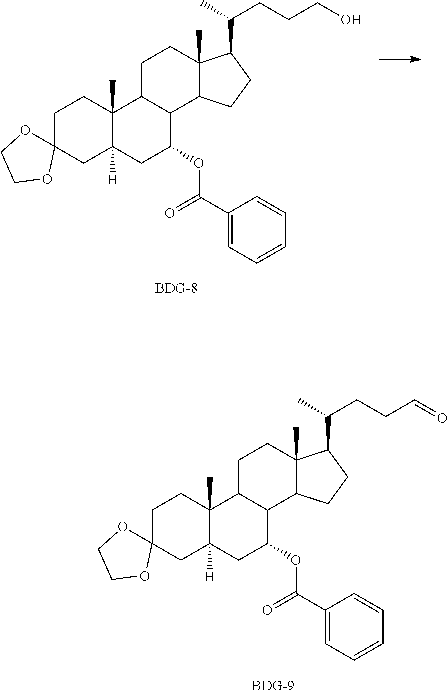 [SMILES: C[C@H](CCCO)[C@H]1CCC2C3C(CC[C@@]21C)[C@@]1(C)CCC2(C[C@@H]1C[C@H]3OC(=O)c1ccccc1)OCCO2 ; C[C@H](CCC=O)[C@H]1CCC2C3C(CC[C@@]21C)[C@@]1(C)CCC2(C[C@@H]1C[C@H]3OC(=O)c1ccccc1)OCCO2]

Preparation of BDG-9.

A 50 L jacketed reactor under nitrogen was charged with potassium bromide (250 g, 2.23 mol), sodium bicarbonate (265 g, 3.15 mol), and water (5 L); agitated and cooled with a −5° C. jacket. To the reactor was added BDG-8 (5.32 kg, 10.14 mol) dissolved in ˜13 L of dichloromethane, followed by rinse from 8 L of dichloromethane. When the reaction mixture attained a temperature <5° C., TEMPO (50 g, 0.286 mole) was added through the manhole. The reactor was then charged via pump with 12.5% bleach (5.5 kg), keeping the mixture <5° C. The reaction was sampled to estimate additional amount of bleach needed. Bleach continued to be added in small portions while sampling periodically until reaction was judged to be complete (<2% BDG-8 remaining; a total of 6.2 kg of bleach was added). A solution of sodium thiosulfate (200 g) in water (1 L) was added with vigorous stirring until a negative starch/iodide test was obtained. Hexane (20 L) was added to the reactor with stirring and the layers settled. The lower aqueous phase was drained, and the organic layer was washed with saturated aqueous potassium bicarbonate (5 L). The organic phase was vacuum filtered through a pad of sodium sulfate/silica gel (1 kg of each) in a sintered glass filter funnel and rinsed with hexane/dichloromethane (1/1), dichloromethane, and finally 5% methyl-t-butyl ether (MTBE) in dichloromethane. The combined filtrates were evaporated in two batches in a Buchi apparatus at 40° C. Hexane was added to each batch and evaporated again until a thick slurry formed. The solids were filtered, washed with hexane, and dried in a vacuum oven to afford BDG-9 (4.58 kg). The filtrates and washings were combined and concentrated to get a second crop, which was filtered and washed with 5% MTBE/hexane, then hexane, and dry to get another 565 g of product for a total yield of 5.145 kg (97% yield) of BDG-9. The material contains about 1.5% of residual starting material, but no detectable (NMR) carboxylic acid. ¹H NMR (500 MHz, CDCl₃) δ 9.72 (s, 1H), 8.07 (d, 2H, J=7 Hz), 7.59 (t, 1H, J=7 Hz), 7.49 (t, 2H, J=7 Hz), 5.16 (br s, 1H), 3.88 (m, 4H), 2.45-2.27 (m, 2H), 2.00-1.85 (m, 2H), 1.78-1.17 (m, 22H), 0.913 (d, 3H, J=Hz), 0.88 (s, 3H), 0.68 (s, 3H). ¹³C NMR (CDCl₃) δ 203, 166, 132.8, 131.0, 129.8, 128.4, 109, 72.0, 64.17, 64.11, 55.7, 50.7, 47.2, 42.8, 40.8, 39.5, 38.6, 37.3, 37.2, 35.7, 35.5, 35.4, 33.3, 31.2, 27.9, 27.8, 23.7, 21.1, 18.3, 11.7, 10.5.

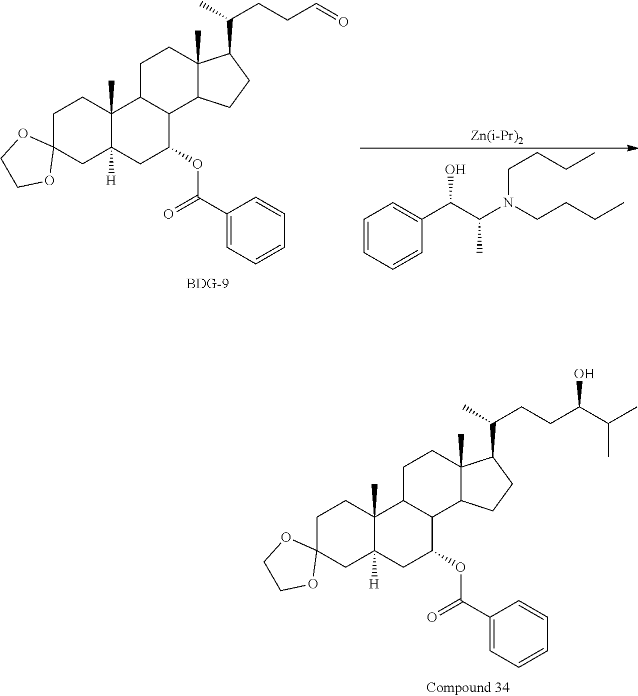 [SMILES: C[C@H](CCC=O)[C@H]1CCC2C3C(CC[C@@]21C)[C@@]1(C)CCC2(C[C@@H]1C[C@H]3OC(=O)c1ccccc1)OCCO2 ; CCCCN(CCCC)[C@H](C)[C@@H](O)c1ccccc1 ; CC(C)[C@H](O)CC[C@@H](C)[C@H]1CCC2C3C(CC[C@@]21C)[C@@]1(C)CCC2(C[C@@H]1C[C@H]3OC(=O)c1ccccc1)OCCO2 ; "Zn(i-Pr)2"]

Preparation of Compound 34.

A 1.54 M solution of diisopropyl zinc in o-xylene (˜12.5 L, ˜19.2 mol) was charged into a 50 L jacketed reactor under nitrogen and the chiller was set to −10° C. (maintain N₂ purge). After starting agitation, a small sample was removed from the reactor to assess the diisopropyl zinc titer and adjust charges if necessary. When the mixture reached −5° C., the chiral ligand ((1S,2R)-2-(dibutylamino)-1-phenylpropan-1-ol) (285 g, 1.08 mol) was added at a rate to maintain internal temperature <5° C., followed by a rinse with toluene. After cooling the mixture back down to −5° C., BDG-9 (4.6 kg, 8.80 mol) in toluene (8 L) was charged via pump at rate to keep mixture <0° C., followed by a toluene rinse (0.5 L). The reactor was stirred at −5° C. for at least 16 h and a sample was taken for analysis by TLC to determine completion. If reaction was not complete, stirring and/or temperature was increased. When reaction was deemed complete, the reactor was charged with acetic acid (1 L) dissolved in MTBE (1 L) through an addition funnel at rate to keep mixture <10° C. and control off-gassing. MTBE (18 L) was charged to the reactor through the manhole, followed by saturated aqueous ammonium chloride (NH₄Cl, 18 L) at a rate to keep mixture <10° C. and control off-gassing. The reaction mixture was settled and if aqueous layer is below pH 7, add aqueous potassium bicarbonate (KHCO₃). The layers were separated and the organic layer was vacuum filtered through a pad of sodium sulfate (2 kg) on top of silica gel (4 kg) in a sintered glass filter funnel, rinsing through with MTBE. The combined filtrates were divided into two portions, evaporated in a Buchi flask at 40-60° C. (bath temp.) to remove almost all of the MTBE and toluene, and some of the xylene. Upon cooling, the contents of each flask solidified and were treated with acetonitrile (5 L), heated to 55° C. for 1 h while spinning, cooled back to room temperature, collected by vacuum filtration, and washed with 1.5 L of acetonitrile. The solid was dried on filters, air dried in trays, and finally placed in vacuum oven at 40-60° C. to give Compound 34 (2.408 kg) pure enough to submit to the crystallization procedure. The mother liquors were combined and evaporated to afford a waxy solid. The solid was dissolved in a minimum volume of DCM and transferred to a 20 L reactor, rinsed with MTBE, diluted with hexane (5 L), extracted with 200 g of citric acid dissolved water (4 L) and again with water (2 L). The combined aqueous extract was washed with MTBE, which was combined with the organic phase with that from above. The ligand could be recovered from the aqueous layer by adding 10% sodium hydroxide solution to pH 12, washing aqueous layer with MTBE (3 L), filtering through sodium sulfate, and evaporating to afford recovered chiral ligand (203 g, 71% recovery). The combined organic layers were filtered through a bed of sodium sulfate, evaporated, dissolved in DCM, added to a column of silica gel (20 kg), and eluted with DCM and then with 3-5% MTBE in DCM. Fractions containing pure Compound 34 were pooled and evaporated. Although batch integrity was lost due to mixing of some streams from different lots, a total of 6.09 kg (10.7 mol) of Compound 34 has been isolated from 8.6 kg (16.4 mol) of BDG-9 input, for a yield of 65%.

Recrystallization of Compound 34 (4,146 g) was achieved by stirring in a 25 L reactor with 18.2 L of ethyl acetate, warmed to 65-69° C. to achieve a solution, cooled in 30 min to 40° C., seeded with pure material, cooled at a rate of 10° C./45 min to −20° C., and maintained for a minimum of 5 h. A suitable-sized jacketed filter was cooled to −20° C. and the product slurry was vacuum-filtered at −20° C., and subsequently washed with of ethyl acetate (2×800 mL precooled to −20° C.), dried on filter, then allowed to warm to rt under vacuum. The product was transferred to glass trays and dried in a vacuum oven at 45° C. for a few hours, then at 60° C. overnight (vacuum was about 0.2-0.5 mm) to afford pure Compound 34 (2,400 g). The mother liquors were concentrated to dryness to give 1.72 kg. This was chromatographed on 20 kg of silica gel with 97:3 methylene chloride:MTBE to give 1.1 kg of purified material. This was recrystallized from ethyl acetate following the above procedure to give another 849 grams of final product Compound 34 (Total: 3,249 g, 78%); HPLC de=97.3%, detection=232 nm, column Siliachrom dt C18, 5 micron particle size, 250×4.6 mm, elution 80% acetonitrile: water to 95% acetonitrile at 0.9 mL/min over 40 minutes, 24-S-isomer (30.7 min), desired 24-R-isomer (31.5 min); mp (DSC) 218.4-219.6° C.; ¹H NMR (500 MHz, CDCl₃) δ 8.07 (d, 2H, J=7 Hz), 7.58 (t, 1H, J=7 Hz), 7.48 (t, 2H, J=7 Hz), 5.16 (br s, 1H), 3.87 (m, 4H), 3.27 (br s, 1H), 2.07-1.84 (m, 2H), 1.8-1.1 (m, 25H), 0.93-0.87 (m, 12H), 0.78 (s, 3H); ¹³C NMR (CDCl₃) δ 168, 133, 131, 129.7, 128.4, 109, 77.0, 71.9, 64.2, 64.1, 55.9, 50.7, 47.3, 42.7, 39.5, 38.6, 37.3, 37.2, 35.7, 35.6, 35.4, 33.5, 33.2, 32.0, 31.3, 30.6, 28.1, 23.6, 21.1, 18.9, 18.6, 17.2, 11.8, 10.5.

### Example 2: Compound 34 from Unprotected Steroid

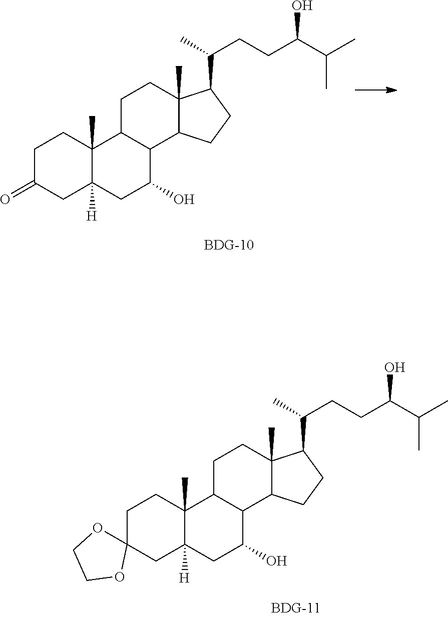 [SMILES: CC(C)[C@H](O)CC[C@@H](C)[C@H]1CCC2C3C(CC[C@@]21C)[C@@]1(C)CCC(=O)C[C@@H]1C[C@H]3O ; CC(C)[C@H](O)CC[C@@H](C)[C@H]1CCC2C3C(CC[C@@]21C)[C@@]1(C)CCC2(C[C@@H]1C[C@H]3O)OCCO2]

Preparation of Compound BDG-11.

BDG-10 (Commercially available from Bridge Organics) containing 0.5 eq of water (1.714 g, 4.007 mmol) was dissolved in anhydrous acetonitrile (32 mL) and treated with anhydrous oxalic acid (101.8 mg, 1.13 mmol, 0.28 eq). Anhydrous ethylene glycol (0.30 mL, 5.38 mmol, 1.34 eq) was added, and the solution was stirred at 54° C. for 1.5 h. Because the reaction was not complete by NMR, more oxalic acid (46 mg, 0.51 mmol, 0.128 eq) and ethylene glycol (0.10 ml, 1.82 mmol, 0.295 eq) was added. After stirring the solution over the weekend, it was concentrated to about half the volume, and diluted with ethyl acetate (50 mL). The organic layer was extracted with 10% aqueous potassium bicarbonate solution (10 mL), and then with aqueous 25% sodium chloride solution (10 mL). The aqueous layers were back-extracted with ethyl acetate (25 mL). The combined organic layers were dried over magnesium sulfate, filtered, washed with ethyl acetate (2×25 mL), and concentrated to dryness under vacuum to yield BDG-11 (1.885 g) containing 7% residual starting material by NMR. ¹H NMR (500 MHz, CDCl₃) δ 3.93 (s, 4H), 3.82 (br s, 1H), 3.32 (m, 1H), 1.97-1.87 (m, 3H), 1.72-1.08 (m, 24H), 0.93 (m, 9H), 0.81 (s, 3H), 0.66 (s, 3H).

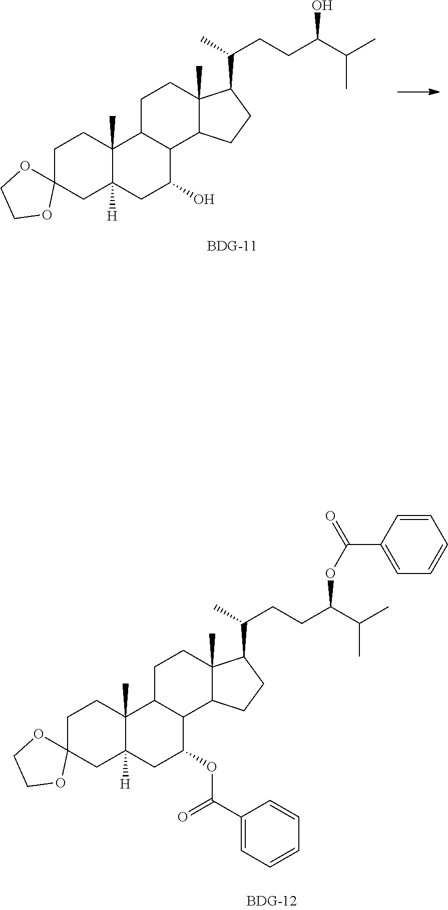 [SMILES: CC(C)[C@H](O)CC[C@@H](C)[C@H]1CCC2C3C(CC[C@@]21C)[C@@]1(C)CCC2(C[C@@H]1C[C@H]3O)OCCO2 ; CC(C)[C@@H](CC[C@@H](C)[C@H]1CCC2C3C(CC[C@@]21C)[C@@]1(C)CCC2(C[C@@H]1C[C@H]3OC(=O)c1ccccc1)OCCO2)OC(=O)c1ccccc1]

Preparation of BDG-12.

BDG-11 (1.875 g, 4.05 mmol) was dissolved in anhydrous dichloromethane (DCM, 50 mL) and 4-dimethylaminopyridine (3.00 g, 24.55 mmol, 6.06 eq) was added. Benzoyl chloride (2.44 mL, 2.955 g, 21.02 mmol, 5.19 eq) was added and the solution was stirred for 4 h. TLC of an aliquot indicated completion after 3 hours. DCM (50 mL) was added, and the solution was extracted with of 10% aqueous potassium bicarbonate (2×50 mL). The aqueous layers were back-extracted with DCM (50 mL) and the combined DCM layers were dried over magnesium sulfate, filtered, washed with DCM (2×25 mL), and concentrated under vacuum to afford a solid containing 4-dimethylaminopyridine (4.25 g). The product was purified by dissolving in DCM and adding to a silica gel column (62 g of silica gel, 2.5×23 cm) and eluted, collecting 200 mL fractions. Fractions 2-4 were combined and concentrated to dryness under vacuum to afford BDG-12 (2.685 g, 100%). ¹H NMR (500 MHz, CDCl₃) δ 8.04 (d, 2H, J=6 Hz), 8.0 (d, 2H, J=6 Hz), 7.59-7.51 (m, 2H), 7.49-7.38 (m, 4H), 5.13 (br s, 1H), 4.92 (m, 1H), 3.91-3.84 (m, 4H), 1.99-1.84 (m, 3H), 1.74-1.01 (m, 24H), 0.96-0.90 (m, 9H), 0.87 (s, 3H), 0.65 (s, 3H).

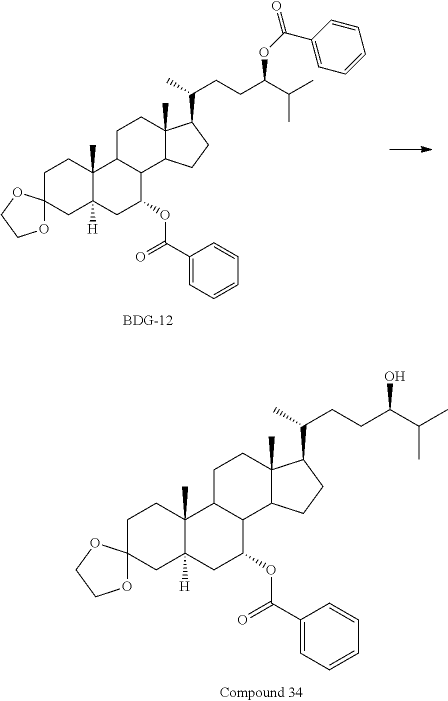 [SMILES: CC(C)[C@@H](CC[C@@H](C)[C@H]1CCC2C3C(CC[C@@]21C)[C@@]1(C)CCC2(C[C@@H]1C[C@H]3OC(=O)c1ccccc1)OCCO2)OC(=O)c1ccccc1 ; CC(C)[C@H](O)CC[C@@H](C)[C@H]1CCC2C3C(CC[C@@]21C)[C@@]1(C)CCC2(C[C@@H]1C[C@H]3OC(=O)c1ccccc1)OCCO2]

Preparation of Compound 34.

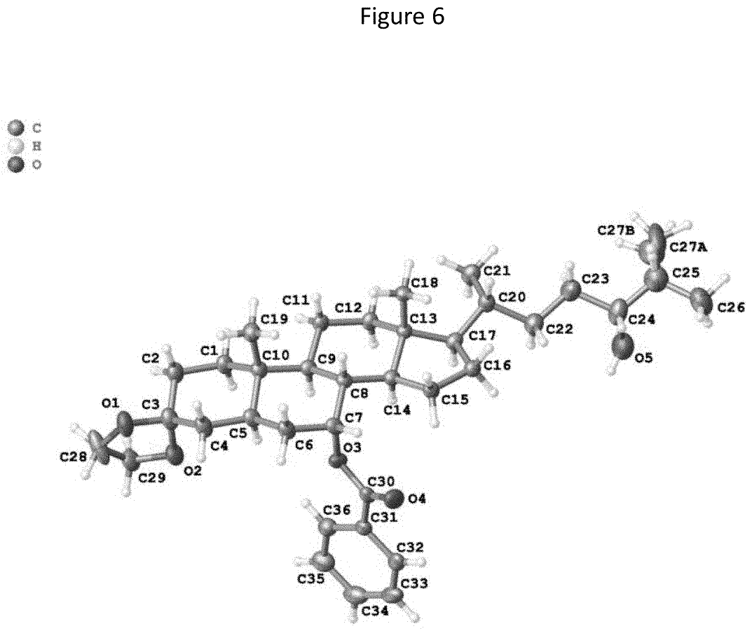

The BDG-12 (2.600 g, 3.875 mmol) was dissolved in methanol (100 mL), THF (35 mL), and 50% aqueous sodium hydroxide solution (3.00 mL, 56.79 mmol, 14.66 eq), and was stirred at 25° C. TLC estimations at 6 h reaction was 15% conversion; 23 h, 39% conversion; 53 h, 72% conversion and 28% starting material (confirmed by NMR)—no trace of C7 ester hydrolysis was observed. The mixture was added to 5% potassium bicarbonate solution (250 mL) and was extracted with ethyl acetate (2×125 mL). The ethyl acetate layers were re-extracted with 5% aq. potassium bicarbonate solution (250 mL), then with 25% sodium chloride solution (125 mL). The organics layer was dried over magnesium sulfate, filtered, washed with ethyl acetate (2×50 mL), and concentrated to dryness under vacuum to afford solid (2.18 g), which was chromatographed on 150 g of silica gel and eluted with 95/5/0.5 DCM/ethyl acetate/concentrated ammonium hydroxide, collecting with fifty mL fractions. The starting material was eluted first in fractions 7-13 (545 mg, 20%) and the Compound 34 eluted in fractions 15-41 (1.303, 72%). Crystallization: Compound 34 (1.235 g) was dissolved in (100/0.2 methanol/triethylamine) and heated to 60° C. to achieve solution. After slow cooling to 0° C. and stirring overnight at 00 Compound 34 (862 mg) was obtained. Recrystallization from spectroscopy grade methyl t-butyl ether gave crystals (664 mg) that were quite pure. NMR and HPLC data were taken and were consistent with the Compound 34 prepared in Example 1. Slow recrystallization of 30 mg from spectroscopy grade hexane (0.25 mL) gave excellent needle crystals suitable for X-ray crystallography (FIG. 6), confirming the (R)-stereochemistry at C24. Compound 34 is useful in the preparation of squalamine and 1436.

### Example 3: Preparation of D₇-1436 and Squalamine-D₇

### Example 3a: Preparation of Compound D₇-1436

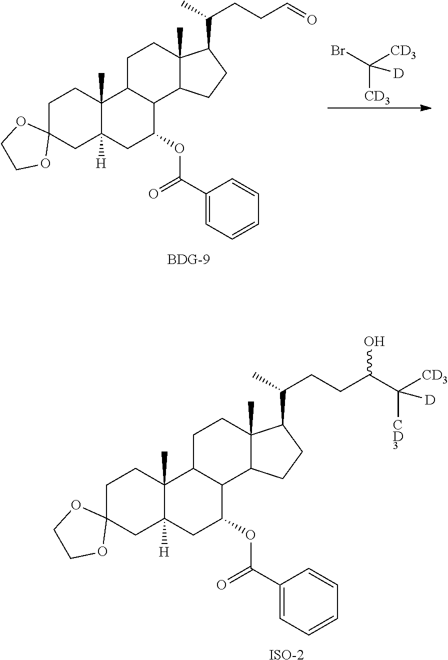

Preparation of ISO-2.

To refluxing THF (30 mL) in a round bottom flask fitted with a condenser was added magnesium turnings (0.312 g, 13 mmol), along with 2 small iodine pellets. After 15 min of stirring, 2-bromopropane-D₇ (1.2 mL, 12 mmol) was slowly added. The reaction was refluxed under nitrogen for 50 min, until only excess magnesium remained in the reaction vessel. In a separate flask, BDG-9 (2.0 g, 3.8 mmol) was dissolved in THF (35 mL) at 0° C. and treated with the D₇-isopropyl Grignard solution. After 30 min of stirring, TLC showed no starting material remained and the reaction mixture was brought to room temperature and quenched with methanol (2 mL). A portion of the THF was evaporated off, and the remaining solution was poured into water, extracted with diethyl ether, washed with NaHSO₄ and brine, dried over Na₂SO₄, and evaporated to give ISO-2 (2.12 g, 3.6 mmol, 95%); ¹H NMR (CDCl₃, 300 MHz) δ 8.02-7.98 (m, 2H), 7.52-7.49 (m, 1H), 7.44-7.38 (m, 2H), 5.09 (br s, 1H), 3.80 (m, 4H), 3.21 (m, 1H), 1.9-1.0 (m, 26H), 0.85 (d, 3H, J=6 Hz), 0.81 (s, 3H), 0.61 (s, 3H).

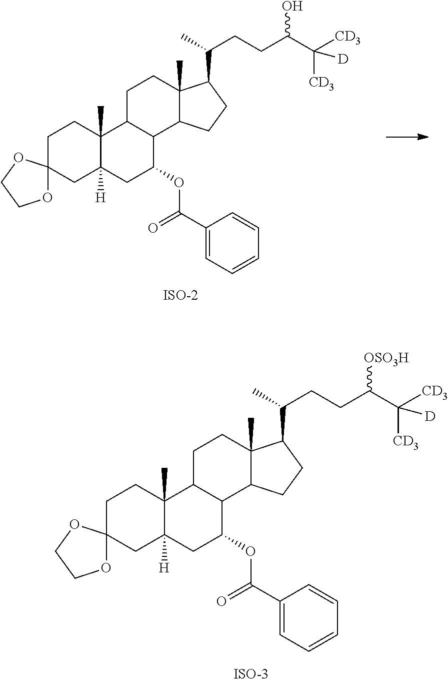

Preparation of ISO-3.

ISO-2 (2.12 g, 3.6 mmol) was dissolved in anhydrous pyridine (30 mL), and sulfur trioxide—pyridine complex (1.14 g, 7.2 mmol) was added while stirring under nitrogen. The solution was stirred overnight, and TLC indicated that no starting material remained, with the product sitting at baseline in 35% ethyl acetate/hexanes. The reaction was extracted in chloroform, washed with dilute NaHSO₄ solution and brine, dried over Na₂SO₄, filtered and evaporated to give ISO-3 (2.23 g, 3.4 mmol, 94%); ¹H NMR (DMSO-D₆, 300 MHz) δ 8.00-7.97 (m, 2H), 7.67-7.64 (m, 1H), 7.58-7.53 (m, 2H), 5.08 (br s, 1H), 3.77 (m, 1H), 3.5 (m, 4H), 2.4-1.0 (m, 26H), 0.88 (d, 3H, J=6 Hz), 0.85 (s, 3H), 0.68 (s, 3H).

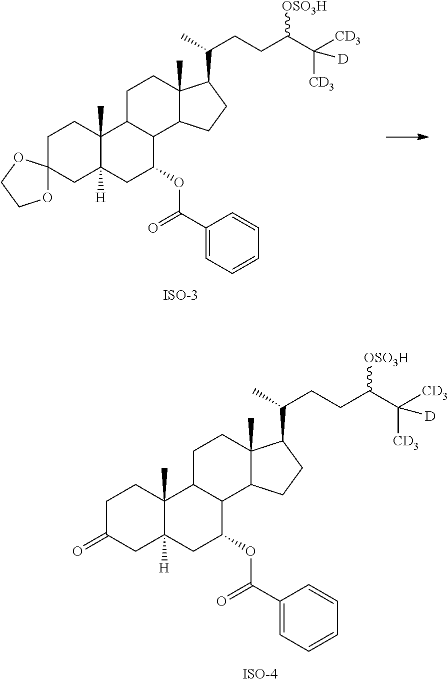

Preparation of ISO-4.

ISO-3 (2.23 g, 3.4 mmol) was dissolved in acetone and acidic AMBERLYST® Ion-Exchange Resin (1.5 g) of was added. After stirring the solution overnight under nitrogen, TLC in 10% acetone/DCM showed the reaction was complete. The solution was filtered through CELITE® and evaporated to give ISO-4 (2.05 g, 3.4 mmol, 100%).

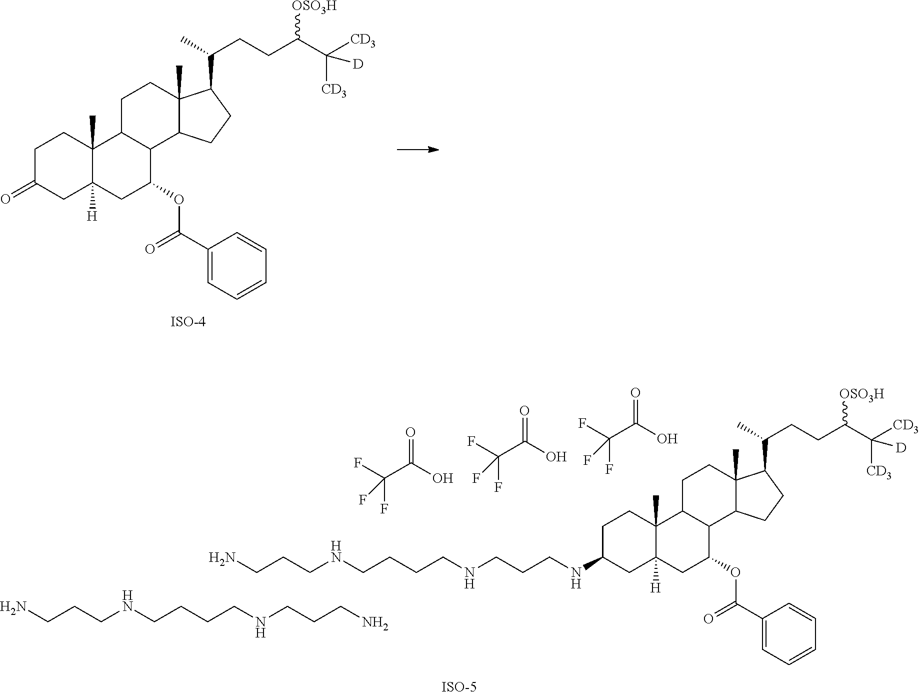

Preparation of ISO-5.

ISO-4 (0.200 g, 0.33 mmol) was dissolved in anhydrous methanol (25 mL) and charged with 3 equivalents of spermine free base (193 mg, 0.95 mmol). This was stirred at reflux overnight, with an addition funnel filled with molecular sieves attached directly to the reaction flask, so as to capture water from the refluxing reaction and encourage formation of the imine intermediate. After overnight stirring, the reaction was cooled to −78° C., and charged with 3 equivalents of sodium borohydride (0.035 g) in small portions. TLC in 6:3:1 CHCl₃/MeOH/NH₄OH showed that the reaction was complete. Water (10 mL) was added, and most of the methanol was evaporated off. The solution was acidified with trifluoroacetic acid and poured onto a column of washed AMBERCHROME®. The column was flushed with 0.1% TFA in H₂O to remove leftover salts from the reductive amination, and then a stepwise gradient of H₂O/acetonitrile (both 0.1% TFA) was used to flush the product from the column. The fractions were checked by TLC with ninhydrin stain, and the appropriate fractions were combined and lyophilized overnight. The resulting powdery crystalline material was dissolved in 20% acetonitrile/water and separated into two 3 mL portions. These portions were purified on a Gilson preparatory HPLC in a gradient of 15%-60% acetonitrile/water (0.1% TFA) over 15 minutes. Two UV absorbance peaks (254 nm) around 10 minutes corresponded to the 3-alpha and 3-beta isomers of the product. The fractions enriched in 3-beta isomer were combined and lyophilized to give ISO-5 (0.183 g, 0.16 mmol, 49%).

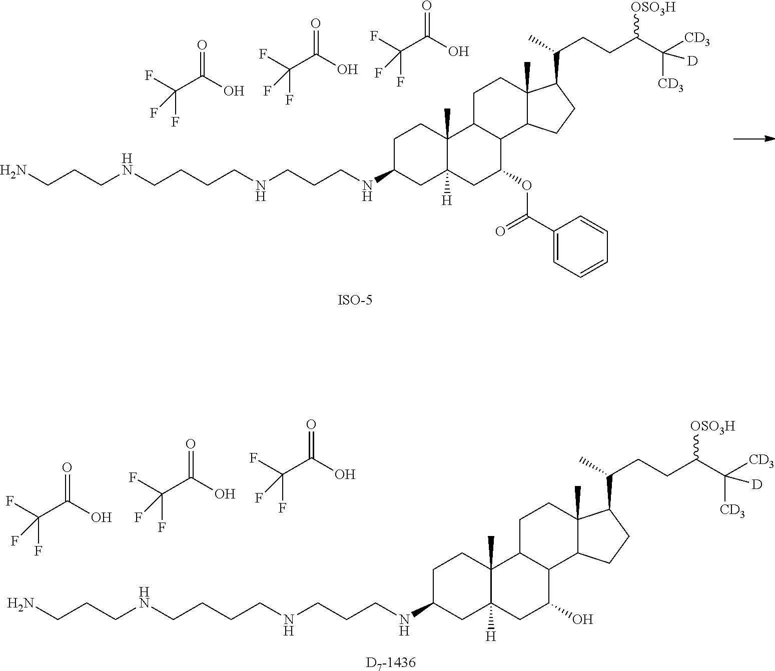

Preparation of D₇-1436.

ISO-5 (0.183 g, 0.16 mmol) was dissolved in 5% methanolic potassium hydroxide solution (25 mL) and refluxed under nitrogen for 72 h, after which TLC (6:3:1 CHCl₃:MeOH:NH₄OH system) indicated that the reaction was complete. Water (15 mL) was added and most of the methanol was evaporated. The solution was acidified with trifluoroacetic acid and applied to a column of AMBERCHROM® in the same procedure as ISO-5. The appropriate fractions were lyophilized to give D₇-1436 (131 mg, 0.13 mmol, 79%) as the tri-TFA salt. ¹H NMR (DMSO-D₆, 300 MHz) δ 4.1 (m, 1H), 3.8 (br s, 1H), 3.2-3.0 (m, 13H), 2.2-1.1 (m, 34H), 1.0 (d, 3H, J=6 Hz), 0.9 (s, 3H), 0.7 (s, 3H); MS (ES−) 690.7 (M−H) (exact: 691.57).

### Example 3b: Preparation of D₇-Squalamine

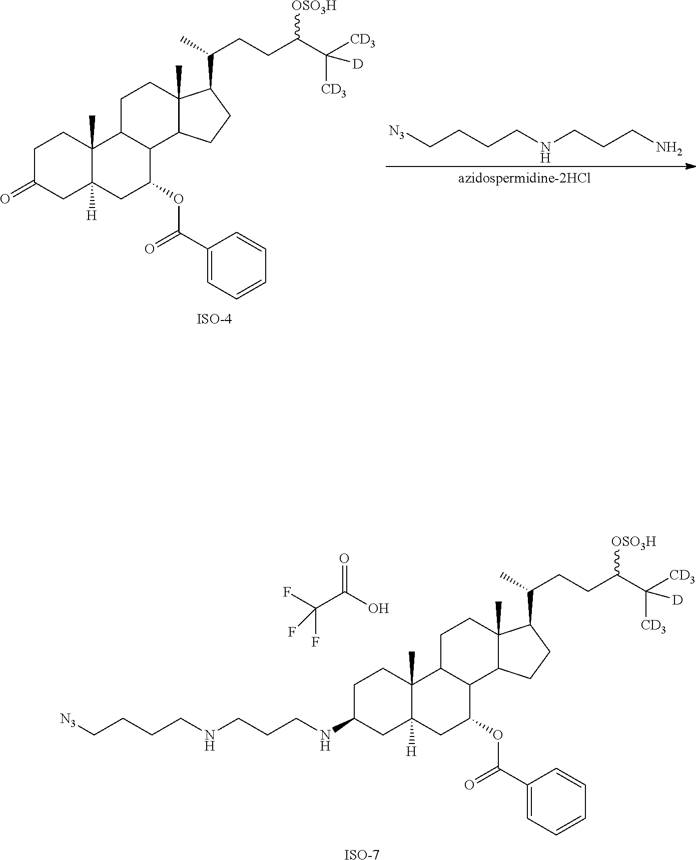

Preparation of ISO-7.

ISO-4 (0.231 g, 0.38 mmol) was dissolved in of 2:1 MeOH/pyridine (20 mL, anhydrous) and treated with 4 equivalents of azidospermidine 2HCl (366 mg), followed by 7.5 equivalents of potassium tert-butoxide. The reaction stirred at room temperature for 2 h, evaporated, again treated with solvent mixture (20 mL), and stirred for another 2 hours. This process was repeated 4 times, with the goal of removing any lingering water through the process of stripping the pyridine from the vessel. After overnight stirring and one final stripping down, 20 mL of 1:1 MeOH/isopropanol was added to the imine residue and brought down to −78° C. in a bath of dry ice and acetone. Five equivalents of NaBH₄ was added in small portions over the course of 30 min, after which time TLC in 6:3:1 with Hannesian stain showed the reaction to be complete. At completion of the reaction, the same procedure was followed as was done for ISO-5 to yield ISO-7 (0.261 g, 0.30 mmol, 78%) after lyophilization.

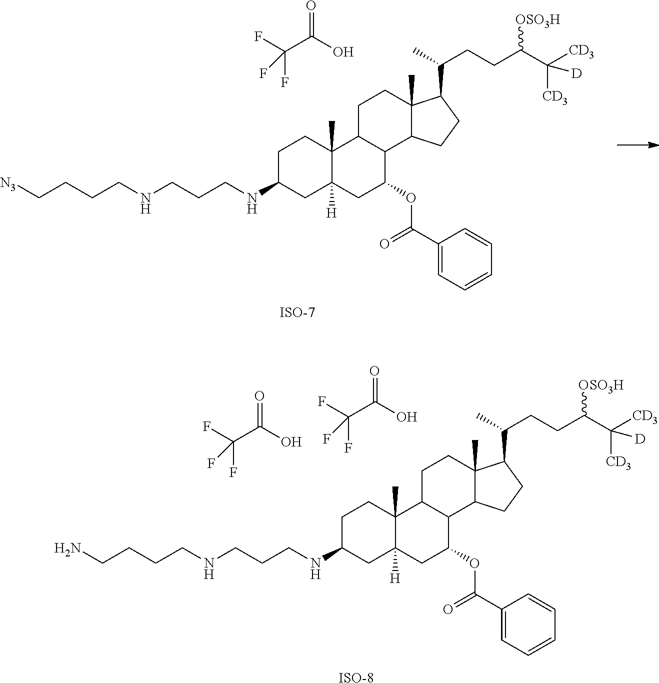

Preparation of ISO-8.

ISO-7 (0.261 g, 0.30 mmol) was dissolved in methanol (50 mL) and charged with Raney Nickel (50 mg). The Parr vessel was placed in a hydrogenator, and shaken overnight under hydrogen (45 psi) at room temperature. The resulting reaction mixture was filtered through CELITE® and evaporated to give ISO-8 (0.245 g). This material was then purified in three portions on the Gilson preparatory HPLC in the same fashion described for compound ISO-5 to afford ISO-8 (142 mg, 0.15 mmol, 50%) as the di-TFA salt.

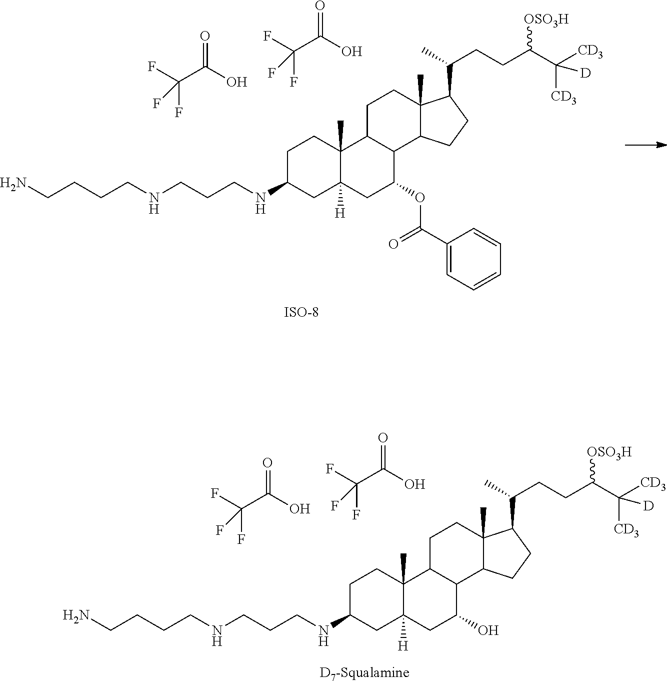

Preparation of D₇-Squalamine.

ISO-8 (0.142 g, 0.12 mmol) was processed in the same manner as ISO-5 to yield D₇-Squalamine (124 mg); however after analysis of the product by HPLC and ¹H NMR, it appeared that not all of the material had hydrolyzed. After some experimentation, it was discovered that pure benzoic acid exhibited virtually the same retention time as D₇-Squalamine, and as a result had co-eluted from the AMBERCHROM® column during the processing of the material after hydrolysis was complete. To separate the benzoic acid impurity, an organic solvent was chosen (diethyl ether) for which benzoic acid is soluble in but for which D₇-Squalamine has little solubility, and so the material was dissolved in water, and extracted 3 times with diethyl ether, after which HPLC confirmed the impurity was no longer present. The aqueous portion was lyophilized to give pure D₇-Squalamine (104 mg, 0.12 mmol, 100%). ¹H NMR (DMSO-D₆, 300 MHz) δ 4.1 (m, 1H), 3.8 (br s, 1H), 3.2-2.9 (m, 9H), 2.2-1.1 (m, 32H), 1.0 (m, 3H), 0.9 (s, 3H), 0.7 (s, 3H); MS (ES+) 635.6 (M+H) (exact mass: 634.51).

### Example 4: Other Sites of Deuterium Incorporation

Deuterium incorporation of squalamine is also performed using the following routes:

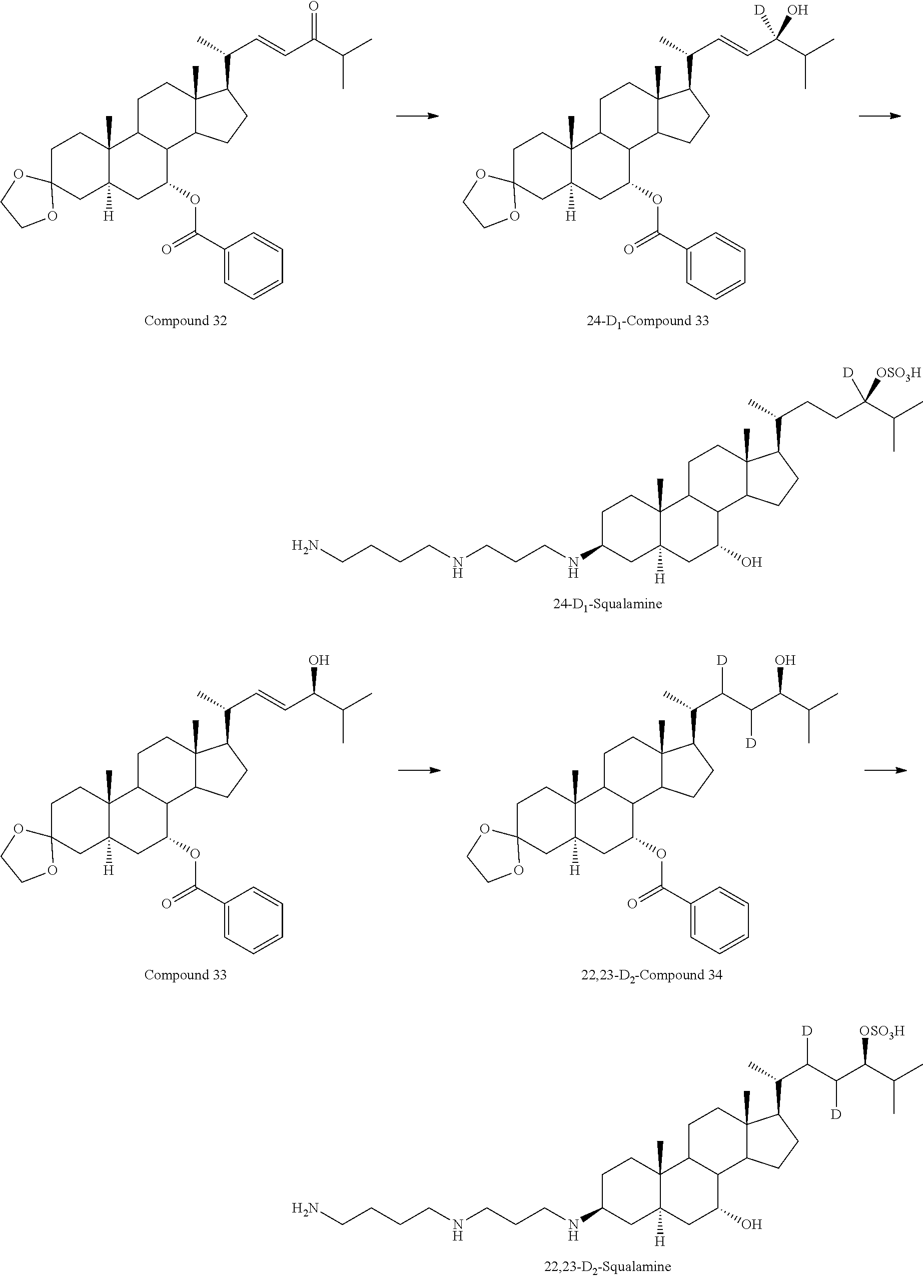

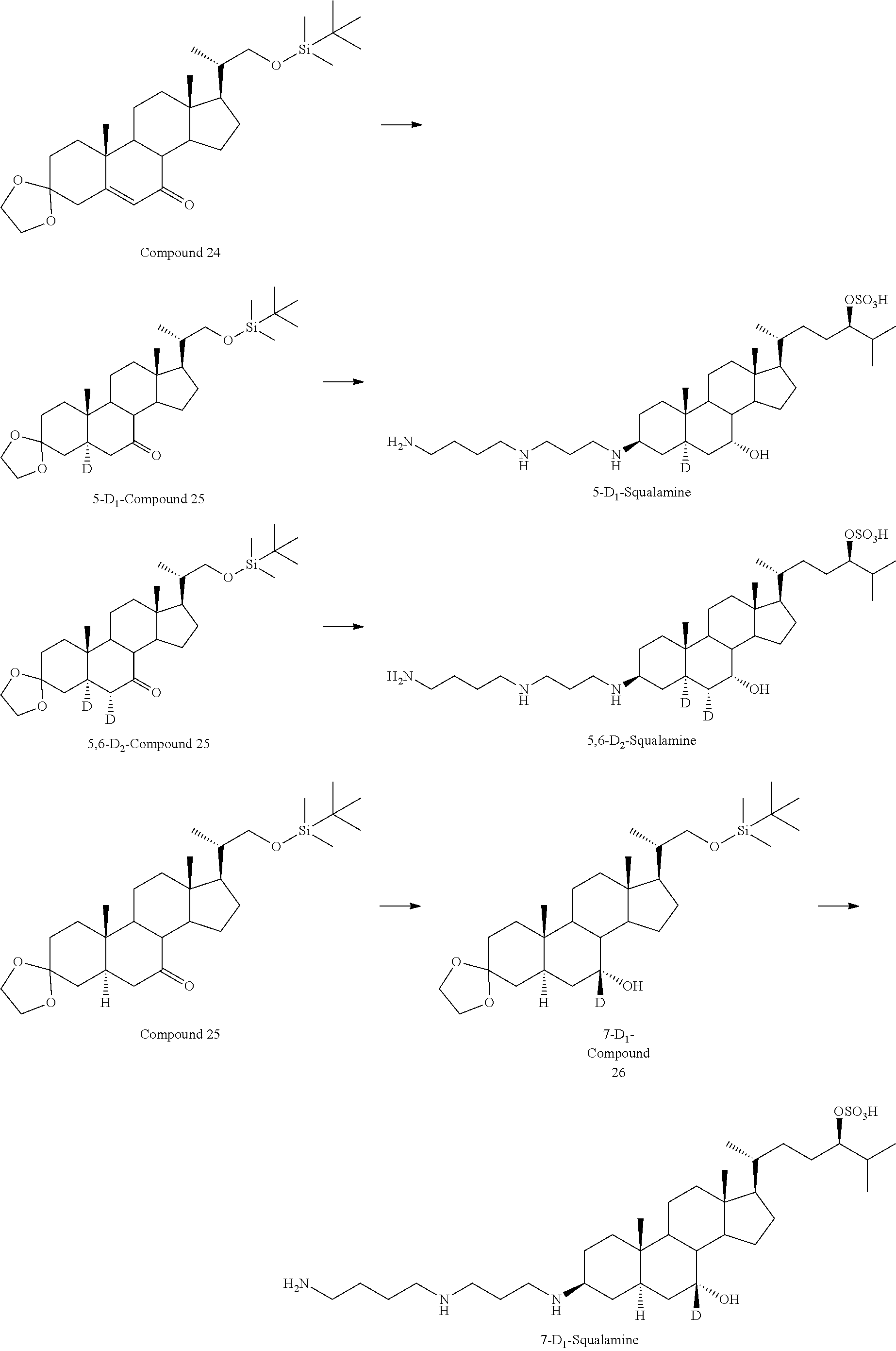

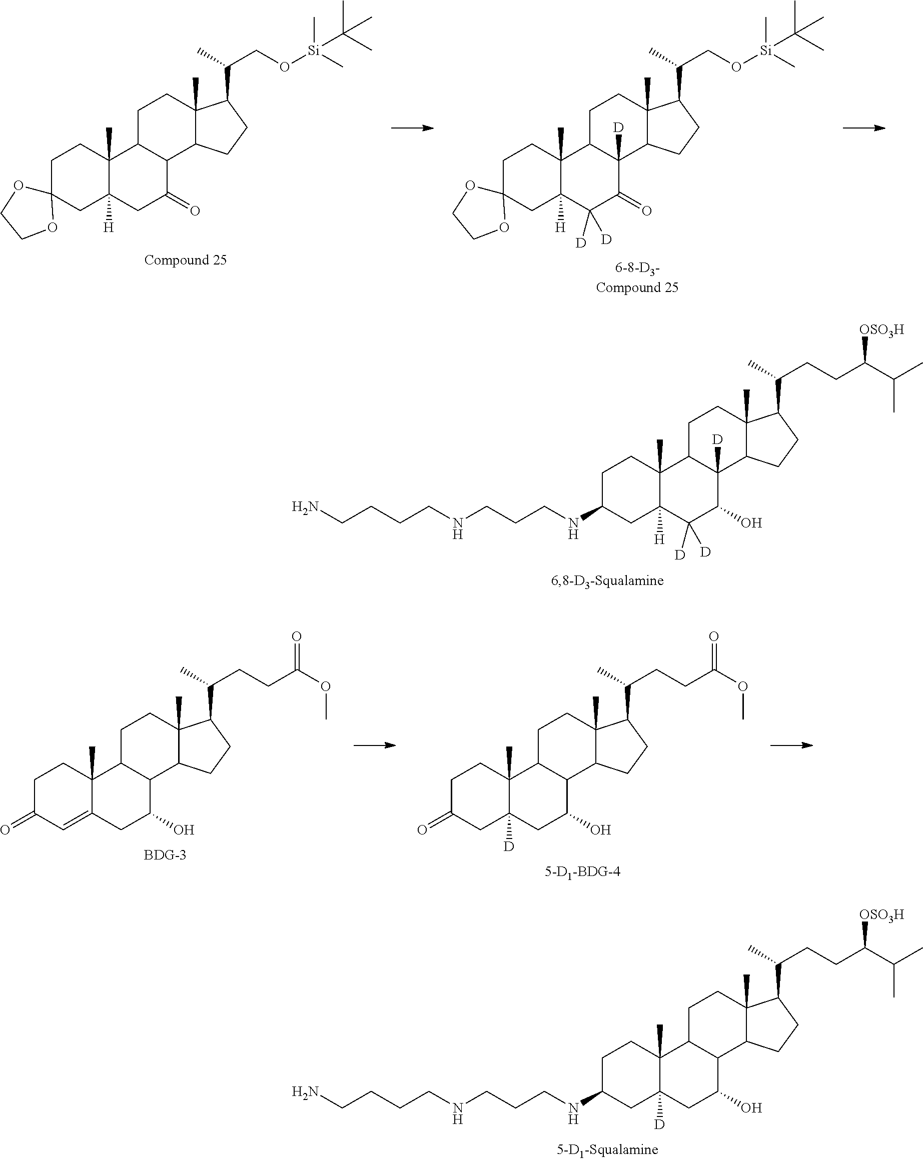

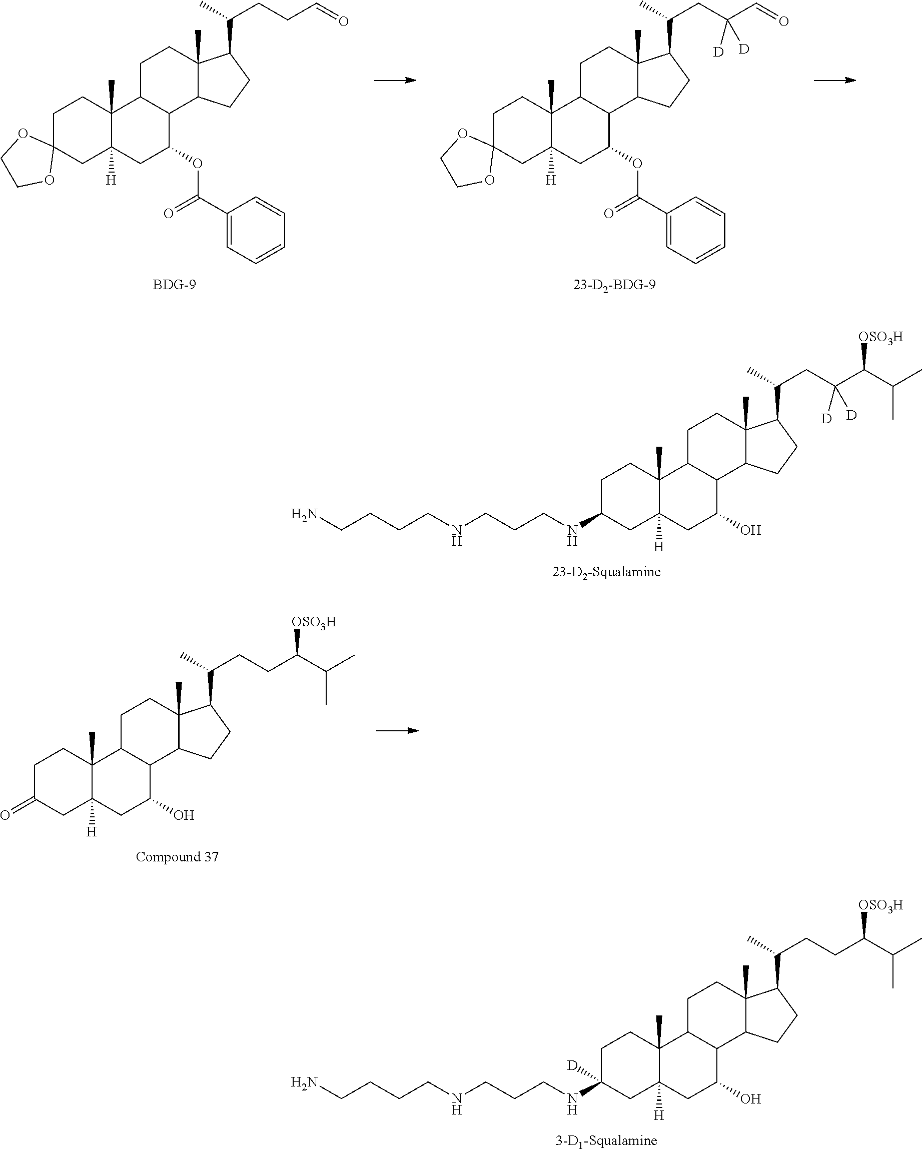

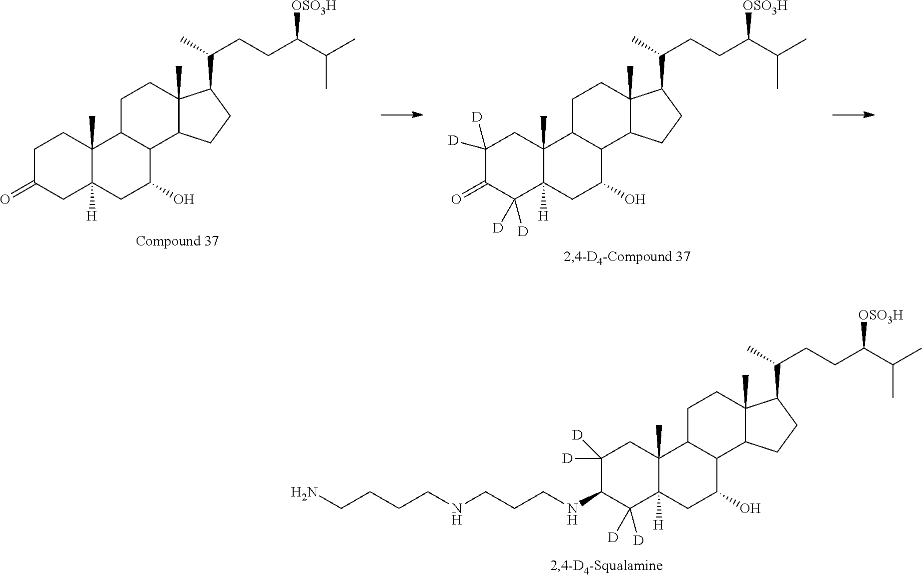

### Example 5. Synthesis of CBZ-Spermidine diHCl

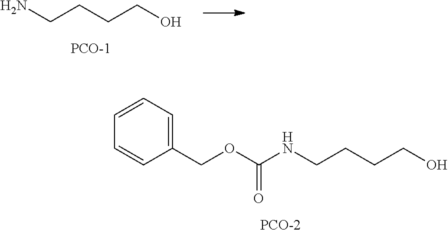

Preparation of PCO-2.

A 1 L reactor was charged with 4-amino-1-butanol (50.0 g, 52.6 mL, 0.561 mol), DCM (500 mL), and TEA (62.4 g, 86.0 mL, 0.617 mol); and cooled to 10° C. Benzylchloroformate (100.5 g, 83.0 mL, 0.589 mol) was added dropwise (30 min) via an addition funnel maintaining the reaction temperature between 10-25° C. The additional funnel was washed with DCM (20 mL) and the reaction was warmed to 20-25° C. When the reaction was complete by HPLC, water (100 mL) was added to the reaction mass to dissolve all solids (triethylamine hydrochloride). The layers were stirred (5 min), settled (15-30 min), and separated. The organic layer was washed with water (3×100 mL), atmospherically distilled to remove DCM (291 mL), maintained at 40° C. with vigorous stirring, and treated dropwise with branched octane (BO, 300 mL) to precipitate product. Crystals were visible after addition of about 150-185 mL of BO (˜½ vol) was added, therefore the addition was paused until the crystallization was complete (resulting in a white slurry). The white slurry was treated with the remaining BO, cooled to 20-25° C., filtered, washed twice with BO (2×200 mL), and dried under vacuum overnight (50° C.) to yield PCO-2 (85.18 g, 68.0%). Additional pure material was obtained from the mother liquors at rt (2.63 g, 2.1%) and 0° C. (5.3 g, 4.2%). ¹H NMR (400 MHz, CDCl₃) δ ppm 1.57 (m, 4H) 3.20 (m, 2H) 3.63 (m, 2H) 5.05 (s, 2H) 7.34 (m, 5H); ¹³C NMR (100 MHz, CDCl₃) δ ppm 26.37, 29.49, 40.80, 62.11, 66.55, 127.93, 127.98, 128.40, 136.50, 156.55; MS (ESI) 224.1 (M+H).

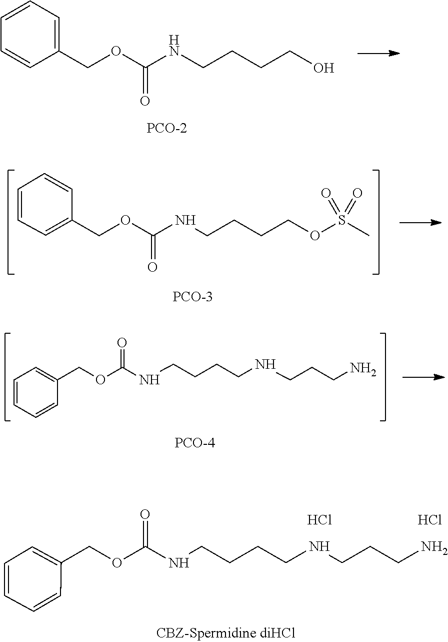

Synthesis of CBZ-Spermidine diHCl.

A 2 L Reactor was purged with alternating vacuum and nitrogen (3×) cycles, charged with PCO-2 (100 g, 0.448 mol), methyltetrahydrofuran (MeTHF, 1,000 mL), and triethylamine (52.1 g, 71.8 mL, 0.515 mol). The reaction mixture was warmed (15-20° C.) to dissolve all solids and treated via addition funnel with methane sulfonyl chloride (59.0 g, 39.9 mL, 0.515 mol) dropwise (28 min) maintaining the reaction temperature below 30° C. MeTHF (20 mL) was added to addition funnel and transferred to the reactor, which was continued for 30 min at 20-30° C. at which point no starting material remained by HPLC. Water (300 mL) was added to dissolve the solids, and the mixture was stirred, settled and separated. The organic layer was washed with water (300 mL), distilled under vacuum (distillate=740 mL, T_{d} is =18° C.) to about 250 mL volume, and transferred to an addition funnel (50 mL MeTHF rinse) providing PCO-3. The reactor was charged with 1,3-diaminopropane (DAP, 1,866 mL), heated to 45° C., and treated dropwise (30-45 min) with the solution of PCO-3, maintaining reaction at 35-45° C. The reaction mixture was a clear colorless solution throughout addition. The MeTHF was distilled off under house vacuum (Tₚₒₜ 51.8-74.4° C.), followed by DAP (Tₚₒₜ=93.7° C., 1,415 g of DAP recovered), leaving about 230 mL of solution in reactor, which was held at 20° C. overnight. DCM (600 mL) was added to the reaction mass and stirred to dissolve. A solution of sodium hydroxide (20 g) in water (200 mL) was added, and the layers were stirred, settled (1 h, 21° C.), and separated. The DCM layer was washed with water (150 mL) at 0-5° C. to obtain a good separation. Then an additional water (150 mL) and saturated sodium chloride solution (50 mL) were add at low temperature and the layers stirred, settled and separated. Finally, saturated sodium chloride (100 mE) was added and the layers stirred, settle and separated (2×). The combined aqueous phases were extracted with DCM (2×100 mL) and combined with other DCM solution. The organic layer containing PCO-4 was concentrated by atmospheric distillation to about 250 mL, methanol (250 mL) was added, and then the solvent distilled. The methanol exchange was repeated two more times and then methanol (250 mL) was added and the solution (500 mL) was cooled to 0-5° C. Methanol (300 mL) was added to a separate flask, cooled to less than 5° C., and treated with acetyl chloride (96 mL, 3 eq) slowly to form an anhydrous HCl solution, which was then added to the Cbz-spermidine solution at 0-5° C. with stirring. The product was filtered, washed with 0-5° C. methanol (2×200 mL), and dried in a vacuum oven at 50° C. overnight to afford CBZ-Spermidine diHCl (120.97 g, 76.7%). To enhance purity, CBZ-Spermidine diHCl (100 g) was recrystallized in a nitrogen purged reactor equipped with a condenser, thermocouple, mechanical agitator and nitrogen inlet. Methanol (1,000 mL) and water (100 mL) were added and the reaction mixture was heated to reflux and stirred until dissolved. Crystallization was achieved by slowly lowering temperature of jacket from 70-50° C. over 120 min with a cloud point around 57-59° C. (t=60-70 min). The product crystallized to form a nice white slurry as it cooled to 50° C. The slurry was further cooled to 0-5° C. over 1 h, filtered at 0-5° C., washed with cold (0-5° C.) methanol (2×200 mL), and dried under vacuum at 50° C. to constant weight (77.5 g). MS (ESI) 280 (M+H); ¹H NMR (DMSO-D₆, 600 MHz) δ 9.12 (br s, 2H), 8.19 (br s, 3H), 7.37-7.31 (m, 5H), 5.01 (s, 2H), 3.02 (m, 2H), 2.96 (m, 2H), 2.90 (m, 2H), 2.83 (m, 2H), 1.98 (m, 2H), 1.63 (m, 2H), 1.46 (m, 2H); ¹³C NMR (DMSO-D₆, 150 MHz) δ 156.07, 137.15, 128.28, 127.64, 65.10, 46.39, 43.68, 36.02, 26.45, 23.54, 22.69.

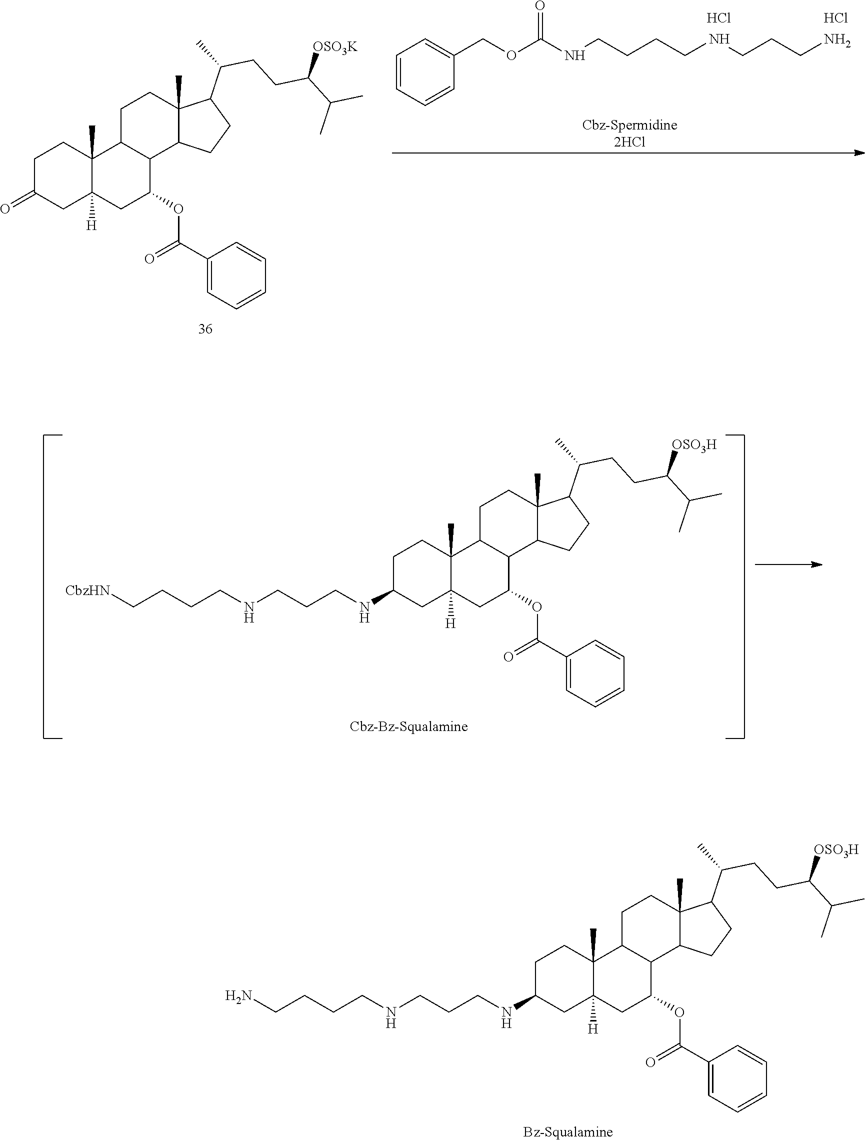

Synthesis of Bz-Squalamine.

CBZ-spermidine diHCl (6.87 g, 19.5 mmol) and MeOH (400 mL) were added to a 1 L flask and treated with 25% sodium methoxide (10.7 mL, 10.22 g, 46.8 mmol) solution in methanol in one portion, after which clarity was achieved. Compound 36 was added and the reaction mixture was stirred at rt overnight. The following day, the reaction mixture was cooled in a dry ice/acetone bath for 30 min and treated with sodium borohydride three portions (1.00 g initially, 0.4 g 15 min later, 0.4 g 15 min later; 1.77 g, 46.8 mmol). The reaction mixture was stirred in the dry ice bath as the dry ice was allowed to evaporate in the hood for 1 h. Water (50 mL) was added and the reaction mixture was stirred for 5 min, placed on the rotary evaporator, and evaporated to remove methanol. Water (50 mL) and 2-BuOH (100 mL) were added to the residue, the biphasic mixture was stirred for 5 min, and then allowed to separate. The aqueous phase and interface were extracted with 2-BuOH (50 mL). The organic phases were combined, washed with a mixture of water (35 mL) and brine (15 mL), and then evaporated to a residue (Bath temp: 50° C.). MeOH (100 mL) was added and the reaction mixture stirred at rt for 5 min (some undissolved solids present), heated with a heat gun briefly (still some minor undissolved solids were present), cooled to rt, and stirred for 30 min (observed to be a clear solution). Palladium on Carbon (3.32 g, 1.56 mmol, 0.1 equiv., 50% water wet, 10% Pd/C) was added to the reaction mixture followed by formic acid (2.94 mL, 78 mmol, 5.0 equiv.). The reaction mixture heated to 50° C. and HPLC analysis after 1 h showed the reaction to be complete. The reaction mixture was allowed to cool to rt, stirred overnight, filtered through a CELITE® pad, rinsed with methanol, evaporated, and treated with methanol (50 mL) to yield a clear solution. Separately, acetyl chloride (3.33 mL, 46.8 mmol, 3.0 equiv.) was added to cold methanol (50 mL) to generate an anhydrous hydrogen chloride solution, which was then added dropwise to the reaction mixture over 1 h in an ice bath, resulting in a slurry. The reaction mixture was stirred at rt for 2 h, treated with methyl t-butyl ether (MTBE, 50 mL), and stirred overnight. Because filtration was poor, the reaction mixture was evaporated to a residue on a rotary evaporator, treated with MTBE (100 mL) and it was stirred at rt for 30 min. The reaction mixture was again evaporated, treated with MTBE (100 mL) and stirred at rt for 30 min. The solids were collected by filtration (moderate filtration speed—5 min), rinsed with MTBE (50 mL), slurried with MTBE (50 mL) on filter funnel with a spatula, and then filtered. The solids were dried in a vacuum oven at 50° C. until constant weight was achieved. Bz-Squalamine trihydrochloride was isolated as a white solid (12.57 g, HPLC: 67% overall purity at 230 nm; 2.7% 3-alpha; 96/4 beta:alpha ratio), MS (ES+) 732.5 (M+H) (exact mass: 731.5). Bz-Squalamine is a useful intermediate in the preparation of squalamine. The same chemistry is useful in the preparation of deutero-analogs of squalamine.

### Example 6. Activity for D₇-1436 as an Inhibitor of Protein Tyrosine Phosphatase 1B (PTP1B)

MSI-1436 and D₇-1436 (D-1436) were dissolved in DMSO to a stock concentration of 10 mM. A known PTP1B inhibitor served as a control. The compounds were tested in a 10-dose IC₅₀ mode with 3-fold serial dilution, in singlet, starting at 100 μM. The enzyme was the human truncated form (1-321), recombinantly produced in E. coli. Fluorescence was measured to monitor enzyme activity. As seen in Table 1, MSI-1436, already known to inhibit PTP1B, exhibited an IC₅₀ of 2.89 μM; D₇-1436 exhibited an IC₅₀ of 2.09 μM; and the control PTP1B compound exhibited an IC₅₀ of 2.47 μM. These data demonstrate that D₇-1436 inhibits PTP1B.

TABLE 1
Inhibition of PTP1B by MSI-1436 and D7-1436

| | | | | |
|---|---|---|---|---|
| | | | | PTP1B |
| Conc (M) | MSI-1436 | D-1436 | Conc (M) | Inhibitor |

Slope (signal/min)

| | | | | |
|---|---|---|---|---|
| 1.00E−04 | −68 | −161 | 1.00E−04 | −810 |
| 3.33E−05 | −440 | −362 | 3.33E−05 | −444 |
| 1.11E−05 | 292 | −319 | 1.11E−05 | 1291 |
| 3.70E−06 | 16431 | 8429 | 3.70E−06 | 18367 |
| 1.23E−06 | 37260 | 34760 | 1.23E−06 | 34136 |
| 4.12E−07 | 39959 | 41755 | 4.12E−07 | 40760 |
| 1.37E−07 | 40137 | 40150 | 1.37E−07 | 42024 |
| 4.57E−08 | 42717 | 43697 | 4.57E−08 | 45466 |
| 1.52E−08 | 44021 | 43819 | 1.52E−08 | 47019 |
| 5.08E−09 | 43513 | 44655 | 5.08E−09 | 48684 |
| DMSO | 47697 | 47400 | DMSO | 47769 |

% Activity

| | | | | |
|---|---|---|---|---|
| 1.00E−04 | −0.16 | −0.37 | 1.00E−04 | −1.88 |
| 3.33E−05 | −1.02 | −0.84 | 3.33E−05 | −1.03 |
| 1.11E−05 | 0.68 | −0.74 | 1.11E−05 | 2.99 |
| 3.70E−06 | 38.12 | 19.55 | 3.70E−06 | 42.61 |
| 1.23E−06 | 86.43 | 80.63 | 1.23E−06 | 79.19 |
| 4.12E−07 | 92.69 | 96.86 | 4.12E−07 | 94.55 |
| 1.37E−07 | 93.11 | 93.14 | 1.37E−07 | 97.48 |
| 4.57E−08 | 99.09 | 101.36 | 4.57E−08 | 105.47 |
| 1.52E−08 | 102.11 | 101.65 | 1.52E−08 | 109.07 |
| 5.08E−09 | 100.94 | 103.59 | 5.08E−09 | 112.93 |
| DMSO | 110.64 | 109.95 | DMSO | 110.81 |
| HILLSLOPE | −2.19 | −2.50 | HILLSLOPE | −1.47 |
| IC50 (M) | 2.89E−06 | 2.09E−06 | IC50 (M) | 2.47E−06 |

### Example 7. Pharmacological Activity for D₇-1436 in Mice as Weight Loss Agent

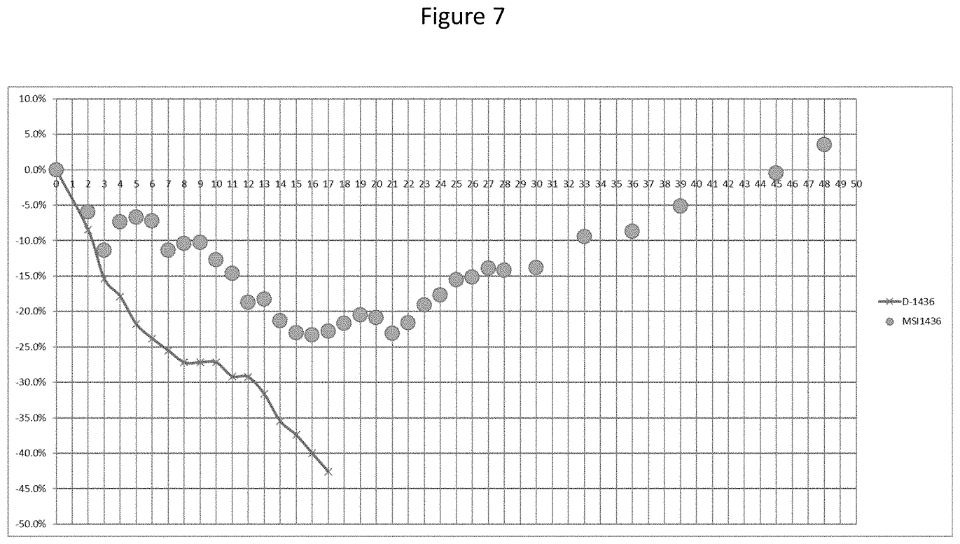

MSI-1436 is known to induce weight loss through a mechanism that involves certain brain circuits that control appetite. To determine if D₇-1436 (D-1436) exhibited similar activity, male Swiss Webster mice (about 50 grams, N=3 for each group) were treated i.p. with either MSI-1436 or D₇-1436 at 10 mg/kg on days 0, 2, 4, 6, 8 and 10. Food was provided ad lib. As seen in FIG. 7, administration of D₇-1436 resulted in a continuous decline in weight, eventually reaching about 45% of the starting weight by day 16. The animals appeared moribund and were euthanized. In contrast, animals treated with MSI-1436 reached a nadir in weight around day 16 and then began a gradual recovery, reaching their starting weight by day 45.

It was hypothesized that deuterium atoms at positions C25, C26 and C27 might slow the rate of compound metabolism due to an isotope effect. The results surprisingly showed increased potency of D₇-1436 as compared with MSI-1436. These data demonstrate that isotope substitution within the cholestane side chain can dramatically increase the pharmacological activity of MSI-1436 and other aminosterols such as squalamine.

### Example 8

This example describes an exemplary method of treating and/or preventing symptoms of Parkinson's disease (PD) in a clinical trial setting using an undeuterated aminosterol, squalamine phosphate (ENT-01). While the example describes data relating to undeuterated squalamine, the data described herein is predictive of success likely to be obtained with a deuterated squalamine, as deuteration is not known to change the biological activity of a compound.

Overview:

The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-it and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squalamine (ENT-01; Enterin, Inc.) was formulated for oral administration in the trial. The active ion of ENT-01, squalamine, an aminosterol originally isolated from the dogfish shark, has been shown to reverse gastrointestinal dysmotility in several mouse models of PD. In addition, ENT-01 has been shown to inhibit the formation of aggregates of αS both in vitro, and in a C. elegans model of PD in vivo (Perni et al. 2017). In the C. elegans model, squalamine produced a complete reversal of muscle paralysis.

ENT-01 is the phosphate salt of squalamine. For this study it has been formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz water. The dose was taken by each patient upon awakening on an empty stomach along with 8 oz. of water simultaneously to dopamine. The subject was not allowed to ingest any food for at least 60 minutes after study medication. The compound is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m²).

In the current clinical trial, squalamine (ENT-01) was administered orally to subjects with PD who have long standing constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg (14.7 mg/m²) to 250 mg (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night.);

(6) I-Button Temperature Assessment. The I-Button is a small, rugged self-sufficient system that measures temperature and records the results in a protected memory section. The Thermochron I-Button DS 1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated.);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and phychosis, 1.3 depressed mood, Part II=Motor Aspects of Experiences of Daily Living (M-EDL), Part III=Motor Examination, and Part IV=Motor Complications;

(10) Mini Mental State Examination (MMSE);

(11) Trail Making Test (TMT) Parts A and B;

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al. 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS 1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with PD and constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design: A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al. 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours have elapsed, and provided there are no safety concerns, the patient was sent home and brought back on day 4-8 for the next dose. During the days the subjects are home, they completed the daily diaries and e-mailed them to the study coordinators. Subjects 3-10 were dosed after the first 2 subjects have been observed for 72 hours, i.e. on Day 4. Subjects 1-2 were also brought back on Day 4-8 and given a single dose of 50 mg. Once another 24 hours have elapsed and provided there are no safety concerns, the patients were all sent home and instructed to return on Day 7 for the next dosing level. This single dosing regimen was continued until each subject was given a single dose of 200 mg or has reached a dose limiting toxicity (DLT). DLT was the dose which induces repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al. 1987). Patients were required to have a history of constipation as defined by <3 CSBMs/week and satisfy the Rome IV criteria for functional constipation (Mearin et al. 2016) at screening, which requires 2 or more of the following: Straining during at least 25% of defecations; lumpy or hard stools in at least 25% of defecations; sensation of incomplete evacuation in at least 25% of defecations; sensation of anorectal obstruction/blockage in at least 25% of defecations; and/or manual maneuvers to facilitate at least 25% of defecations.

Baseline characteristics of patients are shown in Table 1. Patients in Stage 2 had somewhat longer duration of Parkinson's disease and higher UPDRS scores than participants in Stage 1.

TABLE 1
Baseline Characteristics of Dosed Patients

| | | | |
|---|---|---|---|
| | Stage 1** | Stage 2*** | Total |
| Characteristic | (n = 10) | (n = 34) | (n = 44) |

Sex- no. (%)

| | | | |
|---|---|---|---|
| Male | 5 (50) | 25 (73.5) | 30 (68.1) |
| Female | 5 (50) | 9 (26.5) | 14 (31.8) |
| White race-no. (%) | 8 (80) | 34 (100) | 42 (95.54) |

Age-yr

| | | | |
|---|---|---|---|
| Mean | 65.0 | 74.5 | 72.5 |
| Range | 58-70.5 | 60.6-84.2 | 58-84.2 |

Age at PD diagnosis-yr

| | | | |
|---|---|---|---|
| Mean | 61.1 | 67.7 | 66.2 |
| Range | 54.2-69 | 50.6-82.5 | 50.6-82.5 |

Duration of PD-yr

| | | | |
|---|---|---|---|
| Mean | 4.2 | 6.8 | 6.2 |
| Range | 1-11 | 0.3-17.3 | 0.3-17.3 |

Duration of constipation-yr

| | | | |
|---|---|---|---|
| Mean | 25.8 | 16.8 | 18.9 |
| Range | 1-65 | 0.5-66.0 | 0.5-66.0 |

UPDRS score

| | | | |
|---|---|---|---|
| Mean | 53.4 | 63.2 | 61.3 |
| Range | 33-88 | 24-122 | 24.0-122.0 |

Hoehn and Yahr-Stage

| | | | |
|---|---|---|---|
| Mean | 2.0 | 2.4 | 2.3 |
| Range | 2.0 | 1.0-5.0 | 1.0-5.0 |

Constipation severity* - CSBM/wk- no. (%)

| | | | |
|---|---|---|---|
| 0-1 | 8 (80) | 14 (41.2) | 22 (50) |
| 1.1-2 | 2 (20) | 17 (50) | 19 (43.2) |
| 2.1-3 | 0 | 3 (8.8) | 3 (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

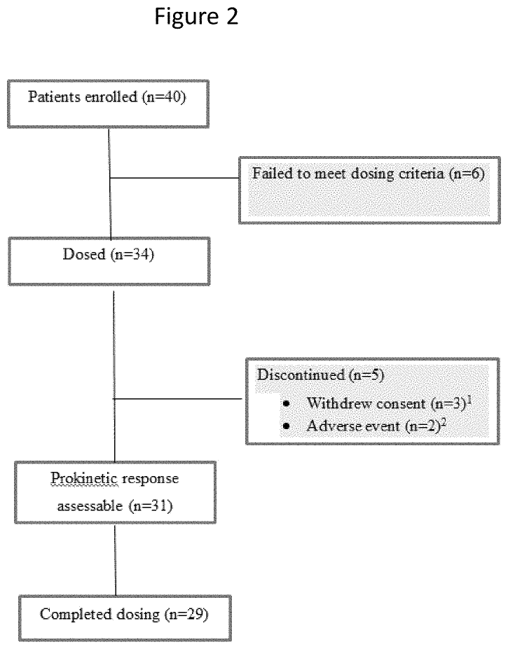

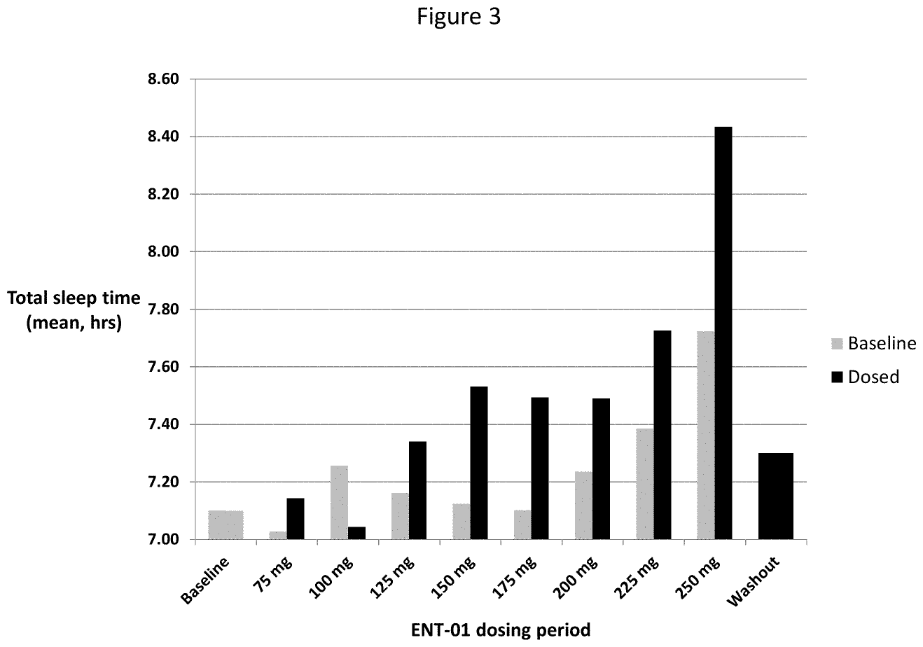

Safety and Adverse Event (AE) Profile: Fifty patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 2 and FIG. 2.

TABLE 2
Study drug assignments and adherence to treatment

| | Stage 1 | Stage 2 | |
|---|---|---|---|
| Enrolled | 10 | 40 | |
| Failed prior to dosing | 0 | 6 | |
| Dosed | 10 | 34 | |
| 25-200 mg | 10 | | |
| 75-175 mg | | 19 | |
| 100-250 mg | | 15 | |
| Terminated (%) | 0 (0) | 2* | (5.8) |
| Withdrew (%) | 1 (10) | 3 | (8.8) |
| Completed dosing (%) | 9 (90) | 31** | (91) |

| | |
|---|---|
| Randomized | 15 |
| Treatment | 6 |
| Placebo | 9 |

The 2 patients who were terminated
**29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2. Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 3.

TABLE 3
All adverse events (n, %)

Enrolled

| | | |
|---|---|---|
| | Stage 1 (n = 10) | Stage 2 (n = 40) |
| Dosed | 10 | 34 |

GI:
Nausea

| | | |
|---|---|---|
| Mild | 4(40) | 18(52) |
| Moderate | 0 | 1(2.9) |

Diarrhea

| | | |
|---|---|---|
| Mild | 1(10) | 12(35) |
| Moderate | 3(30) | 2(5.8) |
| Severe | 0 | 1(2.9) |

Vomiting

| | | |
|---|---|---|
| Mild | 1(10) | 2(5.8) |
| Moderate | 0 | 0 |

Abdominal pain

| | | |
|---|---|---|
| Mild | 2(20) | 4(11.7) |
| Moderate | 3(30) | 2(5.8) |

Flatulence

| | | |
|---|---|---|
| Mild | 2(20) | 1(3) |
| Moderate | 0 | 0 |

Loss of appetite*

| | | |
|---|---|---|
| Mild | 1(10) | 0 |
| Moderate | 0 | 0 |

Worsening acid reflux

| | | |
|---|---|---|
| Mild | 0 | 4(11.7) |
| Moderate | 0 | 0 |

Worsening hemorrhoid

| | | |
|---|---|---|
| Mild | 0 | 1(3) |
| Moderate | 0 | 0 |

Lower GI bleed**

| | | |
|---|---|---|
| Severe | 0 | 1(2.5) |

Non-GI:
Dizziness

| | | |
|---|---|---|
| Mild | 0 | 7(20.5) |
| Moderate | 0 | 1(2.9) |

Blood in urine*

| | | |
|---|---|---|
| Mild | 1(10) | 0 |
| Moderate | 0 | 0 |

Headache

| | | |
|---|---|---|
| Mild | 1(10) | 3(8.8) |
| Moderate | 0 | 0 |

Urinary retention

| | | |
|---|---|---|
| Mild | 0 | 1(3) |
| Moderate | 0 | 0 |

Urinary tract infection

| | | |
|---|---|---|
| Dosed | 10 | 34 |
| Mild | 0 | 1(3) |
| Moderate | 0 | 2(5.8) |

Increased urinary frequency

| | | |
|---|---|---|
| Mild | 0 | 2(5.8) |
| Moderate | 0 | 0 |

Skin lesions-rash

| | | |
|---|---|---|
| Mild | 0 | 3(8.8) |
| Moderate | 0 | 0 |

Eye infection

| | | |
|---|---|---|
| Mild | 0 | 1(3) |
| Moderate | 0 | 0 |

Difficulty falling asleep

| | | |
|---|---|---|
| Mild | 0 | 1(3) |
| Moderate | 0 | 0 |

*Unrelated to ENT-01
**colonic diverticulosis, polyp, patient on aspirin, Plavix and naproxen. Unrelated to ENT-01

TABLE 4
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |
| 250 | — | — | — | 2 | — | |

*lightheadedness included

TABLE 5
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural | Moderately symptomatic and |
| hypotension | limiting daily activities or BP < 80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related”. The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

Cumulative responder rates of bowel function are shown in FIG. 1A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10-8) and SBM increased from 2.6 at baseline to 4.5 at fixed dose (p=6.4×10-6) (Table 6). Use of rescue medication decreased from 1.8/week at baseline to 0.3 at fixed dose (p=1.33×10-5). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 6
Stool related indices Stage 2 (Dosed patients, n = 34)

| | Baseline | Fixed dose | |
|---|---|---|---|
| | (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1-manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 1B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 7).

TABLE 7
Reversal of stool indices to baseline
during the wash-out period (Stage 2)

| | | | | P-value |
|---|---|---|---|---|
| | Baseline | Fixed dose | Wash-out | (wash-out vs. |
| | (Mean, SD) | (Mean, SD) | (Mean, SD) | baseline) |
| CSBM | 1.2 (0.90) | 3.8 (2.4) | 1.8 (1.19) | 0.01 |
| SBM | 2.6 (1.45) | 4.5 (2.21) | 3.2 (1.80) | 0.16 |
| Ease | 3.2 (0.73) | 3.7 (1.19) | 3.3 (0.81) | 0.78 |
| Consistency | 2.7 (1.20) | 4.1 (2.13) | 2.8 (1.39) | 0.85 |
| Rescue meds | 1.8 (1.92) | 0.3 (0.67) | 1.0 (1.40) | 0.13 |
| PAQ-QOL | 1.4 (0.49) | 1.2 (0.59 | 1.2 (0.63) | 0.04 |
| PAQ-SYM | 1.3 (0.45) | 1.1 (0.49) | 1.1 (0.60) | 0.11 |

The primary efficacy outcome variable was whether or not a subject was a “success” or “failure”. This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure”. The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 8).

TABLE 8
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmakokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 9). In Stage 2, PK was measured on days 1 and 6 of the randomization period pre-medication, at 1, 2, 4 and 8 hours (Table 10). Based on the pharmacokinetic behavior of intravenously administered squalamine determined in prior clinical studies it is estimated that squalamine (ENT-01) exhibited oral bio-availability of less than 0.3% (Bhargava et al. 2001; Hao et al. 2003).

TABLE 9
Pharmacokinetics of orally administered squalamine (ENT-01) in Stage 1.
Stage 1

| | | | Tₘₐₓ | | | |
|---|---|---|---|---|---|---|
| | | | (hour) | T_{1/2} | | |
| Dose | # of | Cₘₐₓ | (Median | (hours) | AUC_{0-8 hr} | AUC_{0-16 hr} |
| (mg) | patients | (ng/ml) | Value) | (n) | (ng*hour/ml | (ng*hour/ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9.0 (5) | 78.3 | 157 |

TABLE 10
Pharmacokinetics of orally administered
squalamine (ENT-01) in Stage 2.
Stage 2

| | # of | | | | |
|---|---|---|---|---|---|
| | patients | | Tₘₐₓ (hour) | T_{1/2} | |
| Dose | (2 visits | Cₘₐₓ | (Median | (hours) | AUC_{0-8 hr} |
| (mg) | each) | (ng/ml) | Value) | (n) | (ng*hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ and T_{1/2} and AUC of the squalamine ion following squalamine (ENT-01) oral dosing for Stage 1 patients. The PK analyses are only approximate, as the lower limit of the validated concentration range was 10 ng/ml; most of the measured concentrations fell below that value. The mean Cₘₐₓ, Tₘₐₓ and T_{1/2} and AUC of the squalamine ion following squalamine (ENT-01) oral dosing for Stage 2 patients. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS symptoms: CNS symptoms were evaluated at baseline and at the end of the fixed dose period and the wash-out period (Table 11). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MMSE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by 5 patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4). Unlike stool-related indices, the improvement in many CNS symptoms persisted during wash-out.

TABLE 11
Effect of Squalamine (ENT-01) on neurological symptoms (n = 34)

| | Baseline | Fixed dose | | Wash-out | |
|---|---|---|---|---|---|
| UPDRS | (Mean, SD) | (Mean, SD) | P-value | (Mean, SD) | P-value |
| Part 1 | 11.6 (6.51) | 10.6 (6.18)) | 0.28 | 9.5 (5.27) | 0.06 |
| (NMS) | | | | | |
| Part 2 | 14.9 (8.11) | 14.7 (9.02) | 0.77 | 14.1 (8.21) | 0.40 |
| (Daily | | | | | |
| living) | | | | | |
| Part 3 | 35.3 (14.35) | 33.3 (15.20) | 0.13 | 30.2 (13.23) | 0.005 |
| (Motor) | | | | | |
| Total | 64.4 (23.72) | 60.6 (25.60) | 0.09 | 55.7 (23.69) | 0.002 |
| MMSE | 28.4 (1.75) | 28.7 (1.9) | 0.21 | 29.3 (1.06) | 0.0006 |
| PDHQ | 1.3 (2.99) | 1.8 (3.34) | 0.45 | 0.9 (2.33) | 0.03 |
| BDI-II | 10.9 (7.12) | 9.9 (6.45) | 0.14 | 8.7 (5.19) | 0.10 |

UPDRS: Unified Parkinson's Disease Severity Score;
NMS: Non-motor symptoms;
BDI: Beck Depression Index-II;
MMSE: Mini-mental State exam.
PDHQ: Parkinson's Disease Hallucination Questionnaire

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS 1921H; Maxim, Dallas, Tex.) (Sarabia et al. 2008). A nonparametric analysis was performed for each participant to characterize DST as previously described (Sarabia et al. 2008; Ortiz-Tudela et al. 2010).

Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm (Ortiz-Tudela et al. 2010).

A comparison was performed of circadian rhythm parameters during the baseline, fixed dose and washout periods. ENT-01 administration improved all markers of healthy circadian function, increasing rhythm stability (IS, p=0.026), relative amplitude (RA, p=0.001) and circadian function index (CFI, p=0.016), while reducing rhythm fragmentation (IV, p=0.031). The improvement persisted for several of these circadian parameters during wash-out period (IS, p=0.008 and CFI, p=0.004). (FIG. 5).

Conclusions:

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamic responses of ENT-01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing tS and stimulating enteric neurons. These results demonstrate that the ENS in PD is not irreversibly damaged and can be restored to normal function.

Several exploratory endpoints were incorporated into the trial to evaluate the impact of ENT-01 on neurologic symptoms associated with PD. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 11).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose at 175 mg. The patient remained free of hallucinations for 1 month following cessation of dosing. RBD and total sleep time also improved progressively in a dose-dependent manner.

The prokinetic effect of the aminosterol squalamine appears to occur through local action of the compound on the ENS, since squalamine, the active zwitterion, is not significantly absorbed into the systemic circulation.

### Example 9—Constipation

This prophetic example describes an exemplary method of (i) treating constipation and/or (ii) treating and/or preventing a disorder in which constipation is a known symptom (e.g., a constipation associated disorder) in a subject. The method comprises administering a deuterated aminosterol to a subject in need.

Patients are selected based on the constipation criteria described in Example 8. Patients are grouped based on having a particular constipation associated disorder or having constipation with no underlying disorder. The groups are then subdivided into a control subgroup and a treatment subgroup. A “fixed dose” of a deuterated aminosterol or a salt or derivative thereof for each of the patients in the treatment subgroup is determined using the method described in Example 8. Treatment and wash-out periods mirror Example 8. Patients are monitored for changes in the severity or occurrence of the symptoms. Patients with an underlying disorder are also monitored for changes in other symptoms associated with the disorder. Patients with no underlying disorder are monitored for the development of a constipation associated disorder.

Patients having more severe constipation, e.g., less than 1 spontaneous bowel movement per week, are started at a dose of 75 mg or more. Patients having less severe constipation, e.g., 1 or more SBM/week, are started at a lower dose of aminosterol, e.g., a starting dose of less than 75 mg, for example a dose of 25 mg/day. Thus, the starting deuterated aminosterol dose is dependent upon constipation severity. The full deuterated aminosterol oral dosing range is from about 1 to about 500 mg. Once a prokinetic dose has been identified for a patient, the subject is started at that same dose following drug cessation and reintroduction of drug dosing; e.g., there is no need to ramp up dosing once a prokinetic dose for a patient has been identified.

### Example 10—Hallucinations

This prophetic example describes an exemplary method of (i) treating hallucinations and/or (ii) treating and/or preventing a disorder in which hallucinations are a known symptom (a hallucination-associated disorder) in a subject. The method comprises administering a deuterated aminosterol to a subject in need.

Patients are selected based on having hallucinations. Patients are grouped based on having a particular hallucination-associated disorder or having hallucinations with no underlying disorder. The groups are then subdivided into a control subgroup and a treatment subgroup. A “fixed dose” of a deuterated aminosterol or a salt or derivative thereof for each of the patients in the treatment subgroup is determined using the method described in Example 8, using the improvement of hallucination symptoms as an endpoint. Treatment and wash-out periods mirror Example 8. Patients are monitored for changes in the severity or occurrence of the symptoms. Patients with an underlying disorder are also monitored for changes in other symptoms associated with the disorder. Patients with no underlying disorder are monitored for the development of a hallucination associated disorder.

### Example 11—REM Disturbed Sleep Disorder

This prophetic example describes an exemplary method of (i) treating REM disturbed sleep and/or (ii) treating and/or preventing a disorder in which REM disturbed sleep is a known symptom (a REM disturbed sleep associated disorder) in a subject having REM disturbed sleep. The method comprises administering a deuterated aminosterol to a subject in need.

Patients are selected based on having REM disturbed sleep. Patients are grouped based on having a particular REM disturbed sleep associated disorder or having REM disturbed sleep with no underlying disorder. The groups are then subdivided into a control subgroup and a treatment subgroup. A “fixed dose” of a deuterated aminosterol or a salt or derivative thereof for each of the patients in the treatment subgroup is determined using the method described in Example 8, using the improvement of REM disturbed sleep symptoms as an endpoint. Treatment and wash-out periods mirror Example 8. Patients are monitored for changes in the severity or occurrence of the symptoms. Patients with an underlying disorder are also monitored for changes in other symptoms associated with the disorder. Patients with no underlying disorder are monitored for the development of a REM disturbed sleep associated disorder.

### Example 12—Circadian Rhythm Dysfunction

This prophetic example describes an exemplary method of (i) treating circadian rhythm dysfunction and/or (ii) treating and/or preventing a disorder in which circadian rhythm dysfunction is a known symptom (a circadian rhythm dysfunction associated disorder) in a subject having circadian rhythm dysfunction. The method comprises administering a deuterated aminosterol to a subject in need.

Patients are selected based on having circadian rhythm dysfunction. Patients are grouped based on having a particular circadian rhythm dysfunction associated disorder or having circadian rhythm dysfunction with no underlying disorder. The groups are then subdivided into a control subgroup and a treatment subgroup. A “fixed dose” of a deuterated aminosterol or a salt or derivative thereof for each of the patients in the treatment subgroup is determined using the method described in Example 8, using either the improvement of circadian rhythm dysfunction symptoms as an endpoint. Treatment and wash-out periods mirror Example 8. Patients are monitored for changes in the severity or occurrence of the symptoms. Patients with an underlying disorder are also monitored for changes in other symptoms associated with the disorder. Patients with no underlying disorder are monitored for the development of a circadian rhythm dysfunction associated disorder.

### Example 13—Alzheimer's Disease

This prophetic example describes an exemplary method of treating and/or preventing Alzheimer's disease in a subject in need thereof. The method comprises administering a deuterated aminosterol to a subject in need.

Patients are selected based on being diagnosed with Alzheimer's disease, i.e., having AD, or exhibiting known risk factors of AD, i.e., at risk for developing AD. Patients are grouped based on having AD or at risk for developing AD. The groups are then subdivided into a control subgroup and a treatment subgroup. A “fixed dose” of a deuterated aminosterol or a salt or derivative thereof for each of the patients in the treatment subgroup is determined using the method described in Example 8, using either the improvement of constipation or another symptom of Alzheimer's disease as an endpoint. Treatment and wash-out periods mirror Example 8. Patients are monitored for changes in the severity or occurrence of the symptoms. Patients having AD are monitored for changes in other symptoms associated with the disorder. Patients at risk for developing AD are monitored for the development of AD.

### Example 14—Multiple System Atrophy

This prophetic example describes an exemplary method of treating and/or preventing multiple system atrophy (MSA) in a subject in need thereof. The method comprises administering a deuterated aminosterol to a subject in need.

Patients are selected based on being diagnosed with MSA, i.e., having MSA, or exhibiting known risk factors for MSA, i.e., at risk for developing MSA. Patients are grouped based on having MSA or at risk for developing MSA. The groups are then subdivided into a control subgroup and a treatment subgroup. A “fixed dose” of a deuterated aminosterol or a salt or derivative thereof for each of the patients in the treatment subgroup is determined using the method described in Example 8, using either the improvement of constipation or another symptom of MSA as an endpoint. Treatment and wash-out periods mirror Example 8. Patients are monitored for changes in the severity or occurrence of the symptoms. Patients having MSA are monitored for changes in other symptoms associated with the disorder. Patients at risk for developing MSA are monitored for the development of MSA.

### Example 15—Cognitive Impairment

This prophetic example describes an exemplary method of (i) treating cognitive impairment and/or (ii) treating and/or preventing a disorder in which cognitive impairment is a known symptom (a cognitive impairment related disorder) in a subject having cognitive impairment. The method comprises administering a deuterated aminosterol to a subject in need.

Patients are selected based on having cognitive impairment. Patients are grouped based on having a particular cognitive impairment associated disorder or having cognitive impairment with no underlying disorder. The groups are then subdivided into a control subgroup and a treatment subgroup. A “fixed dose” of a deuterated aminosterol or a salt or derivative thereof for each of the patients in the treatment subgroup is determined using the method described in Example 8, using either the improvement of constipation or cognitive impairment symptoms as an endpoint. Treatment and wash-out periods mirror Example 8. Patients are monitored for changes in the severity or occurrence of the symptoms. Patients with an underlying disorder are also monitored for changes in other symptoms associated with the disorder. Patients with no underlying disorder are monitored for the development of a cognitive impairment associated disorder.

### Example 16—Schizophrenia

This prophetic example describes an exemplary method of treating and/or preventing schizophrenia in a subject in need thereof. The method comprises administering a deuterated aminosterol to a subject in need.

Patients are selected based on being diagnosed with schizophrenia, i.e., having schizophrenia, or exhibiting known risk factors for schizophrenia, i.e., at risk for developing schizophrenia. Patients are grouped based on having schizophrenia or at risk for developing schizophrenia. The groups are then subdivided into a control subgroup and a treatment subgroup. A “fixed dose” of a deuterated aminosterol or a salt or derivative thereof for each of the patients in the treatment subgroup is determined using the method described in Example 8, using either the improvement of constipation or another symptom of schizophrenia as an endpoint. Treatment and wash-out periods mirror Example 8. Patients are monitored for changes in the severity or occurrence of the symptoms. Patients having schizophrenia are monitored for changes in other symptoms associated with the disorder. Patients at risk for developing schizophrenia are monitored for the development of schizophrenia.

### Example 17—Autism

This prophetic example describes an exemplary method of treating and/or preventing autism in a subject in need thereof. The method comprises administering a deuterated aminosterol to a subject in need.

Patients are selected based on being diagnosed with autism. Patients are then divided into a control group and a treatment group. A “fixed dose” of a deuterated aminosterol or a salt or derivative thereof for each of the patients in the treatment group is determined using the method described in Example 8, using either the improvement of constipation or another symptom of autism as an endpoint. Treatment and wash-out periods mirror Example 8. Patients are monitored for changes in the severity or occurrence of the symptoms. Patients having autism are monitored for changes in other symptoms associated with the disorder.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Alexander et al., “Membrane surface charge dictates the structure and function of the epithelial na+/h+ exchanger,” EMBO J., 30:679-691 (2011).
Bhargava et al., “A phase I and pharmacokinetic study of squalamine, a novel antiangiogenic agent, in patients with advanced cancers,” Clin. Cancer Res., 7:3912-9 (2001).
CATE: California Assistive Technology Exchange,” California Assistive Technology Exchange, http://cate.ca.gov/index.cfm?a=Resources&p=News&article=176, Retrieved 2009-Mar.-31.
Connolly et al., “Squalamine lactate for exudative age-related macular degeneration,” Ophthalmol. Clin. North Am., 19:381-91 (2006).
“Cyber Warning Letter”, Center for Drug Evaluation and Research (2002-May-6), http://www.fda.gov/CDER/warn/cyber/2002/CFSANnuGen.htm; Retrieved 2009-Mar.-31.
Genaidy et al., “Effect of squalamine on iris neovascularization in monkeys.” Retina, 22(6): 772-8 (2002).
Genesis, A., “Squalamine trial for the treatment of fibrodysplasia ossificans progressiva initiated”, Angiogenesis Weekly, 8:45 (2002).
Hao et al., “A Phase I and pharmacokinetic study of squalamine, an aminosterol angiogenesis inhibitor,” Clin. Cancer Res., 9(7): 2465-71 (2003).
Herbst et al., “A Phase I/IIA Trial of Continuous Five-Day Infusion of Squalamine Lactate (MSI-1256F) Plus Carboplatin and Paclitaxel in Patients with Advanced Non-Small Cell Lung Cancer 1,” Clinical Cancer Research, 9:4108-4115 (2003).
Higgins et al., “Squalamine improves retinal neovascularization,” Invest. Ophthalmol. Vis. Sci., 41(6): 1507-12 (2000).
Higgins et al., “Regression of retinopathy by squalamine in a mouse model,” Pediatr. Res., 56(1): 144-9 (2004).
Moore et al., “Squalamine: an aminosterol antibiotic from the shark,” Proc. Natl. Acad. Sci. USA, 90(4): 1354-8 (1993).
Rao et al., “Aminosterols from the dogfish shark Squalus acanthias,” J. Nat. Prod., 63(5): 631-5 (2000).
Salmi et al., “Squalamine: an appropriate strategy against the emergence of multidrug resistant gram-negative bacteria?” PLoS ONE, 3(7): e2765 (2008).
Schiller, J. H. and G. Bittner, “Potentiation of platinum antitumor effects in human lung tumor xenografts by the angiogenesis inhibitor squalamine: effects on tumor neovascularization,” Clin. Cancer Res., 5(12): 4287-94 (1999).
Selinsky et al., “The aminosterol antibiotic squalamine permeabilizes large unilamellar phospholipid vesicles,” Biochim. Biophys. Acta., 1370(2): 218-34 (1998).
Selinsky et al., “Squalamine is not a proton ionophore,” Biochim. Biophys. Acta., 1464(1): 135-41 (2000).
Sills et al., “Squalamine inhibits angiogenesis and solid tumor growth in vivo and perturbs embryonic vasculature,” Cancer Res., 58(13): 2784-92 (1998).
Sokoloff et al., “Adjunctive therapy for men with high risk localized and locally advanced prostate cancer: targeting disseminated tumor cells,” J. Urol., 172(6 Pt 2): 2539-44 (2004).
Sumioka et al., “TARP phosphorylation regulates synaptic AMPA receptors through lipid bilayers,” Neuron, 66(5): 755-67 (2009).
Williams et al., “Squalamine treatment of human tumors in nu/nu mice enhances platinum-based chemotherapies,” Clin. Cancer Res., 7(3): 724-33 (2001).;
Yeung et al., “Membrane phosphatidylserine regulates surface charge and protein localization,” Science, 319(5860): 210-3 (2008).
Yin et al., “Antiangiogenic treatment delays chondrocyte maturation and bone formation during limb skeletogenesis,” J. Bone Miner. Res., 17(1): 56-65 (2002).
Yun et al., “Identification of Squalamine in the Plasma Membrane of White Blood Cells in the Sea Lamprey,” Petromyzon marinus,” J. Lipid Res., 48(12): 2579-2586 (2007).
US 2007/10504A1.
U.S. Pat. No. 8,729,058.
U.S. Pat. No. 5,192,756.
Zasloff et al., Int. J. Obes. Relat. Metab. Disord., 25(5):689-97 (2001).
Zasloff, M., “Antimicrobial peptides of multicellular organisms,” Nature, 415(6870): 389-95 (2002).
Zasloff et al., “Squalamine as a broad-spectrum systemic antiviral agent with therapeutic potential,” Proc. Natl. Acad. Sci. USA, 108(38): 15978-83 (2011).

What is claimed is:

1. A compound selected from:
(a) a deuterated aminosterol compound having the structure of 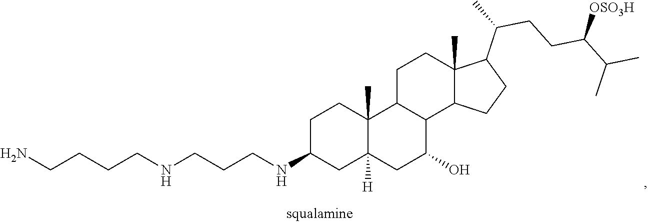
or a pharmaceutically acceptable salt thereof,
wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium; or
(b) a deuterated aminosterol compound having the structure of 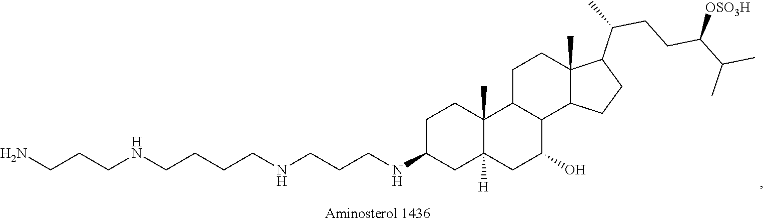
or a pharmaceutically acceptable salt thereof,
wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium; or
(c) a deuterated aminosterol compound having a structure selected from the group consisting of 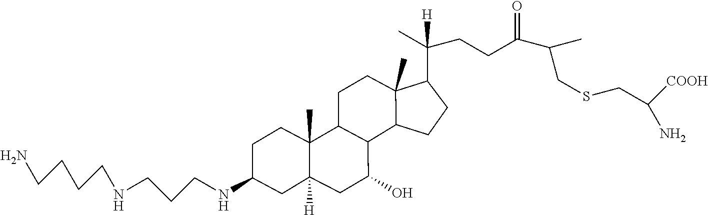 [SMILES: CC(CSCC(N)C(=O)O)C(=O)CC[C@@H](C)C1CCC2C3C(CC[C@@]21C)[C@@]1(C)CC[C@H](NCCCNCCCCN)C[C@@H]1C[C@H]3O]
Compound 1, or a pharmaceutically acceptable salt thereof; 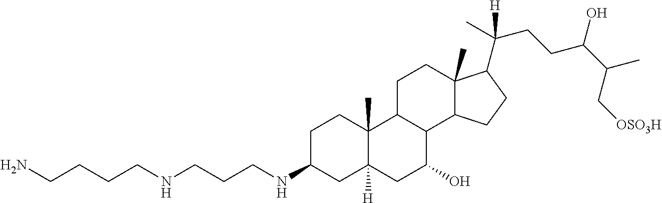
Compound 2, or a pharmaceutically acceptable salt thereof; 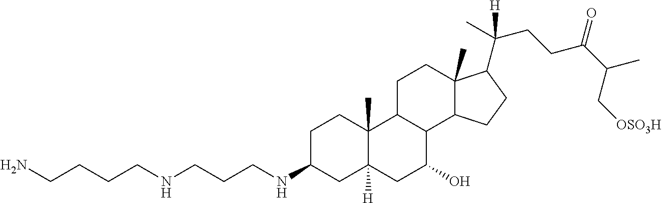
Compound 3, or a pharmaceutically acceptable salt thereof; 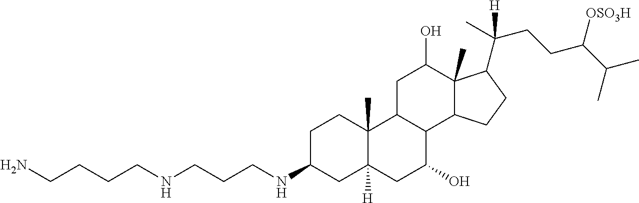
Compound 4, or a pharmaceutically acceptable salt thereof; 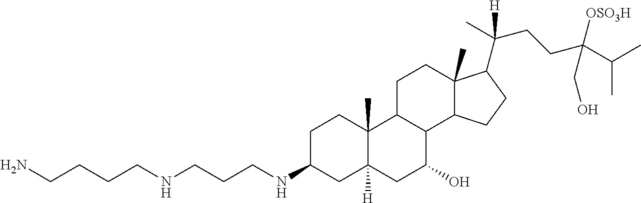
Compound 5, or a pharmaceutically acceptable salt thereof; and 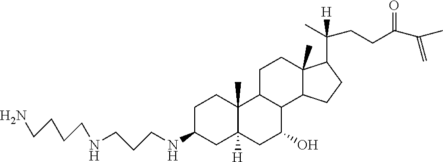 [SMILES: C=C(C)C(=O)CC[C@@H](C)C1CCC2C3C(CC[C@@]21C)[C@@]1(C)CC[C@H](NCCCNCCCCN)C[C@@H]1C[C@H]3O]
Compound 6, or a pharmaceutically acceptable salt thereof,
wherein one or more hydrogen atoms at one or more positions selected from C1, C2, C3, C4, C5, C6, C7, C8, C9, C11, C12, C14, C15, C16, C17, C18, C19, C20, C21, C22, C23, C24, C25, C26, and C27, are replaced with deuterium; or
(d) a compound selected from the group consisting of:

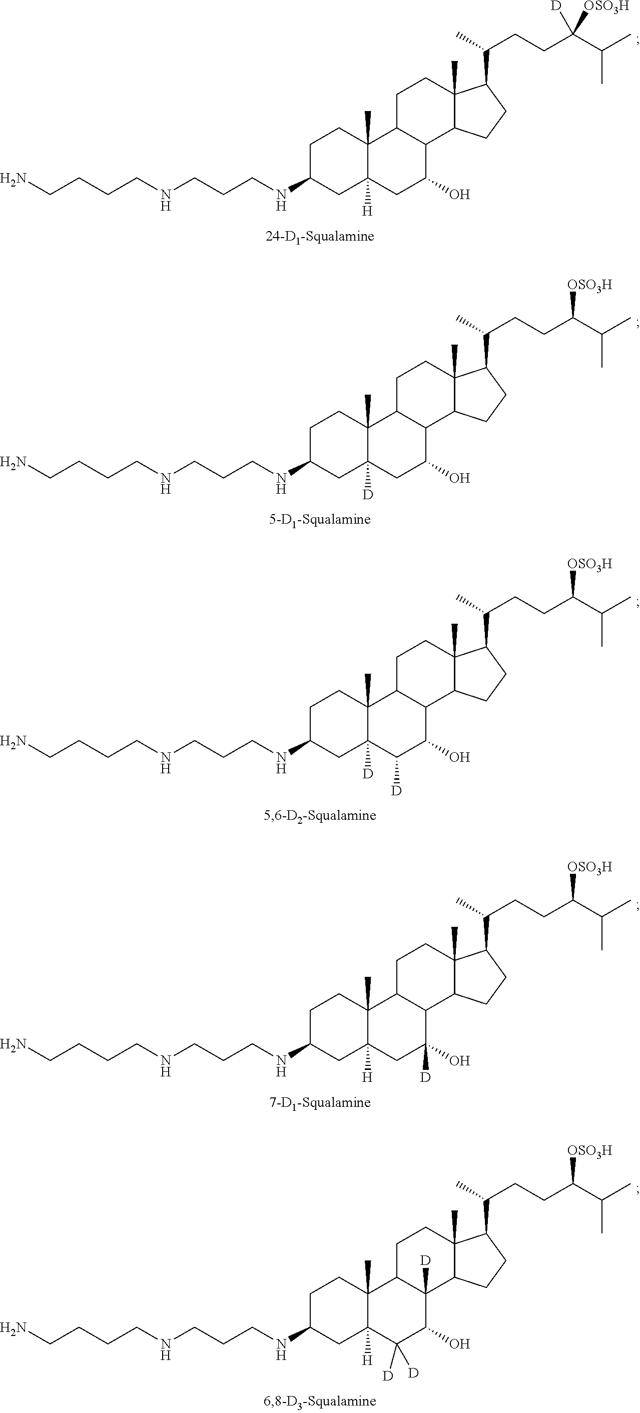

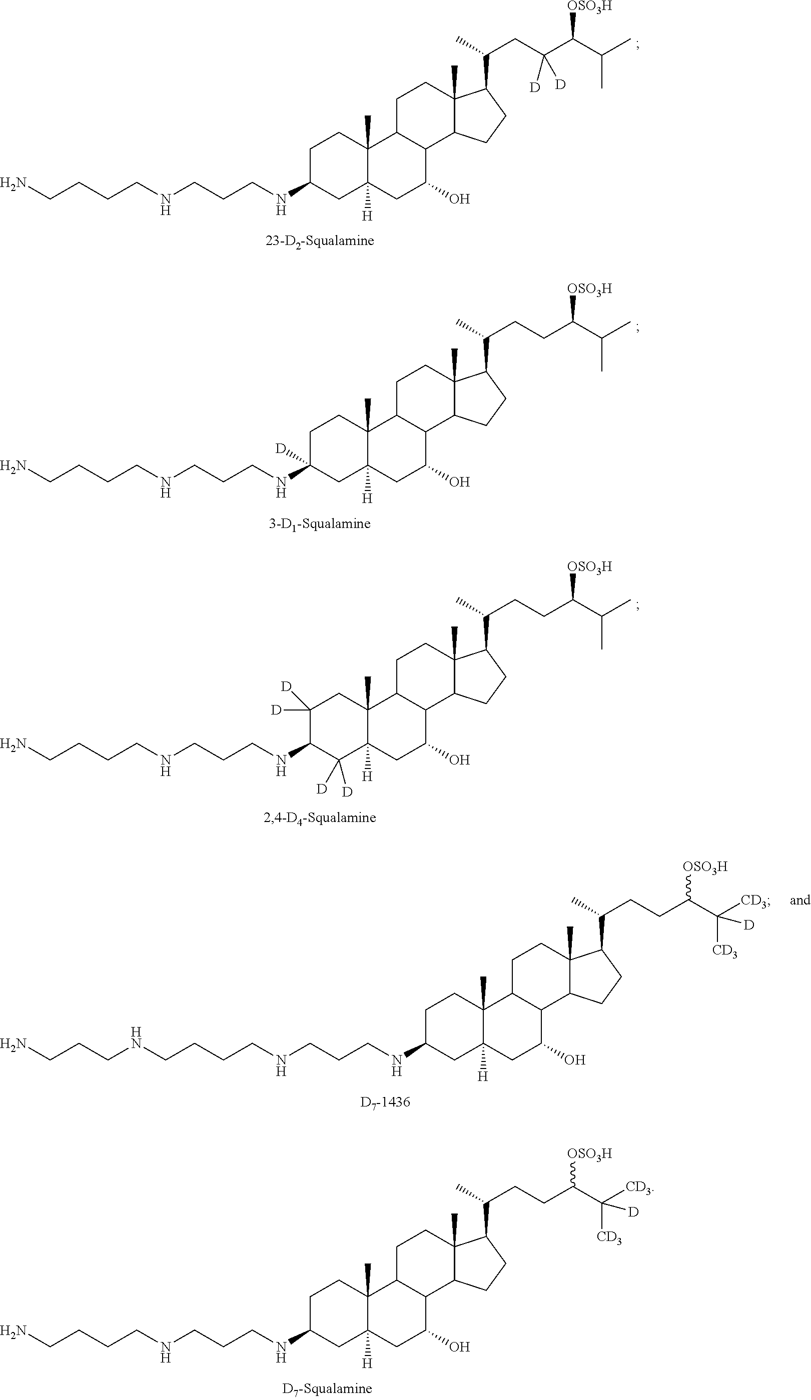

2. The compound of claim 1, or a pharmaceutically acceptable salt thereof, wherein:
(a) one or more hydrogen atoms at one or more positions selected from C2, C3, C4, C5, C6, C7, C8, C22, C23, C24, C25, C26, and C27, are replaced with deuterium; and/or
(b) one or more hydrogen atoms at one or more positions selected from C3, C4, C5, C6, C7, C22, C23, C24, C25, C26, and C27, are replaced with deuterium; and/or
(c) one or more hydrogen atoms at one or more positions selected from C4, C5, C7, C22, C23, C24, C25, C26, and C27, are replaced with deuterium; and/or
(d) one or more hydrogen atoms at one or more positions selected from C1, C2, C3, C4, C5, C6, C7, C8, C11, C12, C16, C17, C18, C19, C20, and C21 are replaced with deuterium; and/or
(e) all hydrogen atoms at positions C25, C26, and C27 are replaced with deuterium; and/or
(f) all hydrogen atoms at positions C26 and C27 are replaced with deuterium.

3. The compound of claim 1, wherein:
(a) any atom not designated as deuterium is present at its natural isotopic abundance; and/or
(b) the deuterium incorporation at each designated deuterium atom is at least about 90%; and/or
(c) the deuterium incorporation at each designated deuterium atom is at least about 95%; and/or
(d) the deuterium incorporation at each designated deuterium atom is at least about 97%.

4. The compound of claim 1, wherein:
(a) the compound has an isotopic enrichment factor selected from the group consisting of at least about 3500, at least about 4000, at least about 4500, at least about 5000, at least about 5500, at least about 6000, at least about 6333.3, at least about 6466.7, at least about 6600, and at least about 6633.3; and/or
(b) the compound has an isotopic enrichment factor of at least 3500 for one deuterium at a single position of the compound; and/or
(c) the compound has a longer half-life as compared to an undeuterated form of the same aminosterol; and/or
(d) the compound has a longer half-life as compared to an undeuterated form of the same aminosterol and wherein the half-life is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

5. The compound of claim 1, which is a phosphate salt.

6. A pharmaceutical composition comprising a compound according to claim 1 and at least one pharmaceutically acceptable excipient or carrier, and optionally wherein the pharmaceutical composition further comprises one or more of the following:
(a) an aqueous carrier;
(b) a buffer;
(c) a sugar; and/or
(d) a polyol compound.

7. A method of treating a subject in need having a condition, disease, or symptom susceptible to treatment with an aminosterol, comprising administering to the subject a therapeutically effective amount of the pharmaceutical composition of claim 6.

8. The method of claim 7, wherein the deuterated aminosterol compound:
(a) has an improved safety profile, as measured by a decrease in incidence of one or more adverse events evaluated using a clinically recognized scale or tool, as compared to the same aminosterol compound which has not been deuterated, and optionally wherein the safety profile is improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(b) has an improved efficacy as compared to the same aminosterol compound which has not been deuterated, and optionally wherein the improved efficacy is measured by improvement of one or more disease symptoms evaluated using a clinically recognized scale or tool, and optionally wherein the efficacy improvement is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(c) has an improved tolerability, measured using a clinically recognized scale or tool, as compared to the same compound which has not been deuterated, and optionally wherein the tolerability is improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) has a reduced required dosage amount and/or dosing frequency, as compared to the same aminosterol compound which has not been deuterated, to obtain the same or improved desired therapeutic effect as measured using a clinically recognized scale or tool, and optionally wherein the dosage is about 10%, about 15%, about 20%, about 25%, about 30%, about 35%, about 40%, about 45%, or about 50% less than that of the same but undeuterated aminosterol.

9. The method of claim 7, wherein:
(a) the composition is administered orally, intranasally, or a combination thereof; and/or
(b) the composition is administered orally and the dose of the compound or a salt or derivative thereof for the subject is from about 25 mg up to about 500 mg/day; and/or
(c) the composition is administered intranasally and the dose of the compound or a salt or derivative thereof for the subject is from about 0.001 mg up to about 6 mg/day.

10. The method of claim 7, wherein:
(a) the method results in improvement or resolution of the condition, disease, or symptom as measured using a clinically recognized scale or tool, and optionally wherein the improvement in the condition, disease, or symptom is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%; and/or
(b) the condition, disease, or symptom to be treated is a gastrointestinal disorder selected from the group consisting of constipation, inflammatory bowel disease, irritable bowel syndrome, and opioid induced constipation; and/or
(c) the condition, disease, or symptom is constipation, and the therapeutically effective amount of the composition is defined as the amount that results in a complete spontaneous bowel movement (CSBM) within 24 hours of dosing on at least 2 of 3 days at a given dose; and/or
(d) the condition to be treated is neurodegeneration or a neurological or neurodegenerative disorder, and optionally wherein the neurodegeneration or neurological or neurodegenerative disorder is (i) age-related; (ii) correlated with age-related dementia; (iii) correlated with a neurodisease; and/or (iv) correlated with one or more conditions or diseases selected from the group consisting of Alzheimer's disease, Parkinson's disease, Lewy Body dementia, fronto temperal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian, spinocerebellar ataxia, hallucinations, depression, autism, and vascular dementia; and/or
(e) the condition to be treated is neurodegeneration or a neurological or neurodegenerative disorder, and optionally wherein (i) progression or onset of the neurodegeneration or neurological or neurodegenerative disorder is slowed, halted, or reversed over a defined time period following administration of the composition, as measured by a medically-recognized technique; and/or (ii) the neurodegeneration or neurological or neurodegenerative disorder is positively impacted by administration of the composition, as measured by a medically-recognized technique; and/or
(f) the condition to be treated is neurodegeneration or a neurological or neurodegenerative disorder, and optionally wherein (i) the positive impact and/or progression of neurodegeneration or neurological or neurodegenerative disorder is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (ii) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(g) the condition, disease, or symptom to be treated is a sleep disorder or sleep disturbance, and optionally wherein administration of the composition decreases the occurrence of at least one symptom of the sleep disorder or disturbance, and optionally wherein the sleep disorder or sleep disturbance comprises a delay in sleep onset, sleep fragmentation, REM-behavior disorder, reduced REM sleep, REM disturbed sleep, sleep-disordered breathing including snoring and apnea, day-time sleepiness, micro-sleep episodes, narcolepsy, reduced total sleep time, sleep problems or sleep disturbances, day-time sleepiness, circadian rhythm disruption or dysfunction, or any combination thereof, and optionally wherein the REM-behavior disorder comprises vivid dreams, nightmares, and acting out the dreams by speaking or screaming, or fidgeting or thrashing of arms or legs during sleep; and/or
(h) the condition, disease, or symptom to be treated is a sleep disorder or sleep disturbance, and optionally wherein the sleep disorder comprises a loss of diurnal rhythm (Circadian rhythm), and optionally wherein the loss of diurnal rhythm is caused by: (i) dysfunction of the suprachiasmatic nucleus; (ii) dysfunction of the enteric nervous system; (iii) dysfunction of the olfactory nervous system; (iv) dysfunction of circadian rhythm caused by visual loss; (v) dysfunction of circadian rhythm caused by jet lag; and/or (vi) dysfunction of circadian rhythm caused by night-shift work; and/or
(i) administration of the composition reverses the dysfunction of the:
(i) suprachiasmatic nucleus, restores the diurnal rhythm, and treats the sleep disorder; and/or
(ii) enteric nervous system, restores the diurnal rhythm, and treats the sleep disorder;
(iii) olfactory system, restores the diurnal rhythm, and treats the sleep disorder;
(iv) circadian rhythm caused by visual loss;
(v) circadian rhythm caused by jet lag; and/or
(vi) circadian rhythm caused by night-shift work.

11. The method of claim 7, wherein:
(a) the sleep disorder is associated with a neurodegenerative disorder, and optionally wherein (i) treating the sleep disorder prevents or delays the onset or progression of the neurodegenerative disorder; and/or (ii) the neurodegenerative disorder is selected from the group consisting of Alzheimer's disease, Parkinson's disease, Lewy Body dementia, fronto temperal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian, spinocerebellar ataxia, hallucinations, depression, autism, and vascular dementia; and/or
(b) the method results in a positive change in the sleeping pattern of the subject, and optionally wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (iii) as a result of the method the subject obtains the total number of hours of sleep recommended by a medical authority for the age group of the subject; and/or
(c) the subject suffers from, is or at risk of developing, hallucinations, and optionally wherein the hallucination comprises a visual, auditory, tactile, gustatory or olfactory hallucination; and/or
(d) the subject suffers from, is or at risk of developing, hallucinations, and optionally wherein the method results in a decreased number or severity of hallucinations of the subject; and/or the method results in the subject being hallucination-free; and/or
(e) the subject suffers from, is or at risk of developing, hallucinations, and wherein the method results in a decreased number or severity of hallucinations of the subject, wherein the decrease in number or severity in hallucinations is defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(f) the subject suffers from, is or at risk of developing, hallucinations, and wherein the hallucination is the result of (i) a neurodegenerative disorder; (ii) a psychiatric disorder; (iii) a neurological disorder; (iv) a brain tumor; (v) a sensory loss; and/or (vi) dysfunction of the enteric nervous system; and/or
(g) the subject suffers from, is or at risk of developing, hallucinations, wherein the hallucination is the result of a neurodegenerative or neurological disorder, and wherein the neurodegenerative or neurological disorder is selected from the group consisting of Alzheimer's disease, Parkinson's disease, Lewy Body dementia, fronto temperal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, autonomic system instability, Guadeloupian, spinocerebellar ataxia, hallucinations, depression, autism, and vascular dementia; and/or
(h) the subject suffers from, is or at risk of developing, hallucinations, wherein the hallucination is the result of a psychiatric disorder, and wherein the psychiatric disorder is selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia; and/or
(i) the subject suffers from, is or at risk of developing, hallucinations, wherein the hallucination is the result of a neurodegenerative or neurological disorder, and wherein the neurodegenerative or neurological disorder is the result of:
(i) a sleep disorder;
(ii) a focal brain lesion;
(iii) a focal brain lesion which is occipital lobe lesions or temporal lobe lesions;
(iv) a temporal lobe lesion selected from the group consisting of lesions of the uncinate gyrus, cerebral peduncles, and substantia nigra;
(v) a diffuse involvement of the cerebral cortex;
(vi) a diffuse involvement of the cerebral cortex caused by a viral infectious disease;
(vii) a diffuse involvement of the cerebral cortex caused by a viral infectious disease, wherein the viral infectious disease is selected from the group consisting of acute metabolic encephalopathies, encephalitis, and meningitis;
(viii) a diffuse involvement of the cerebral cortex caused by a cerebral vasculitis condition;
(ix) a diffuse involvement of the cerebral cortex caused by a cerebral vasculitis condition, wherein the cerebral vasculitis condition is caused by an autoimmune disorder, a bacterial or viral infection, or a systemic vasculitis; and/or
(x) a diffuse involvement of the cerebral cortex caused by a cerebral vasculitis condition, wherein the cerebral vasculitis condition is caused by an autoimmune disorder which is Systemic Lupus Erythematosus (SLE); and/or
(j) the subject suffers from, is or at risk of developing, hallucinations, and wherein the hallucination is the result of a sensory loss, and wherein the sensory loss is visual, auditory, gustatory, tactile, or olfactory;
(k) administration of the composition:
(i) reverses dysfunction caused by the neurodegenerative or neurological disorder and treats and/or prevents the hallucination;
(ii) reverses dysfunction caused by the psychiatric disorder and treats and/or prevents the hallucination;
(iii) reverses dysfunction caused by the sensory loss and treats and/or prevents the hallucination; and/or
(iv) reverses dysfunction of the enteric nervous system and treats and/or prevents the hallucination; and/or
(l) the subject suffers from, is or at risk of developing, depression, and optionally wherein the method results in improvement in a subject's depression, as measured by one or more clinically-recognized depression rating scale; and/or
(m) the subject suffers from, is or at risk of developing, depression, wherein the method results in improvement in a subject's depression, and wherein the improvement is in one or more depression characteristics selected from the group consisting of mood, behavior, bodily functions such as eating, sleeping, energy, and sexual activity, and/or episodes of sadness or apathy; and/or the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(n) the subject suffers from, is or at risk of developing, autism, and optionally wherein the method results in improvement in one or more of the subject's autism characteristics or behaviors, as measured by a clinically-recognized rating scale; and/or in one or more autism characteristics or behaviors selected from the group consisting of social skills, repetitive behaviors, speech, nonverbal communication, sensory sensitivity, behavior, social interaction, and communication skills, as measured using a clinically-recognized scale; and/or
(o) the subject suffers from, is or at risk of developing, autism, and wherein the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%; and/or
(p) the subject suffers from, is or at risk of developing, schizophrenia, and optionally wherein the method results in improvement in one or more schizophrenia characteristics or behaviors, as measured using a clinically recognized rating scale, and optionally wherein the schizophrenia characteristics or behaviors are selected from the group consisting of unclear or confusing thinking, reduced social engagement, reduced emotional expression, abnormal social behavior, failure to understand reality, lack of motivation, and hearing voices that others do not hear, as measured using a clinically-recognized scale, and optionally wherein the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(q) the subject suffers from, is or at risk of developing, an inflammatory disease or condition caused by excessive expression or concentration of alpha synuclein in the subject, and optionally wherein the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment, and optionally wherein the decrease is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, and optionally wherein the method results in a decrease in concentration of alpha synuclein in the subject, and optionally wherein the decrease in alpha-synuclein concentration in is measured qualitatively, quantitatively, or semi-quantitatively by one or more methods selected from the group consisting of:
(i) first determining the concentration of alpha-synuclein in a tissue sample from the subject prior to treatment, followed by: (A) after treatment determining the alpha-synuclein concentration in the same tissue type from the same subject; or (B) after treatment comparing the alpha-synuclein concentration in the same tissue type to a control;
(ii) measuring the intensity of inflammation over time;
(iii) measuring the amount of inflammatory markers over time;
(iv) measuring the amount of inflammatory markers in blood, plasma, or tissue over time, either qualitatively or quantitatively;
(v) measuring the amount of one or more inflammatory marker cytokines in blood, plasma, or tissue over time, either qualitatively or quantitatively;
(vi) measuring the amount of one or more plasma markers of inflammation such as TNF, IL-8, or CRP in blood, plasma, or tissue over time, either qualitatively or quantitatively; and
(vii) measuring the amount of inflammatory cells in blood, plasma, or tissue over time, either qualitatively or quantitatively; and/or
(r) the subject suffers from, is or at risk of developing, an inflammatory disease or condition caused by excessive expression or concentration of alpha synuclein in the subject and wherein the method is applied to a patient population susceptible to excessive expression of alpha-synuclein, resulting in an excessive or high concentration of alpha-synuclein; and/or
(s) the condition to be treated is an infection selected from the group consisting of viral infections, antimicrobial infections, Gram-negative and Gram-positive bacterial infections, Mycobacteria infections, fungal infections, and protozoan infections; and/or
(t) the condition is a disease state known to be associated with pathological neovascularization, selected from the group consisting of cancer, vascular disorders of the eye, macular degeneration, age-related macular degeneration, retinopathy of prematurity, corneal neovascularization, diabetic retinopathy, fibrodysplasia ossificans progressiva, and disorders of neovascularization; and/or
(u) the condition to be treated is obesity.

12. The method of claim 7, wherein:
(a) the composition is taken on an empty stomach, optionally within two hours of the subject waking; and/or
(b) no food is taken after about 60 to about 90 minutes of taking the composition; and/or
(c) the subject is a human.

13. The method of claim 7, wherein:
(a) the composition is administered in combination with at least one additional active agent to achieve either an additive or synergistic effect, and optionally wherein the additional active agent is administered via a method selected from the group consisting of concomitantly; as an admixture; separately and simultaneously or concurrently; and separately and sequentially; and/or
(b) the composition is administered in combination with at least one additional active agent to achieve either an additive or synergistic effect, and optionally wherein the additional active agent is a different aminosterol from that administered in method of claim 7;
(c) the composition is administered in combination with at least one additional active agent to achieve either an additive or synergistic effect, comprising comprising (i) orally administering a composition comprising a deuterated aminosterol compound according to claim 1; and (ii) intranasally administering a composition comprising a compound according to claim 1.

14. A process for preparing D₇-1436, the process comprising:
(a) reacting compound (A) 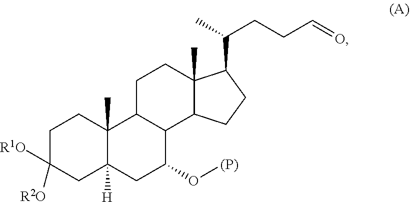
with an organometallic reagent to provide compound (B): 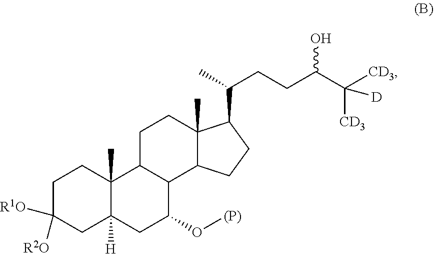
wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether;
(b) reacting compound (B) with sulfur trioxide-pyridine complex to provide compound (C) 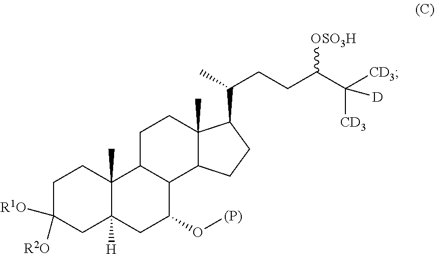
(c) reacting compound (C) under acidic conditions to provide compound (D) 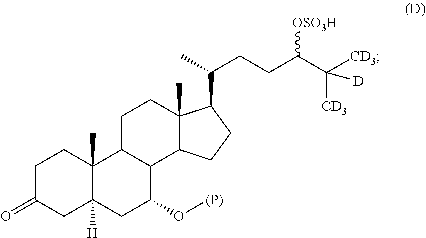
(d) reacting compound (D) under reductive amination conditions to provide compound (E) 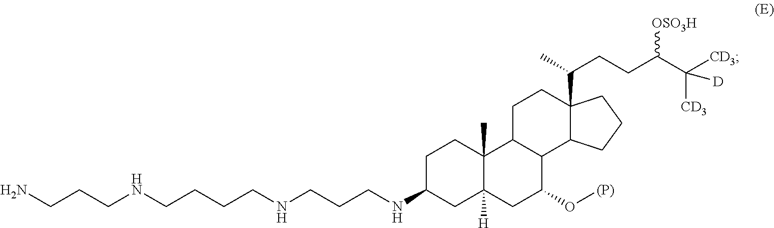
and
(e) reacting compound (E) under conditions to remove P to provide D₇-1436

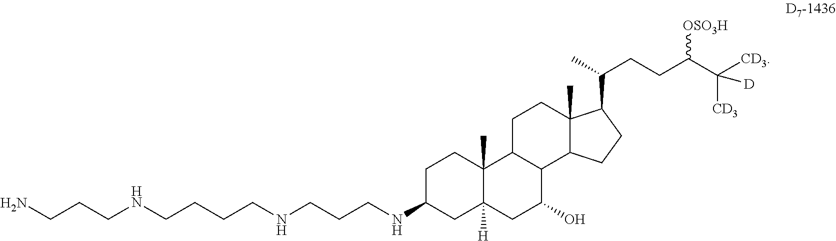

15. A process for preparing D₇-Squalamine, the process comprising:
(a) reacting compound (A) 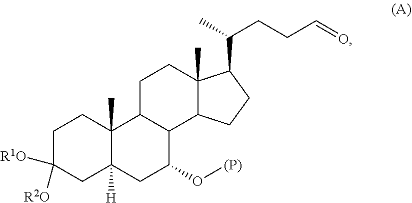
with an organometallic reagent to provide compound (B): 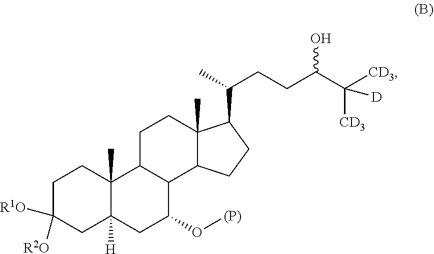
wherein each of R¹ and R² is independently C₁₋₆ alkyl, or R¹ and R², together with the oxygen atoms to which they are attached, form a heterocyclic ring; and P is substituted or unsubstituted C₁₋₆ alkyl ester, substituted or unsubstituted aryl ester, substituted or unsubstituted heteroaryl ester, or substituted or unsubstituted C₁₋₆ alkyl ether;
(b) reacting compound (B) with sulfur trioxide-pyridine complex to provide compound (C) 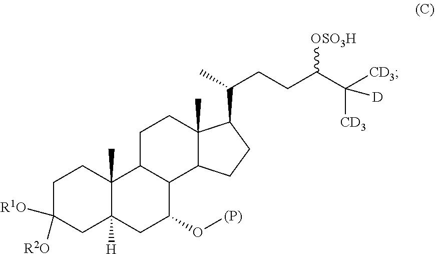
(c) reacting compound (C) under acidic conditions to provide compound (D) 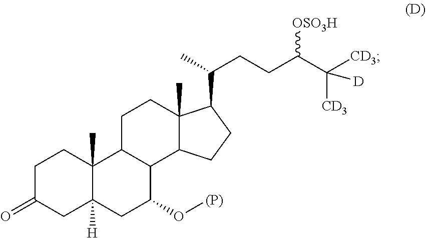
(d) reacting compound (D) under reductive amination conditions to provide compound (F) 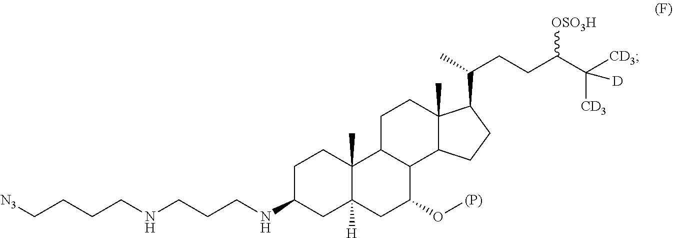
(e) reacting compound (F) under reducing conditions to provide compound (G) 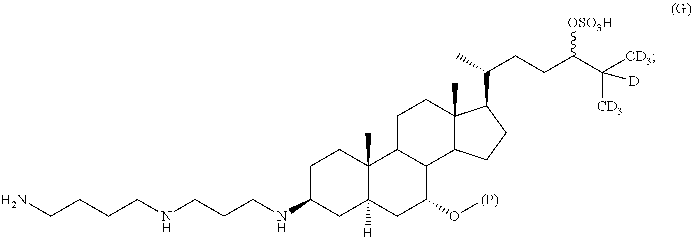
and
(f) reacting compound (G) under conditions to remove P to provide D₇-Squalamine

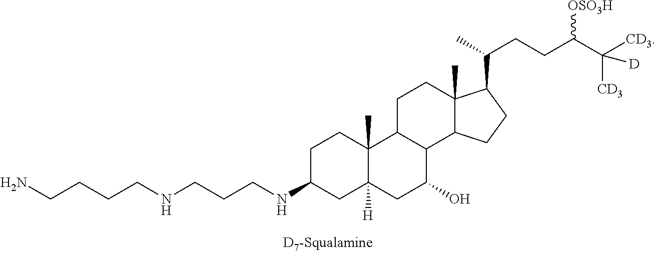

16. A compound selected from the group consisting of:

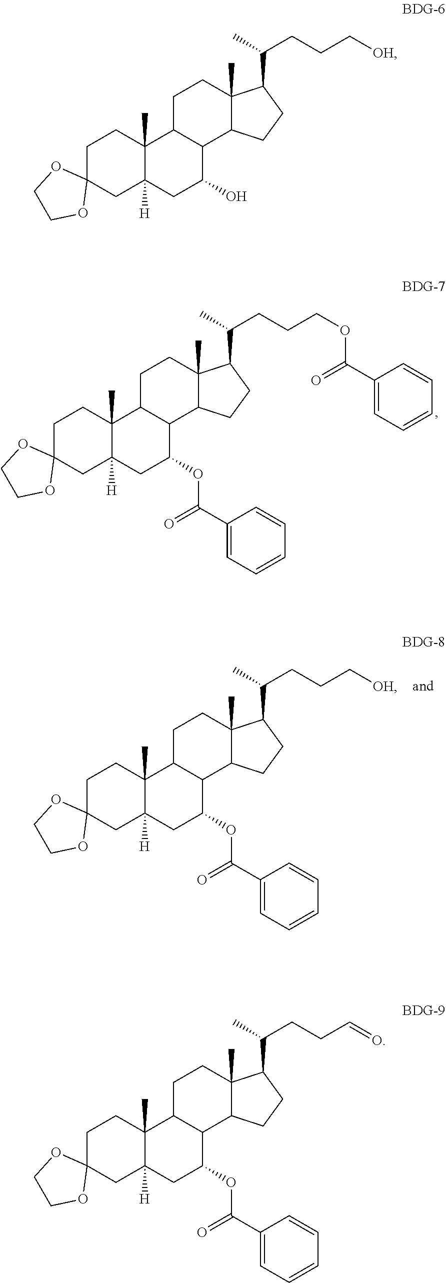 [SMILES: C[C@H](CCCO)[C@H]1CCC2C3C(CC[C@@]21C)[C@@]1(C)CCC2(C[C@@H]1C[C@H]3O)OCCO2 ; C[C@H](CCCOC(=O)c1ccccc1)[C@H]1CCC2C3C(CC[C@@]21C)[C@@]1(C)CCC2(C[C@@H]1C[C@H]3OC(=O)c1ccccc1)OCCO2 ; C[C@H](CCCO)[C@H]1CCC2C3C(CC[C@@]21C)[C@@]1(C)CCC2(C[C@@H]1C[C@H]3OC(=O)c1ccccc1)OCCO2 ; C[C@H](CCC=O)[C@H]1CCC2C3C(CC[C@@]21C)[C@@]1(C)CCC2(C[C@@H]1C[C@H]3OC(=O)c1ccccc1)OCCO2]

17. A process for preparing compound (O), the process comprising:
(a) reacting compound (H) 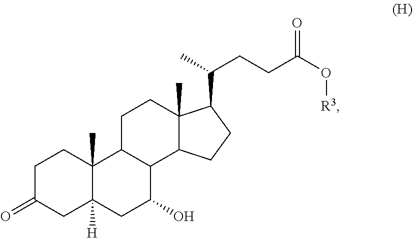
under acidic conditions to provide compound (J) 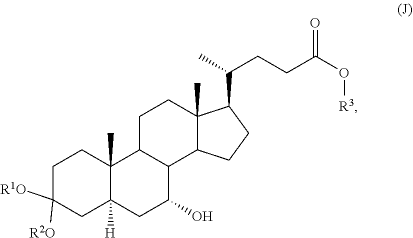
wherein each of R¹ and R² is independently C₁₋₆ alkyl, or R¹ and R², together with the oxygen atoms to which they are attached, form a heterocyclic ring; and R³ is substituted or unsubstituted C₁₋₆ alkyl;
(b) reacting compound (J) with a reducing agent to provide compound (K) 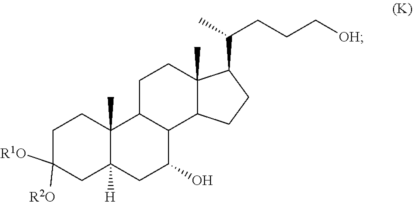
(c) reacting compound (K) under conditions to introduce a protecting group P′ to provide compound (L) 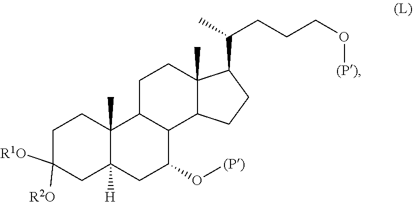
wherein P′ is substituted or unsubstituted C₁₋₆ alkyl ester, substituted or unsubstituted aryl ester, or substituted or unsubstituted heteroaryl ester;
(d) reacting compound (L) under conditions to selectively remove one P′ to provide compound (M) 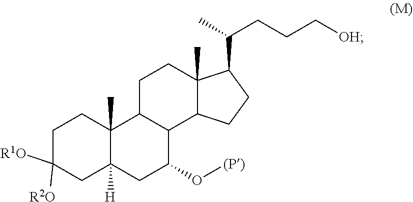
(e) reacting compound (M) under oxidation conditions to provide compound (N) 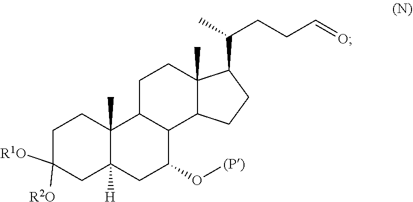
and
(f) reacting compound (N) with an organometallic reagent to provide compound (O)

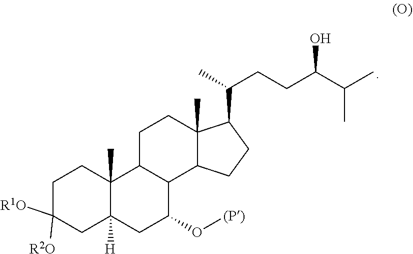

18. A process for preparing compound (O), the process comprising:
(a) reacting compound (P) 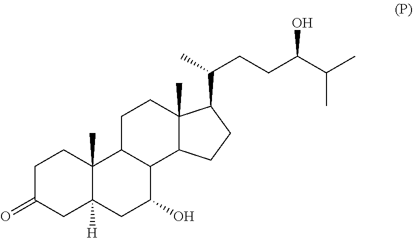 [SMILES: CC(C)[C@H](O)CC[C@@H](C)[C@H]1CCC2C3C(CC[C@@]21C)[C@@]1(C)CCC(=O)C[C@@H]1C[C@H]3O]
under acidic conditions to provide compound (Q): 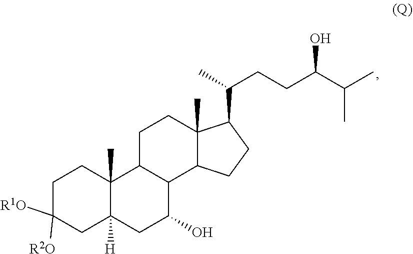
wherein each of R¹ and R² is independently C₁₋₆ alkyl, or R¹ and R², together with the oxygen atoms to which they are attached, form a heterocyclic ring;
(b) reacting compound (Q) under conditions to introduce a protecting group P′ to provide compound (R) 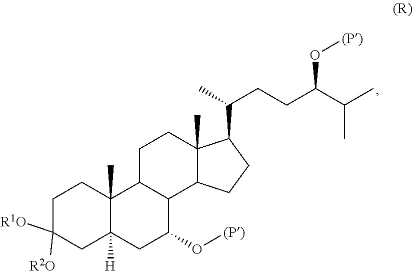
wherein P′ is substituted or unsubstituted C₁₋₆ alkyl ester, substituted or unsubstituted aryl ester, or substituted or unsubstituted heteroaryl ester; and
(c) reacting compound (R) under conditions to selectively remove one P′ to provide compound (O)

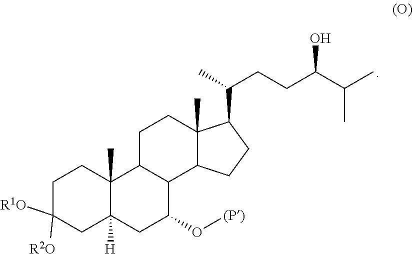

19. A process for preparing CBZ-spermidine: 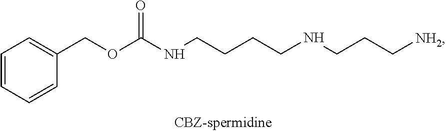 [SMILES: NCCCNCCCCNC(=O)OCc1ccccc1]
or a salt thereof, the process comprising:
(a) reacting 4-amino-1-butanol under conditions to introduce a Cbz group to provide compound (S) 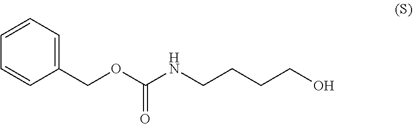 [SMILES: O=C(NCCCCO)OCc1ccccc1]
(b) sulfonylating compound (S) to provide compound (T) 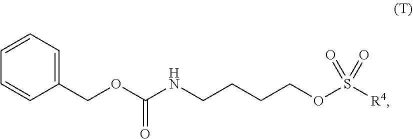
wherein R⁴ is substituted or unsubstituted C₁₋₆ alkyl, or substituted or unsubstituted aryl; and
(c) reacting compound (T) with 1,3-diaminopropane to provide CBZ-spermidine, or the salt thereof.
